(19) United States
(12) Patent Application Publication
(10) Pub. No.: US 2020/0040410 A1
(43) Pub. Date: Feb. 6, 2020

(54) GENOME-SCALE HIGH-RESOLUTION MAPPING OF ACTIVATING AND REPRESSIVE NUCLEOTIDES IN REGULATORY REGIONS

(71) Applicants: THE REGENTS OF THE UNIVERSITY OF CALIFORNIA, Oakland, CA (US); MASSACHUSETTS INSTITUTE OF TECHNOLOGY, Cambridge, MA (US)

(72) Inventors: Jason ERNST, Los Angeles, CA (US); Manolis KELLIS, Cambridge, MA (US)

(73) Assignees: The Regents of the University of California, Oakland, CA (US); Massachusetts Institute of Technology, Cambridge, MA (US)

(21) Appl. No.: 16/337,819

(22) PCT Filed: Sep. 29, 2017
(86) PCT No.: PCT/US2017/054366

§ 371 Date: Mar. 28, 2019

Related U.S. Application Data
(60) Provisional application No. 62/402,930, filed on Sep. 30, 2016.

(51) Int. Cl. C12Q 1/6897; G16B 20/30; G16B 20/20; C12Q 1/6827; G16B 5/20

(52) U.S. Cl. CPC C12Q 1/6897; G16B 20/30; G16B 20/20; C12Q 2600/166; G16B 5/20; C12Q 2537/165; C12Q 1/6827

(57) ABSTRACT

Disclosed is a method of determining regulatory activity of nucleotides in a genome, which includes: (1) designating a reference region of the genome; (2) designating a plurality of reporter tiles covering the reference region, where each reporter tile has a length L, the reporter tiles are offset from one another within the reference region with a step sizes, and a ratio of s to L is 1:5 or less; and (3) generating a plurality of reporter constructs corresponding to the reporter tiles.

### CROSS-REFERENCE TO RELATED APPLICATION

This application claims the benefit of U.S. Provisional Application No. 62/402,930, filed Sep. 30, 2016, the content of which is incorporated herein by reference in its entirety.

### STATEMENT REGARDING FEDERALLY SPONSORED RESEARCH OR DEVELOPMENT

This invention was made with Government support under 1254200, awarded by the National Science Foundation, and GM113708, awarded by the National Institutes of Health. The Government has certain rights in the invention.

### TECHNICAL FIELD

This disclosure generally relates to determining, analyzing, and mapping regulatory activities of nucleotides of a genome.

### BACKGROUND

Epigenome maps predict thousands of putative regulatory regions through their in vivo epigenomic signatures and are widely used for studying gene regulation and disease. However, such maps present indirect evidence of regulatory function, have often constrained resolution and typically do not distinguish activator from repressor nuclei acid elements. DNA motif and sequence pattern analysis can complement epigenome maps, but also provides indirect evidence and identifies sequences that match enriched patterns.

Episomal reporter assays and endogenous modulation are two complementary approaches to characterize putative regulatory regions. Episomal reporters evaluate sequence function directly, independently of epigenetic effects, whereas endogenous perturbations capture endogenous context effects. Multiplexed endogenous or episomal assays can be used to dissect few regulatory regions at high resolution or many at low resolution.

Massively parallel reporter assays (MPRAs) synthesize DNA sequences on programmable microarrays and integrate them in reporter gene plasmids that are then transfected into cell types of interest. Barcodes placed in reporter gene 3′ untranslated regions (UTRs) (to reduce their effect on pre-transcriptional control) provide a quantitative readout of gene expression. The finite number of array spots constrains the number of regions tested and the number of reporter constructs devoted to each region. Owing to the short length of synthesized fragments (e.g., 145 nucleotides), MPRAs involve accurate knowledge of putative regulatory region position and boundaries, which are not generally known.

It is against this background that a need arose to develop the embodiments described herein.

### SUMMARY

MPRAs allow nucleotide-resolution dissection of transcriptional regulatory regions, such as enhancers, but just few regions at a time. Herein disclosed for some embodiments is a combined experimental and computational approach, systematic high-resolution activation and repression profiling with reporter tiling using MPRA (Sharpr-MPRA), that allows high-resolution analysis of a large number of regions. Sharpr-MPRA combines dense tiling of overlapping MPRA constructs with a probabilistic graphical model to recognize functional regulatory nucleotides, and to distinguish activating and repressive nucleotides, using their inferred contribution to reporter gene expression.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1. Experimental design. (a) Comparison of MPRA strategies for testing regulatory regions. Non-tiling approaches (top) use multiple barcodes for the same tested sequence. The pilot design (middle) tests each region using nine tile offsets, spaced at 30-base pair (bp) increments, each tested using 24 barcodes (216 MPRA array spots per region). The scaled-up design (bottom), tests each region using 31 tile offsets spaced at 5-bp increments, each tested using a single barcode per tile offset. The designs are represented to scale along the horizontal dimension. Top and bottom are represented to scale in the vertical dimension. (b) The 25 chromatin states used in selecting regulatory regions for testing in the scale-up design (FIG. 11). Heatmap indicates the emission probabilities (scaled between 0 and 100) for each epigenomic feature (columns) in each chromatin state (rows). Tested regions were selected to DNase sites in one of four cell types, with the number of regions selected based on a stratified random sampling as indicated. (c) Overview of experiments using the scale-up design (see FIG. 7 for the pilot design). 16 experiments are carried out, including two sets of 7,860 regions (row groups) across 25 chromatin states (shading), with 31 tiles per region (individual columns), each tested in both HepG2 and K562 cells, each using both a minimal promoter (minP) and an SV40 promoter (SV40P), each in two replicates (Rep1 and Rep2). Heatmap shows MPRA reporter gene expression measurements (blue=low, yellow=high, black=missing).

FIG. 2. Tiling enhancer regions in pilot design revealed regulatory segments at 30-bp resolution. (a) Effect of tile offset and H3K27ac dip score on reporter expression. Average HepG2 cell reporter expression (y axis) at each of nine offsets (x axis) for three sets of regions: HepG2 cell candidate enhancers with the highest H3K27ac dip scores, candidate enhancers with a range of dip scores, and regions that were not predicted enhancers in HepG2 cells but were predicted enhancers with a high dip score in K562 cells. Error bars, standard error of mean (s.e.m.) (n=100, 100 and 50, respectively). (b) Consecutive tiles can differ in reporter expression. Comparison of median reporter activity between biological replicates in HepG2 cells (top; first eight tile offsets are shown). Comparison of consecutive tiles T (x axis) and T+1 (y axis) for the same biological replicate (rep1) (bottom). (c) Chromatin state annotations in nine cell types and H3K27ac signal track in HepG2 cells over about 400 kb and about 10 kb surrounding the tiled 385-bp region centered in the H3K27ac dip (top). Expanded view of tile reporter measurements across all nine tiles, 24 barcodes, and two replicates (middle). Tiles #4 and #5 share 115 bp in common (abbreviated), and have 30 bp unique to #4 or #5 (shown), indicating the potential presence of activating elements in the sequence unique to #5 and/or repressive elements in the sequence unique to #4 (bottom). Indeed, the 30-bp segment unique to #5 contains a candidate binding site for HNF4, an activator of liver-related functions. (d) Expanded view of expression activity measurements for consecutive tiles #4 and #5 for all individual barcodes (points), sorted by their reporter expression levels. For replicate 1 of tile #4, 1 of 24 barcode measurements failed. The y-axis coordinates correspond to the ones shown in (b). Horizontal lines indicate median normalized MPRA activity. See FIGS. 8-10 for additional results from the pilot design.

FIG. 3. Scale-up design permits dissection of regulatory regions at high resolution. (a) Modeling scheme and probabilistic graphical model for the scale-up design. Variables M₁, . . . , M₃₁ represent the observed values of the reporter measurements for the 31 tiles (each 145 bp long), and variables A₁, . . . , A₅₉ represent the unobserved regulatory activity level of each 5-bp interval of the 295 bp covered, which is then normalized into the Sharpr-MPRA regulatory activity score. Probabilistic graphical model (bottom) used for high-resolution inference of activating and repressive intervals, with arrows Aₖ→Mⱼ illustrating the dependencies between variables when tile Mⱼ overlaps interval Aₖ, and the direction of information flow in the generative model. Conditional inference allows the use of the observed reporter measurements M₁, . . . , M₃₁ for the 31 tiles to infer the unobserved activity levels A₁, . . . , A₅₉ for the 59 intervals of length 5 bp each, which is interpolated to each nucleotide position i, under the modeling assumptions specified in Methods. (b) Observed reporter expression measurements for 145-bp segments (top) and inferred regulatory activity for 5-bp segments, interpolated to individual nucleotides (bottom) for two 295-bp regulatory regions in HepG2 cells. At each offset, the four rows correspond to four measurements of the same tile, using minP and SV40P, each in two replicates (top). Measurements for each tile are shown spanning all nucleotide positions the tile covers. White rows represent missing data for a promoter/replicate combination for a given 145-bp tile. Resulting inference of regulatory activity at each nucleotide i is shown using all four measurements, using the two SV40P measurements, or using the two minP measurements. Predicted positions of highest activating (positive scores) or repressive (negative scores) activity capture CENTIPEDE predicted binding sites and conserved elements identified by the SiPhy-PI method, even though such information was not used in the inferences. (c) Higher activating or repressive Sharpr-MPRA regulation activity score in HepG2 cells (x axis) resulted in higher overlap with transcription factor binding sites predicted by CENTIPEDE in HepG2 cells (y axis, left), and higher overlap with conserved elements identified by SiPhy-PI (y axis, right). Each point represents the average of 927 nucleotide positions in each of 5,000 quantiles. Horizontal dark line shows the expected overlap averaged across all 295 nucleotide positions of each region, and the additional horizontal line shows the expected overlap fraction at the center nucleotide position (a stringent control). Reversed gray barplot at the top of each panel shows the density (histogram) of the distribution of Sharpr-MPRA combinedP scores in HepG2 cells. (d) Sharpr-MPRA inferences capture regulatory nucleotides at high resolution. Cumulative overlap (y axis) with CENTIPEDE predicted transcription factor binding sites in HepG2 cells (left) and evolutionarily conserved elements (right) is higher for MaxPos, than for the stringent control of CenPos or for SymPos, indicating this is not a positional bias. Each set is ranked from highest (left) to lowest (right) absolute Sharpr-MPRA score in MaxPos/CenPos/SymPos nucleotides (x axis) in HepG2 cells (see FIG. 27 for K562 cells, and for individual promoter types). Dotted lines mark thresholds at absolute score ≥2, ≥1 and ≥0.5. MaxPos, CenPos and SymPos nucleotide positions are illustrated in (b).

FIG. 4. Comparison of Sharpr-MPRA with motif annotations. (a) Comparison of average Sharpr-MPRA score for regulatory motifs from an assembled compendium (points) in HepG2 vs. K562 cells, averaged at the center position of all instances for each motif. Arrows highlight motif examples mentioned in the text. Motifs with more than 10 instances are shown. (b) Aggregation plots of the regulation score (y axis) at increasing varying genomic positions relative to the motif center (x axis) for K562 and HepG2 cells for all motif instances, predicted independently of cell types, for ETS, GATA, REST, HNF4, and RFX5 regulatory motifs. Error bar height is one s.e.m. (c) Activating enrichment score and repressive enrichment score for the regulatory motif compendium (points) in HepG2 and K562 cells, based on the statistical significance (−log 10 P) for the enrichment of the center motif position for nucleotides with Sharpr-MPRA scores ≤−1 (repressive) or ≤1 (activating), using a one-sided binomial test. Inset expands boxed region, and does not cover any points, as no motif was enriched beyond −log 10P=20 for both activating and repressing positions. Arrows highlight members of MAF and AP-2 motif families discussed in the text. Similar plots using top 5% activating and repressive nucleotides are shown in FIG. 35.

FIG. 5. Regulatory activity of ERV1 and LINE repeats. (a,b) For nucleotides of varying Sharpr-MPRA regulatory activity score in HepG2 cells, the fraction that overlaps with annotated repeat elements showed strong ERV1 repeat enrichment at the most activating nucleotides (a) and a depletion for LINE repeats at the most activating and most repressive nucleotides (b). Bins were formed by assigning each base to the nearest 0.5 value based on its regulatory score. Extreme bins contain extreme values as indicated. Horizontal lines denote expected overlap based on center position (CenPos) and all positions. Enrichments and depletions for K562 cells and for additional repeats are shown in FIG. 36.

FIG. 6. Endogenous chromatin state is predictive of reporter activity. (a,b) Average HepG2 cell Sharpr-MPRA regulatory score (y axis) and standard error (vertical error bars) for each chromatin state (columns) for all 3,930 DNase sites selected in HepG2 cells (a) and all 15,720 regions selected in all four cell types (b), evaluated at nucleotide positions of maximum absolute activity (MaxPos). In (a), each group of consecutive bars shows the combinedP, minP and SV40P results. All 3,930 regions correspond to DNase sites in HepG2 cells, as the regions were selected in HepG2 cells. In (b), the combinedP score is shown separately for regions corresponding to DNase (light shading) and non-DNase (darker shading) sites in HepG2 cells. Some DNase sites selected in other cell types were also DNase in HepG2, leading to an increased DNase count compared to data in (a). All non-DNase sites in HepG2 cells were DNase sites in the cell type in which they were selected. The chromatin state of the center position is shown. K562 cells plots in FIG. 37. (c) For all motifs (circles) in HepG2 cell-selected regions (top) and K562 cell-selected regions (bottom), relationship between their average combinedP Sharpr-MPRA score in the corresponding cell type (y axis) and their expected score based on the chromatin states in which the motif occurs (x axis), quantified as the median of randomized motif occurrences that preserve positional and chromatin state distributions (Methods). Motifs with 20 or more evaluated instances in selected regions are shown. Randomization 95^{th} percentile confidence intervals are shown in FIG. 45.

FIG. 7. Pilot design (a) Overview of the selection of regions and experiments. Two hundred fifty regulatory regions were selected to be tested with 100 being selected based on being in an HepG2 candidate enhancer state and having a high H3K27ac dip score, 100 being selected based on being in an HepG2 candidate enhancer state and covering a range of H3K27ac dip scores, and 50 being selected based on being in a K562 candidate enhancer state with a high H3K27ac dip score in K562 and in a low-activity state in HepG2 (see Methods). These regulatory regions were tested in both K562 and HepG2 using a SV40 promoter in replicate. (b) Chromatin state model used for selecting strong enhancer regions in HepG2 and K562, and low-activity regions in K5623. The frequency of each chromatin state mark is shown (scaled between 0 and 100). Note that for the scale-up model, a richer 25-state model is used to capture a higher diversity of chromatin states (shown in FIG. 11). (c) The first two larger columns are the H3K27ac signal in HepG2 and K562 cells respectively, while the next two larger columns are the DNase signal in HepG2 and K562 cells respectively. Each larger column is separated by white lines. Within each larger column is a heatmap of the signal across the 385-bp region based on the color scale shown at bottom. The first larger set of rows (separated by horizontal white lines) are those regions corresponding to the HepG2 tiled enhancer regions with a high dip score, the second are those regions corresponding to the HepG2 tiled enhancer regions with a range of dip scores, and the third set are the K562 based tiled enhancer regions. Within each set, the regions are ordered in terms of decreasing dip score. (d) Heatmap of the reporter expression values. The first two larger columns show the results for the experiments in two HepG2 replicates and the next two columns show the experiments in the two K562 replicates. Within each larger column is the median log-base two ratio reporter expression values at each of the nine tile offsets normalized by taking the difference with the average value for the expression of the tiles in the outmost tile offsets (tiles #1 and #9) represented based on the indicated color scale shown at the bottom.

FIG. 8. Pilot design analysis. (a) The same plot on average reporter expression by tile offset and group of tiled regions as in FIG. 2a but for the experiments conducted in K562 cells. (b) The fraction of reporter values that met the threshold at which 5% of outmost tile offsets (tiles #1 and #9) reporter values did for (left) HepG2 and (right) K562 cells experiments. (c) The same as (b) except just showing the range values broken down into four quartiles and showing in separate graphs replicate 1 (left) and replicate 2 (right). (d) Standard deviation (y-axis) of the reporter expression values at each tile offset (x-axis) for HepG2 experiments (left) and K562 experiments (right) for each replicate. (e) For the 100 HepG2 tiled regions selected to span a range of H3K27ac dip scores, receiver operating characteristic (ROC) curve for predicting regions with at least one tile above threshold, when ranking based on the dip score for the region for replicate 1 (left) and replicate 2 (right) experiments.

FIG. 9. Pilot correlation of reporter values as a function of distance. (a) Correlation of reporter expression values (y-axis) for each indicated offset (x-axis) both within (boxes) and across replicates (diamonds) in HepG2 (left) and K562 (right). (b) Cumulative number of adjacent large-step pairs of tiles (x-axis) that showed a difference in expression at a given uncorrected p-value (y-axis) based on a Mann-Whitney Test (see Methods) in HepG2 (left) experiments and K562 (right) experiments. (c) The false discovery rate (FDR) values corresponding to the p-values shown in (b).

FIG. 10. Pilot design motifs. (a) Motifs discovered based on running MEME software at an about 5% FDR value: (top) HepG2 activating, (center) HepG2 repressive, and (bottom) K562 activating 30-bp sequences extended by 10-bp to include the common sequence (see Methods). Up to the top four discovered motifs that had a reported E-value of 1 or less are shown. Motifs are ordered left to right as returned by the MEME software. There were no motifs discovered for the K562 negative set that met the criterion. Each discovered motif is matched to a best matching known motif using TOMTOM that is shown above the discovered motif (b) Motifs (rows) enriched in at least one of the four sets (HepG2 Activating, HepG2 Repressive, K562 Activating, and K562 Repressive) at least 2 fold based on the program extended to use a background of all nucleotides tested. The columns show the enrichment of the motif in the four sets considered using the same program and background. Motifs with at least three instances in the background are included, and, if multiple motifs corresponding to the same transcription factor met the threshold, the one with the highest enrichment is listed.

FIG. 11. Chromatin state model used for the scale-up experiments. (a) Emission probability matrix, showing expected frequency (percent) of each mark (columns) in each chromatin state (rows). For the scale-up experiments, a richer 25-state ChromHMM model is used to consider a more diverse set of chromatin states, whereas for the pilot analysis focused on strong enhancer states and low-activity states from a 15-state model (FIG. 7). (b) Average genome coverage (percent) and median fold enrichments as computed according to Gencode Transcription Start Sites (TSS)+/−2 kb; conserved elements using the SiPhy-PI method; ENCODE transcription factor binding data sets; and repression-associated nuclear lamina-associated domains. (c) Candidate state annotations. (d) Percent Genome Coverage of each state (rows) in six ENCODE cell types (columns). Shading highlights the cell types profiled here (HepG2, K562), and the cell types used to select the 15,720 tiled regions (HepG2, K562, HUVEC, H1-hESC). (e) TSS Enrichment (+/−2 kb of GENCODE 5′ Ends) for six ENCODE cell types. (f) State-to-State transition probabilities denoting the frequency (percent) with which state of the row transitions to the state of the column.

FIG. 12. Inference using multiple variance priors and for multiple replicates, promoters, and cell types, and effect of variance prior parameters on recovery of conserved elements. (a) Example inference with two different variance parameter priors. Effect of the variance prior on the output in the example in FIG. 3b right. If the variance prior is larger, the output can capture finer features, but can potentially overfit the data as seen by the activation peak on left unsupported by a transcription factor binding site prediction based on CENTIPEDE in close proximity to the repressive peak. On the other hand if a smaller variance prior is used, then the output is more consistently activating or repressive, but can underfit, providing a lower resolution output that can infer additional nucleotides as activating or repressive which are not. The strategy used was to apply the inference with both a relatively smaller variance prior (σ_{α}²=1) and a relatively larger variance prior (σ_{α}²=50) and merge the output of two inferences at each position, using the one with a smaller absolute value between the two when their signs agreed, and using 0 when their signs disagreed. (b) Summary of Sharpr-MPRA regulatory activity scores for each region. The resulting track of applying the two variance parameters is applied in six settings for each regulatory region, resulting in six tracks of Sharpr-MPRA scores, each of about 4.6 million nucleotides. The six tracks correspond to: (i,ii) one combined promoter (combinedP) track for each cell type (black), incorporating a total of four experiments per region, including both minP replicates and both SV40P replicates; (iii,iv) one track for the minimal promoter (minP) in each cell type (green), combining the two replicates for the minP promoter; and (v,vi) one track for the strong promoter (SV40P) in each cell type (blue), combining the two replicates for the SV40P promoter. In addition to these six tracks, SHARPR is applied to each replicate separately for each cell type, and the resulting inferences are used to evaluate the reproducibility of the inferences between individual replicates (FIG. 16a). For a subset of 44 genomic regions, two different barcode tilings overlaps perfectly, for all 295 nucleotides within them. For an additional 212 regions, multiple barcode tilings partially overlap, resulting in 256 regions with exact or partial overlap, of which 245 have two overlapping barcode sets, and 9 have three overlapping barcode sets. In forming the tracks, scores for a base were averaged when the base was overlapped multiple times. (c) For each indicated combination of low-variance prior parameter (row) and high-variance prior parameter (columns), ratio of the Area under the ROC curve (AUC) up to a 1% false positive rate (i-iii) or 5% false positive rate (iv-vi) for recovering SiPhy-PI conserved elements relative to what would be obtained by random guessing (AUC=0.00005 for 1% false positive rate; AUC=0.00125 for 5% false positive rate) for HepG2 (left) and K562 (right), when ranking nucleotides by: (i,iv) activating Sharpr-MPRA score; (ii,v) repressive scores; (iii,vi) absolute value of Sharpr-MPRA scores. Shading scale is specific to each heatmap. Boxes indicate the parameter combination used throughout the analyses (lower-variance σ_{α}²=1 and higher-variance σ_{α}²=50).

FIG. 13. Sharpr-MPRA regulatory activity score distribution. (a) Nucleotide-level score distribution for about 4.6 million nucleotide positions. The distribution of regulatory activity score at the nucleotide level for the combinedP promoter score (top row), minP score (middle row), and SV40P score (bottom row) in HepG2 cells (left) and K562 cells (right). The HepG2 distribution (top left) was also shown in FIG. 3. (b) MaxPos score distribution at the region level. Score distribution for all 15,720 tiled region, using the score of a single nucleotide for each, MaxPos, the nucleotide with maximum absolute activity score, shown based on the combined minP and SV40P data (top), minP (middle row), and SV40P (bottom), each for HepG2 (left) and K562 (right). Lighter shaded bars highlight the subset of regulatory regions with MaxPos ≥1 (primarily activating) and MaxPos ≤−1 (primarily repressive). (c) Absolute MaxPos, CenPos, and SymPos score distributions. Distribution of the absolute Sharpr-MPRA regulatory score (y-axis) for the 15,720 regions at the nucleotide position of maximum absolute score (MaxPos), the center nucleotide position (CenPos), and the symmetric nucleotide positions (SymPos), each ranked from highest to lowest absolute score (x-axis) for HepG2 (left) and K562 (right), using: combined minP and SV40P (combinedP) score (top row); the minP score (middle row); and the SV40P score (bottom row). Markings indicate the fraction of MaxPos nucleotides with absolute scores above the indicated values and are also listed for specific values based on the combinedP data (top). MaxPos, CenPos, and SymPos nucleotide positions are illustrated in FIG. 3b.

FIG. 14. Correlation between minP and SV40P inferred regulatory activity score by nucleotide position. (a) Correlation between the inferred regulatory activities based on the minimal and SV40P promoter data (y-axis) as a function of nucleotide position relative to the DNase peak center position in the tiling (x-axis) for HepG2 (left) and K562 (right). Higher correlation is observed closer to the center where each nucleotide is covered by more reporter constructs (FIG. 3a), and where more meaningful regulation is likely to occur (see FIG. 2a and FIG. 8). (b) Comparison of correlation (y-axis) between minP and SV40P using the same barcode set and using different barcode sets for 88 pairs of inferences in the 44 regions that were tiled twice using the same set of reporter sequences for HepG2 (left) and K562 (right).

FIG. 15. Nucleotide-level Sharpr-MPRA score correspondence at individual bases across promoter and barcode sets. (a) Comparison of Sharpr-MPRA regulatory activity score across minP and SV40 experiments for all nucleotide positions assigned to each indicated score range for HepG2 (left) and K562 (right), as shown by arrow in the diagram. For each row, left-most column indicates the score range, right-most column (# bases) indicates total number of nucleotide positions in that score range, and shaded columns indicate percentage of bases assigned to each score range bin. Each comparison was considered in both directions between promoter types, and each base is counted twice, once for its minP score, and once for its SV40P score. For example, about 83% of nucleotides with scores ≥1.5 in one promoter type show scores ≥1 in the other promoter type for HepG2 (about 71% for K562) (bottom boxes), and about 74% of nucleotides with scores <−0.5 in one promoter show scores <0 in the other promoter for HepG2 (about 73% for K562) (top boxes). (b) Same as (a) except restricted to those nucleotides overlapping a CENTIPEDE base in the cell type. (c) Comparison of Sharpr-MPRA scores at individual bases across different barcode sets for all 256 multiply tiled regions, including both exact and partial overlaps. Comparisons are shown for the minP and SV40P combined data (top), minP data (middle row), and SV40P data (bottom row), with format similar to panel in (a). (d) Same as (c) except for the subset of 44 regions that were covered by two sets of barcodes with exact tile overlap. (e) Comparison of Sharpr-MPRA scores inferred across different barcode sets for all 256 multiply tiled regions, including both exact and partial overlaps similar to (c), except restricted to those nucleotides overlapping a CENTIPEDE base in the cell type. (f) The same as (e), except for the subset of 44 regions that were covered by two sets of barcodes with exact tile overlap.

FIG. 16. Sharpr-MPRA within-region reproducibility correlation analysis. (a) Average within region Pearson correlation in activity scores across individual replicates of minP and SV40P experiments (y-axis) for regions meeting or exceeding varying maximum absolute score values (x-axis) for HepG2 (left) and K562 (right), with comparison performed indicated by the arrow in the corresponding diagram. Each region was included twice, once based on the MaxPos value from each replicate. Observed correlation is compared to the expected value and 95% confidence interval of 10,000 row-wise permutations of regions. Error bars indicate standard error. (b) Average Pearson correlation between minP and SV40P similar to panel in (a). Each region was included once and the MaxPos score on the x-axis is based on minP for the first row and SV40P for the second row. (c) Similar to (a,b), but with all comparisons performed across different barcode sets of the same experiment. Top: Barcode comparison using combined minP and SV40P data. Middle row: Barcode comparison using minP data. Bottom: Barcode comparison using the SV40P data. Each panel shows 88 points corresponding to two MaxPos values for each of the 44 regions with exact overlap tiling.

FIG. 17. Agreement in position of maximum absolute activity (MaxPos) between replicates, promoter types, and barcodes. (a) Average absolute distance (y-axis) between the position of maximum absolute activity (MaxPos) in one minP replicate and the other for those regions meeting or exceeding varying maximum absolute score values (x-axis) for HepG2 (left) and K562 (right). Each distance was included twice, once based on each of the two MaxPos values in the pair. This is compared to the expected distance between MaxPos in one minP replicate and a position in the other minP replicate if sampled randomly from the distribution of all MaxPos positions across all regions (RndMaxPos), and also compared to the distance between MaxPos in one minP replicate and the symmetric position in minP replicate (SymPos). This shows that even when MaxPos regions are highly off-centered, there is significantly higher agreement in the MaxPos position than expected by chance. (b) Same comparison as (a) for SV40P individual replicates for HepG2 (left) and K562 (right). (c) Same comparison as (a), but the comparisons are between SV40P and minP. (d) Same comparisons as (a), but comparing the combined minP and SV40P data for two different barcode sets for the 44 regions tiled with exact overlap from two barcode sets.

FIG. 18. Effect of the k-mer sequence within the 10-nucleotide barcode sequence on Sharpr-MPRA regulatory activity scores. (a) Cumulative distribution of the average inferred activity over each combination of k-mer sequence in the barcode, presence in each of the 31 tile positions, and the 295 inferred activity positions. Also shown are the expected and 95% confidence intervals based on 400 randomizations in which all the barcodes were randomly reassigned to reporter sequences. In all HepG2 plots, there is no visible difference between the observed, expected, and 95% confidence intervals. Arrows denote the visible differences where the observed distribution deviates from the expected distribution and the 95% confidence intervals. (b) Same as (a), but showing the difference between observed and expected data for each k-mer length and each cell type. Arrows denote the difference between the distribution of observed and expected activity for short k-mers in K562. In particular, AA and AT di-nucleotides were over-represented in the last two positions of barcodes with low reporter activity, indicating a technical bias in reverse transcription initiation, rather than a biological role of 3′ UTR motifs, as the last two barcode positions are the first to be reverse-transcribed. Note that the highlighted differences are on the order of 0.01, compared to activity scores ranging between 0.5-8 for positions that show motif enrichments (see for example FIG. 3c, or FIG. 19).

FIG. 19. Overlap with CENTIPEDE predicted transcription factor binding sites as a function of Sharpr-MPRA regulatory activity score. Extended version of FIG. 3c left, but showing the results for both K562 and HepG2 and the combined data, minP data, and SV40P data. Each point represents the average of 927 nucleotide positions in each of 5,000 quantiles. Horizontal dark line shows the expected overlap averaged across all 295 nucleotide positions of each region, and the lighter shaded line shows the expected overlap fraction at the center nucleotide position. These are shown for: (a) the combined minP and SV40P data for HepG2 (left) and K562 (right). (b) The minP data for HepG2 (left) and K562 (right). (c) The SV40P data for HepG2 (left) and K562 (right).

FIG. 20. Individual examples. (a) The relative Sharpr-MPRA regulatory activity scores overlapping all cell type specific binding sites for GABPA in HepG2 cells predicted based on the CENTIPEDE method in regions tested. Shown are the GABPA motifs, and predicted binding sites in HepG2 for other regulators. The Sharpr-MPRA score based on the SV40P data is shown, the minP data is shown, and the combination of SV40P and minP is shown. (b) Same as (a) except for the predicted binding sites of the repressor REST in HepG2 cells. (c,d) The same as (a,b) except for K562 cells.

FIG. 21. Comparison of different footprint sets. The plot evaluates in K562 cells the average absolute regulatory activity (y-axis) for positions overlapping predictions of locations of transcription factor binding sites predicted based on DNase footprint information in K562 cells by five different methods. Two of the five methods also use motif information, CENTIPEDE and PIQ, while the other three methods are motif independent, Wellington and the methods of Boyle et al. and Neph et al. The x-axis shows the fraction of nucleotides for which each of these footprint sets overlap, showing the two footprint sets that overlap more nucleotides had a relatively lower average absolute activity compared to the other three. All five sets had greater absolute activity than four different baselines: (1) at the center position restricted to regions overlapping a K562 DNase peak, (2) at any position overlapping a K562 DNase peak, (3) at the center position over all regions, and (4) over all positions in all regions tested.

FIG. 22. Overlap with motif instance predictions as a function of Sharpr-MPRA regulatory activity score. These are analogous plots to FIG. 19 except the plots are shown for the set of nucleotides covered by a motif instance prediction, which does not use conservation or make cell type specific predictions. The plot is for Sharpr-MPRA regulatory activity scores in HepG2 (left) and K562 (right) cells based on: (a) minP and SV40P combined data; (b) minP data; and (c) SV40P data.

FIG. 23. Overlap with conserved elements as a function of Sharpr-MPRA regulatory activity score. Extended version of FIG. 3c and analogous to FIG. 19, but showing the results for bases overlapping conserved elements from the SiPhy-PI method for HepG2 cells (left) and K562 cells (right), based on: (a) the combined minP and SV40P data; (b) minP data; and (c) SV40P data.

FIG. 24. Comparison with DeltaSVM predictions. (a) Overlap with nucleotide positions predicted by DeltaSVM to contain top 1% Negative Mutations to Reference Sequence. Analogous plots to FIG. 3c, and FIGS. 19 and 22-23, but the enrichment is based on the 1% nucleotide positions tested that are predicted by DeltaSVM to have a possible mutation to the reference sequence that would cause the greatest decrease in regulatory activity (see Methods). Overlaps are shown for HepG2 (left) and K562 (right) for the combined SV40P and minP data (top row), minP data (middle row), and SV40P data (bottom row). An enrichment for DeltaSVM predictions is seen for nucleotides among the most activating predicted bases, but not the most repressive. (b) Overlap with nucleotides predicted by DeltaSVM to contain top 1% Positive Mutations to the Reference Sequence. Analogous to (a), but based on the top 1% nucleotides having a possible mutation to the reference sequence leading to the greatest predicted increase in regulatory activity (see Methods). Overlap is shown for HepG2 (left) and K562 (right), for the combined minP and SV40P data (top row), the minP data (middle row), and the SV40P data (bottom row). A depletion is seen in the most activating predicted bases.

FIG. 25. Enrichment based on maximum absolute activity position. These are similar plots to those shown in FIG. 3c and FIGS. 19 and 23 except the scatter plots are based on the single position which had the highest absolute Sharpr-MPRA regulatory activity score in each region tested (MaxPos nucleotides). The sign of the score is preserved for the analysis. In these plots there are 200 quantiles, so each point corresponds to about 79 unique MaxPos nucleotides. The lighter shaded horizontal line represents the overlap fraction based on the center position and the dark horizontal line represents overall overlap fraction of MaxPos nucleotides. The plots are (a) for transcription factor binding sites predicted by the CENTIPEDE method for HepG2 (left) and K562 (right) and (b) for conserved elements detected by the SiPhy-PI method for HepG2 (left) and K562 (right).

FIG. 26. Sharpr-MPRA activity score and standard deviation by position. (a) Top row: Central positions show higher average absolute activity score for HepG2 (left) and K562 (right) for the minP data, SV40P data, and combinedP data. Middle row: Central positions also show higher standard deviation of activity score. Bottom row: Average signed activity does not show a bias for central positions, when averaging both positive and negative values. (b) Distribution of the location of maximum absolute regulatory activity (MaxPos) in each region among the 59 five-nucleotide intervals, for HepG2 (left) and in K562 (right).

FIG. 27. Ranked MaxPos overlap with CENTIPEDE motifs and evolutionarily-conserved nucleotides. (a) The three-way comparison of the cumulative overlap (y-axis) with CENTIPEDE predicted transcription factor binding sites analogous to FIG. 3d (left panel) for HepG2, and shown here also for K562 for center nucleotide positions (CenPos), maximum-absolute-score nucleotide positions (MaxPos), and their symmetric nucleotide positions (SymPos), each ranked from highest to lowest based on the absolute Sharpr-MPRA scores (x-axis) for the (top) combinedP data, (middle) minP data, and (bottom) SV40P data indicating that the inference strategy captures regulatory nucleotides at high resolution. MaxPos, CenPos, and SymPos nucleotide positions are illustrated in FIG. 3b. (b) Overlap with conserved elements predicted by the SiPhy-PI method analogous to FIG. 3d (right panel) for HepG2, and shown here also for K562 for center nucleotide positions (CenPos), maximum-absolute-score nucleotide positions (MaxPos), and their symmetric nucleotide positions (SymPos), each ranked from highest to lowest based on the absolute Sharpr-MPRA scores (x-axis) for the (top) combinedP data, (middle) minP data, and (bottom) SV40P data. MaxPos, CenPos, and SymPos nucleotide positions are illustrated in FIG. 3b. The plots indicate that the inference strategy captures functional nucleotides at high resolution. The MaxPos nucleotides have higher overlap with conserved elements than CenPos nucleotides except at low absolute activity scores (see FIG. 13c).

FIG. 28. Impact of number of tiles and tiling interval on motif and conserved element recovery. Recovery of (a) CENTIPEDE motif instances and (b) evolutionarily-conserved elements from the SiPhy-PI method based on the AUC up to a false positive rate (FPR) of 5% (y-axis), as a function of the number of tiles (x-axis), when ranking nucleotides by absolute Sharpr-MPRA regulatory activity score in HepG2 (left) and K562 (right). Multiple points for the same vertical line correspond to different step sizes leading to the same number of tiles within the 295 bp region tested.

FIG. 29. Impact of number of tiles and tiling interval on correlation. Correlation between the minP and SV40P experiments (y-axis) at different positions relative to the center (x-axis) for regulatory activity inferred using a subset of tiles selected by varying the step size for HepG2 (top) and K562 (bottom). In all cases the center tile is retained. If two or more step sizes led to the same number of tiles within the 295 bp region tested, then the correlations based on the smallest step size is plotted. It is found that increasing the number of tiles (e.g., decreasing the step size) leads to increased correlation levels supporting the high-density tiling approach.

FIG. 30. (a) Motif average Sharpr-MPRA regulatory score concordance between minP and SV40P data. Scatter plot of the motif average Sharpr-MPRA regulatory obtained by averaging the activity at the central motif position for all motif instances using the minP scores (x-axis) or the SV40P scores (y-axis), for HepG2 (left) and K562 (right). Correlation between minP and SV40P motif scores is about 0.98 in HepG2 and about 0.95 in K562. (b) Motif average Sharpr-MPRA regulatory score remains unchanged when using the high-variance prior parameter. Average motif regulatory score using the larger-variance (σ_{α}²=50) prior parameter (x-axis) versus combining inferences from both a lower-variance (σ_{α}²=1) and higher-variance (σ_{α}²=50) prior parameters (y-axis, same as FIG. 4a) for HepG2 (left) and K562 (right). Each point corresponds to one motif. Motifs with >10 instances are shown. In principle, the use of a larger-variance parameter could potentially allow better recovery of regulatory motifs, as it would allow scores to locally become higher for activating motifs, and locally become lower for repressive motifs. In practice however, using the larger-variance parameter did not affect motif scores (correlation >about 0.99 for both HepG2 and K562), and thus may not have a significant effect on the recovery of functional motifs.

FIG. 31. Top differential motifs and motifs with most significant activation or repression enrichment. (a,b) For both panels the column headers are: b, Motif logo and name (TF family and id within family); motif names with a “disc” were based on de novo discovery in ENCODE ChIP-seq data; c, cell type of most significant enrichment (Act and Rep Enr columns); d-e, classification as Activating (Act, −log₁₀P ≥2), Repressive (Repr, −log₁₀P ≥2), Dual (both), Neither (neither) based on Act and Rep enrichment (Enr); f, total number of motif instances; g-i, average combinedP score at center motif position in HepG2 and K562 and their difference; k, t-test corrected P-value of difference in activity; m, −log₁₀P of t-test corrected value (for 1934 motifs); n-q, −log₁₀P of enrichment in activating (Act, activity ≥1) or repressive (Repr, activity ≤−1) nucleotides for HepG2 and K562; r-s, difference in activating (Act) or repressive (Repr) in terms of −log₁₀P of enrichment (more activating/repressive in HepG2, or more activating/repressive in K562); t-w, number of instances selected based on chromatin data in the cell type tested and the average activity of those instances for HepG2 and then K562. Top-ranked member of each “TF group” is shown. (a) Motifs with most significant activity difference between HepG2 and K562 (Pdiff). Top-ranked motifs determined by t-test of difference in activity. Display Cutoff: t-test corrected p-value ≤0.05. (b) Motifs with most significant (P<0.01) enrichment in activating (score ≥1) or repressive (≤−1) bases in K562 or in HepG2. Rows sorted by maximum absolute activity enrichment of n-q (af). Columns reordered to match the information highlighted. N/S denotes difference between HepG2 and K562 scores not significant (corr. P >0.05).

FIG. 32. Scatterplot of regulatory motif enrichments. (a) comparing the −log 10 P-value of the enrichment for regulatory motif instances with activity ≥1 at the center position in HepG2 (y-axis) and in K562 (x-axis) for all regulatory motifs shown in FIG. 31. (b) Same as (a) except considering repression based on enrichment for regulatory motif instances with activity ≤−1.

FIG. 33. Sharpr-MPRA regulatory score aggregation plots relative to motif position. Aggregation plots of Sharpr-MPRA regulatory scores relative to motif position from FIG. 4b shown separately for instances whose motif center fell within the central 51 bp, or to the left or right in HepG2 (left) and K562 (right) cells, shown for: (a) an ETS motif; (b) a HNF4 motif; (c) a GATA motif; (d) a REST motif; and (e) a RFX motif, which predicted motif instances independent of cell type. Vertical error bars indicate standard error.

FIG. 34. 7-mer Sharpr-MPRA regulatory activity score plot. The plot has a point for each 7-mer appearing more than ten times based on the forward strand showing the average regulatory activity score in HepG2 cells (x-axis) and the average regulatory activity score in K562 cells (y-axis).

FIG. 35. Enrichment for regulatory motif instances in top 5% activated and top 5% repressed nucleotides. These are similar to the plots shown in FIG. 4c except the activating and repressive bases are specified as the 5% of nucleotides that were given the most activating and repressive scores respectively instead of using the 1 and −1 thresholds. Activator score (y-axis) and repressor score (x-axis) for a compendium of regulatory motifs (points) in HepG2 (left) and K562 (right), based on the statistical significance (−log₁₀P) for the enrichment of the center position of motif instances in positions that had a regulatory score in the lowest 5% (most repressive) or highest 5% (most activating), computed based on one-side binomial tests where the probability of success for the binomial distribution is the fraction of total nucleotides tested that met the threshold. Motif instances that appeared on both strands at the same position are counted once. (a,b) The scatter plots are for (a) all points in HepG2 and (b) a zoomed-in view of the points in HepG2 that have values less than 20 on both axes. (c,d) are similar except for K562.

FIG. 36. Regulatory activity in repeat classes and families. Extended set of enrichments shown in FIG. 5 now showing in each column for (a) ERV1, (b) LINE, (c) LTR, (d) SINE, (e) DNA, (f) ERVL, (g) ERVL-MALR, (h) ERVK, (i) Simple Repeat, and (j) Low Complexity repeats as specified by RepeatMasker for regulatory activity scores from top to bottom: HepG2 combinedP, HepG2 minP, HepG2 SV40P, K562 combinedP, K562 minP, and K562 SV40P data. Bins were formed by assigning each base to the nearest 0.5 value based on its regulatory score, and the two extreme bins contain all more extreme values. Lighter shaded line denotes the expected fraction of overlap based on the center position (CenPos), and the darker line denotes the expected fraction based on all positions.

FIG. 37. Activity by chromatin state—K562 cells. Analogous figures as FIG. 6a-b except for K562 cells. (a) For each chromatin state (x-axis) the average K562 regulatory score of all MaxPos nucleotides in K562 is shown, over all regions selected based on that state in that cell type. Results are shown for the combinedP, minP, and SV40P results (consecutive bars). The number of regions selected based on each state in the cell type is shown on the bottom. Since the regions were selected based on the indicated cell type, the regions all have DNase sites. (b) For each chromatin state, the average score at MaxPos nucleotides for regions with the center position in the state based on all locations tested is shown separately for locations overlapping DNase sites and those not overlapping DNase sites in K562. The number of regions in each set is indicated along the bottom. The vertical error bar indicates one standard error.

FIG. 38. Fraction of regions showing above activating or below repressive threshold at the maximum absolute score position (MaxPos). (a) For each chromatin state (rows) in HepG2 (left panel) and K562 (right panel), the number of regions tested (“All”) that were selected based on the chromatin state annotation in the same cell type (DNase Matched), the percentage of those regions with score at MaxPos ≥1 (“Act” for “Activating”), ≤−1 (“Rep” for “Repressive”), or neither (“Non”) using the combinedP score (first column group), minP score (second group), or SV40P score (third group). Each individual column is shaded based on median, the 90^{th} percentile, and the 10^{th} percentile, thus indicating chromatin states that are more often activating (e.g., Tss, Enh) or repressive (e.g., ReprD, Repr) than expected on average. Dark boxes highlight the numbers discussed in the main text. (b) Similar to (a), but for DNase sites that were selected in a different cell type and are also DNase in the tested cell type. (c) Same as (a), but showing total counts and activity for all DNase in sites, regardless of the cell type in which they were selected. Total region counts (“All”) are the sum of panels (a) and (b). Dark boxes highlight the numbers discussed in the main text. (d) Same percentages but shown for the converse set regions, namely all the regions that do not show DNase activity in the tested cell type (and thus “Unmatched”, as all regions selected were DNase in the cell type in which they were selected). Differences in the fraction of active regions between (a,b,c) versus (d) reflect that among all regions in a given chromatin state, those that also overlap DNase regions are more likely to be activating (see also FIG. 6b and FIG. 37b). (e) Same as (a-d), but showing all tested regions, regardless of which cell type they were selected, and regardless of whether they are found in a DNase region or a non-DNase region. This shows that active chromatin states (e.g., Tss, Enh) are more likely to show activating regulatory scores (≥1), and that repressive chromatin states (e.g., Repr, ReprD, ReprW) are more likely to show repressive regulatory scores (≤−1).

FIG. 39. Cumulative distribution of region activity scores for each chromatin state. Cumulative fraction of regions (y-axis) showing a MaxPos activity score greater than indicated (on the x-axis) for each chromatin state (see FIG. 11) based on the combinedP score (top row), minP score (middle row) and SV40P score (bottom row) for HepG2 (left column) and K562 (right column), and shown for: (a) all selected regions from all four cell types that are DNase regions in the tested cell type (N=5840 in HepG2, N=6682 in K562); (b) the subset of DNase regions in the tested cell type that were selected in that cell type (N=3930 in each of HepG2 and K562); and (c) all DNase regions selected in another cell type, that were not in a DNase region in the tested cell type (N=9880 in HepG2, N=9038 in K562). Each region is shown as a separate point (plus signs). Arrows highlight the active promoter and enhancer states (1_Tss, 5_Enh) which had the greatest fraction of regions with strongly activating MaxPos scores, and the single-cut DNase state (10_DNaseD) which had an increased presence among regions with the most repressive MaxPos scores.

FIG. 40. CRE-sequence activity distribution for ChromHMM chromatin states conditioned on presence or absence of DNase. Average CRE-sequence expression data in K562 (y-axis) partitioned by ChromHMM state in K562 (see FIG. 11) and by DNase versus non-DNase based on the center position of tested segments. Another study reported lower CRE-sequence activity for H3K27ac-marked enhancer regions, and higher activity for non-H3K27ac enhancer regions, contrary to the results here. As FIG. 40 shows, this reversal in effect is partly explained by the mixing together of two different classes of regions (DNase vs. non-DNase), whose representation was highly unbalanced between the two groups compared, a statistical effect referred as Simpson's Paradox. 80 of 212 H3K27ac-marked enhancer regions tested captured DNase sites, compared to 174 of 255 non-H3K27ac enhancer regions. When the tested regions are separated by both chromatin state and by DNase overlap, it is found that regions overlapping DNase sites show higher median CRE-sequence activity than non-DNase regions within the same chromatin state (as in FIG. 6b and FIG. 37b), and that H3K27ac-marked enhancer regions show higher median activity than non-H3K27ac enhancer regions within DNase regions (as in FIG. 6a and Supplementary FIG. 37a). This emphasizes the notion of centering tested elements on candidate driver nucleotides (e.g., regulatory motifs, H3K27ac dips, DNase peaks). This is especially of note when testing short elements without tiling (130-bp in the case of CRE-seq), as positioning the elements without such evidence may exclude driver regulatory nucleotides from tested regions, which can have a strong effect on reporter activity. All states with >5 regions tested are shown. Boxplots generated with Matlab boxplot command.

FIG. 41. In vivo TF binding is higher and more centrally positioned for higher-scoring regions in pilot experiments. (a, b) Number of HepG2 ENCODE TF binding ChIP-Seq peak calls out of 77 sets (y-axis) overlapping regions with higher reporter scores (above threshold) and lower reporter scores (below-threshold) for each nucleotide position relative to the selected region center (x-axis) for: (a) the 100 regions tested in HepG2 and selected to have the highest in vivo dip scores in HepG2; (b) and the 100 regions selected to represent a range of dip scores in HepG2 cells. (c) The same plot as in (a, b) except for the 50 regions selected based on being in an active enhancer in K562 cells and for the 150 ENCODE peak call sets from K562 cells. Reporter activity threshold selected at the 95^{th} percentile of outmost tile offsets. Vertical error bars indicate one standard error. These plots indicate that regions with higher reporter activity show higher TF binding within the tested region (higher peak in the center), and more concentrated TF binding (lower peaks in the surrounding) compared to lower reporter activity regions.

FIG. 42. In vivo TF binding is higher and more centrally positioned for activating regions in active regulatory chromatin states for scale-up experiments. Number of HepG2 (left) and K562 (right) ENCODE TF binding ChIP-Seq peak calls (y-axis) overlapping regions with activating regulatory scores (combinedP MaxPos score ≥1), repressive regulatory scores (combinedP MaxPos score ≤−1) or neither for each position relative to the selected region center (x-axis) for DNase regions selected in the matched cell type where they were tested (DNase matched) for: (a) 200 active promoter states (1_Tss) per cell type; (b) 1200 strong enhancer states (5_Enh); and (c) 600 weak enhancer states (8_EnhW). Vertical error bars indicate one standard error. These plots indicate that regions with higher regulatory activity show higher TF binding within the tested region (higher peak in the center), and more concentrated TF binding (lower peaks in the surrounding) compared to lower regulatory activity regions.

FIG. 43. Scatter plot relationship between region activity and distance to nearest DNase site for selected chromatin states. (a) Scatter plot showing, for each tiled region selected based on being in a DNase site in promoter state 1_Tss in HepG2 cells on the x-axis, the log₁₀ distance in bp to the nearest DNase site in HepG2 (excluding itself) and on the y-axis the Sharpr-MPRA regulatory activity score at the MaxPos nucleotide for the region based on the HepG2 combinedP data. Also shown is a line of best fit for the scatter plots. (b) The same as (a) except for K562 cells. (c, d) The same as (a, b) except for enhancer state 5_Enh. (e, f) The same as (a, b) except for weak enhancer state 8_EnhW. The plots show for these states that more isolated DNase sites tend to have greater positive activity. The correlation for all states are shown in FIG. 44.

FIG. 44. Correlation of each chromatin state for region activity and distance to nearest DNase site. Bar graph showing for the regions selected based on each of the 25 chromatin states the correlation between the log of the distance to the nearest DNase site in that cell type (excluding itself) and the regulatory activity score at the MaxPos nucleotide for HepG2 chromatin states (left) and K562 chromatin states (right), using: (a) the combined minP and SV40P score; (b) the minP data; and (c) the SV40P data. Full scatter plot for selected states are found in FIG. 43.

FIG. 45. 95% Confidence interval bounds for expected motif regulatory activity score averages based on chromatin context. (a) Scatter plots analogous to FIG. 6c showing the observed average regulatory activity (y-axis) compared with the lower bound of a 95% confidence interval (2.5^{th} percentile) on the motif regulatory score expected based on the chromatin context (x-axis) for HepG2 (left) and K562 (right). (b) Same as (a) except the x-axis represents the upper bound on a 95% confidence interval (97.5^{th} percentile).

FIG. 46. A computing device implemented in accordance with some embodiments of this disclosure.

### DETAILED DESCRIPTION

Some embodiments of this disclosure are directed to a method of determining regulatory activity of nucleotides in a genome, which includes: (1) designating a reference region of the genome; (2) designating a plurality of reporter tiles covering the reference region, where each reporter tile has a length L, and the reporter tiles are offset from one another with a step size s; and (3) generating a plurality of reporter constructs corresponding to the reporter tiles.

In some embodiments of the method, the reference region is a potential regulatory region, or includes a potential regulatory sequence of nucleotides.

In some embodiments of the method, a ratio of s to L is about 1:4 or less, about 1:5 or less, about 1:6 or less, about 1:7 or less, about 1:8 or less, about 1:9 or less, about 1:10 or less, about 1:15 or less, about 1:20 or less, about 1:25 or less, or about 1:29 or less. In some embodiments of the method, L is about 50 bps or more, about 75 bps or more, about 100 bps or more, about 125 bps or more, about 145 bps or more, about 150 bps or more, about 175 bps or more, about 200 bps or more, or about 225 bps or more, such that the reporter tiles are each L in length. In some embodiments of the method, s is about 60 bps or less, about 55 bps or less, about 50 bps or less, about 45 bps or less, about 40 bps or less, about 35 bps or less, about 30 bps or less, about 25 bps or less, about 20 bps or less, about 15 bps or less, about 10 bps or less, or about 5 bps or less. In some embodiments of the method, N=L/s denotes a number of nucleic acid intervals of the step size s covered by each reporter tile, where N is about 4 or more, about 5 or more, about 6 or more, about 7 or more, about 8 or more, about 9 or more, about 10 or more, about 15 or more, about 20 or more, about 25 or more, or about 29 or more. In some embodiments of the method, L is divisible by s, such that N is an integer. In some embodiments of the method, a number J of the reporter tiles cover the reference region, such that the reference region is tiled with the J reporter tiles, and J is about 5 or more, about 7 or more, about 9 or more, about 11 or more, about 14 or more, about 16 or more, about 18 or more, about 20 or more, about 23 or more, about 25 or more, about 29 or more, or about 31 or more.

In some embodiments of the method, generating the reporter constructs is performed using microarray-based nucleic acid synthesis. In some embodiments of the method, each reporter construct includes a corresponding reporter tile of the reporter tiles, or includes a nucleic acid sequence matching a nucleic acid sequence of the corresponding reporter tile. In some embodiments, each reporter construct also includes a distinct, identification nucleic acid barcode or tag, such that a distinct barcode is paired with each corresponding reporter tile. By “reporter construct” of some embodiments, reference is made to an artificial (e.g., not naturally occurring) continuous sequence of nucleotides.

In some embodiments of the method, the method further includes inserting the reporter constructs into expression vectors, where the resulting expression vectors each includes at least one of the reporter constructs; introducing the resulting expression vectors into cells in which the barcodes of the reporter constructs are expressed; and determining extents of the barcodes expressed in the cells, where an extent of each barcode expressed is an indication of a regulatory activity of a corresponding reporter tile. In some embodiments, determination of an extent of expression of a barcode can be performed by measurements obtained from quantitatively sequencing nucleic acid molecules resulting from cDNA synthesis or determining a quantity of mRNA hybridized to nucleic acid molecules complementary to the barcode. By “expression vector” of some embodiments, reference is made to a nucleic acid that includes an open reading frame and, when introduced into a host cell, includes nucleic acid components to allow mRNA expression of the open reading frame. An “expression vector” of some embodiments also include components for replication and propagation of the vector in a host cell.

In some embodiments of the method, operations (1), (2), and (3) are performed across multiple regions of the genome, where a number of the regions is about 10 or more, about 50 or more, about 100 or more, about 150 or more, about 200 or more, about 250 or more, about 500 or more, about 1000 or more, about 5000 or more, about 10000 or more, or about 15000 or more.

More generally, in some embodiments, the reference region has a center position, and the reporter tiles cover the reference region with respect to the center position. The reporter tiles can be centered with respect to the center position, such that a number of the reporter tiles downstream of the center position is the same as a number of the reporter tiles upstream of the center position. The reporter tiles also can be off-centered with respect to the center position, such that a number of the reporter tiles downstream of the center position is different from a number of the reporter tiles upstream of the center position. The reference region is tiled with the reporter tiles with overlaps to allow for a number of sequential or continuous bps in common in adjacent reporter tiles. An overlapping number of sequential bps can be the same for each adjacent pair of the reporter tiles. For example, the overlapping number of sequential bps can be less than L and denoted as L−s, with L and s denoted as above. The overlapping number of sequential bps can be at least a majority of the length L of the reporter tiles, and can be represented as a fraction 1−(s/L) of the length L, such as about 3/4 or more, about 4/5 or more, about 5/6 or more, about 6/7 or more, about 7/8 or more, about 8/9 or more, about 9/10 or more, about 14/15 or more, about 19/20 or more, about 24/25 or more, or about 28/29 or more. The overlapping number of sequential bps also can differ among adjacent pairs of the reporter tiles. The reporter tiles can have the same or a different number of bps in length.

Additional embodiments of this disclosure are directed to a series of reporter constructs for determining regulatory activity of nucleotides in a genome, the series of reporter constructs includes a plurality of reporter constructs, each of which includes (1) an identification nucleic acid barcode or tag and (2) a nucleic acid sequence corresponding to a reporter tile of a plurality of reporter tiles. The reporter tiles cover a reference region of the genome, each reporter tile has a length L, and the reporter tiles are offset from one another with a step size s.

In some embodiments of the series of reporter constructs, the reference region is a potential regulatory region, or includes a potential regulatory sequence of nucleotides.

In some embodiments of the series of reporter constructs, a ratio of s to L is about 1:4 or less, about 1:5 or less, about 1:6 or less, about 1:7 or less, about 1:8 or less, about 1:9 or less, about 1:10 or less, about 1:15 or less, about 1:20 or less, about 1:25 or less, or about 1:29 or less. In some embodiments of the method, L is about 50 bps or more, about 75 bps or more, about 100 bps or more, about 125 bps or more, about 145 bps or more, about 150 bps or more, about 175 bps or more, about 200 bps or more, or about 225 bps or more, such that the reporter tiles are each L in length. In some embodiments of the method, s is about 60 bps or less, about 55 bps or less, about 50 bps or less, about 45 bps or less, about 40 bps or less, about 35 bps or less, about 30 bps or less, about 25 bps or less, about 20 bps or less, about 15 bps or less, about 10 bps or less, or about 5 bps or less. In some embodiments of the method, N=L/s denotes a number of nucleic acid intervals of the step size s covered by each reporter tile, where N/s about 4 or more, about 5 or more, about 6 or more, about 7 or more, about 8 or more, about 9 or more, about 10 or more, about 15 or more, about 20 or more, about 25 or more, or about 29 or more. In some embodiments of the method, L is divisible by s, such that N/s an integer. In some embodiments of the method, a number J of the reporter tiles cover the reference region, such that the reference region is tiled with the J reporter tiles, and J is about 5 or more, about 7 or more, about 9 or more, about 11 or more, about 14 or more, about 16 or more, about 18 or more, about 20 or more, about 23 or more, about 25 or more, about 29 or more, or about 31 or more.

In some embodiments of the series of reporter constructs, the reporter constructs include a first reporter construct which includes a first identification nucleic acid barcode and a first nucleic acid sequence corresponding to a first reporter tile of the reporter tiles, a second reporter construct which includes a second identification nucleic acid barcode and a second nucleic acid sequence corresponding to a second reporter tile of the reporter tiles, a third reporter construct which includes a third identification nucleic acid barcode and a third nucleic acid sequence corresponding to a third reporter tile of the reporter tiles, and so on up to a J^{th} reporter construct which includes a J^{th} identification nucleic acid barcode and a J^{th} nucleic acid sequence corresponding to a J^{th} reporter tile of the reporter tiles. The identification barcodes of the reporter constructs are different from one another. The first nucleic acid sequence and the second nucleic acid sequence have an overlapping number L−s of sequential bps in common, the second nucleic acid sequence and the third nucleic acid sequence have the overlapping number L−s of sequential bps in common, and so on up to the (J−1)^{th} nucleic acid sequence and the J^{th} nucleic acid sequence have the overlapping number L−s of sequential bps in common.

In some embodiments of the series of reporter constructs, included are a first plurality of reporter constructs for a first region of the genome, a second plurality of reporter constructs for a second region of the genome, a third plurality of reporter constructs for a third region of the genome, and so on across multiple regions of the genome, where a number of the regions is about 10 or more, about 50 or more, about 100 or more, about 150 or more, about 200 or more, about 250 or more, about 500 or more, about 1000 or more, about 5000 or more, about 10000 or more, or about 15000 or more.

Additional embodiments of this disclosure are directed to a population of cells including the reporter constructs of any of the foregoing embodiments.

Additional embodiments of this disclosure are directed to a method of analyzing and mapping regulatory activity of nucleotides in a genome, which includes: (1) providing or receiving measurements of activities of a plurality of reporter tiles covering a reference region of the genome, where each reporter tile has a length L, the reporter tiles are offset from one another with a step size s, and each reporter tile covers L/s nucleic acid intervals of the step size s; (2) providing or generating a probabilistic model relating the activities of the reporter tiles to activities of nucleic acid intervals of the step size s within the reference region; and (3) using the probabilistic model, deriving an activity of each nucleic acid interval of the step size s within the reference region.

In some embodiments of the method, the reference region is a potential regulatory region, or includes a potential regulatory sequence of nucleotides.

In some embodiments of the method, L is about 50 bps or more, about 75 bps or more, about 100 bps or more, about 125 bps or more, about 145 bps or more, about 150 bps or more, about 175 bps or more, about 200 bps or more, or about 225 bps or more, such that the reporter tiles are each L in length. In some embodiments of the method, s is about 60 bps or less, about 55 bps or less, about 50 bps or less, about 45 bps or less, about 40 bps or less, about 35 bps or less, about 30 bps or less, about 25 bps or less, about 20 bps or less, about 15 bps or less, about 10 bps or less, or about 5 bps or less. In some embodiments of the method, the number L/s=N of nucleic acid intervals of the step size s covered by each reporter tile is about 4 or more, about 5 or more, about 6 or more, about 7 or more, about 8 or more, about 9 or more, about 10 or more, about 15 or more, about 20 or more, about 25 or more, or about 29 or more. In some embodiments of the method, L is divisible by s, such that N is an integer. In some embodiments of the method, a number J of the reporter tiles cover the reference region, such that the reference region is tiled with the J reporter tiles, and J is about 5 or more, about 7 or more, about 9 or more, about 11 or more, about 14 or more, about 16 or more, about 18 or more, about 20 or more, about 23 or more, about 25 or more, about 29 or more, or about 31 or more. In some embodiments, a total number of the nucleic acid intervals of the step sizes within the reference region is denoted as N+(J−1).

In some embodiments of the method, the probabilistic model relates an activity of each reporter tile to activities of L/s nucleic acid intervals of the step size s covered by the reporter tile.

In some embodiments of the method, the method further includes deriving an activity of each nucleotide within the reference region from the activities of the nucleic acid intervals of the step size s within the reference region. In some embodiments of the method, deriving the activities of the nucleotides within the reference region includes performing piecewise linear interpolation of the activities of the nucleic acid intervals of the step size s within the reference region. In some embodiments, the method further includes determining whether a sequence of nucleotides within the reference region is activating, or repressive, or both.

In some embodiments of the method, the method is computer-implemented such that one or more of operations (1), (2), and (3) are performed by a processor, and the probabilistic model is stored in a memory connected to and accessible by the processor.

In some embodiments of the method, data from which activities are derived are normalized. In some embodiments, derived activity is cell-type specific. In some embodiments, activities are derived using both a low-variance prior and a high-variance prior.

Further embodiments of this disclosure are directed to a non-transitory computer-readable storage medium including instructions stored therein, that upon execution by a processor of a computing device, direct the processor to perform operations of the method of any of the foregoing embodiments.

### Example

The following example describes specific aspects of some embodiments of this disclosure to illustrate and provide a description for those of ordinary skill in the art. The example should not be construed as limiting this disclosure, as the example merely provides specific methodology useful in understanding and practicing some embodiments of this disclosure.

Massively parallel reporter assays (MPRAs) allow nucleotide-resolution dissection of transcriptional regulatory regions, such as enhancers, but just few regions at a time. Herein disclosed is a combined experimental and computational approach, systematic high-resolution activation and repression profiling with reporter tiling using MPRA (Sharpr-MPRA), that allows high-resolution analysis of thousands of regions simultaneously. Sharpr-MPRA combines dense tiling of overlapping MPRA constructs with a probabilistic graphical model to recognize functional regulatory nucleotides, and to distinguish activating and repressive nucleotides, using their inferred contribution to reporter gene expression. Sharpr-MPRA is used to test about 4.6 million nucleotides spanning about 15,000 putative regulatory regions tiled at 5-nucleotide resolution in two human cell types. The results recovered cell-type-specific regulatory motifs and evolutionarily conserved nucleotides, and distinguished activating and repressive motifs. The results also showed that endogenous chromatin state and DNA accessibility are both predictive of regulatory function in reporter assays, identified retroviral elements with activating roles, and uncovered “attenuator” motifs with repressive roles in active chromatin.

As disclosed herein, challenges of using dense tiling of MPRA constructs and computational analysis to infer activating and repressive nucleotides at high resolution across many regions are overcome. The combined approach, Sharpr-MPRA, and the associated computational method is termed SHARPR. Sharpr-MPRA is used to dissect over about 15,000 putative regulatory regions from genome-wide epigenomic maps. Each 295-base-pair (bp) region at 5-nucleotide offsets is tiled using overlapping 145-nucleotide constructs. About 4.6 million nucleotide inferences are made, each in two cell types, and distinguished activating and repressive regulatory functions without the use of motifs or other sequence information. Inferred regulatory nucleotides were reproducible, high-resolution, cell-type-specific and supported by evolutionary conservation and regulatory motif evidence. The strategy provided gene-regulatory insights, including activating motifs lacking well-established regulators, “dual-role” motifs with both activating and repressive roles, strongly activating repeat elements and “attenuator” motifs that have repressive roles in active chromatin states.

Pilot Design Tiling 250 Regions at 30-Bp Resolution

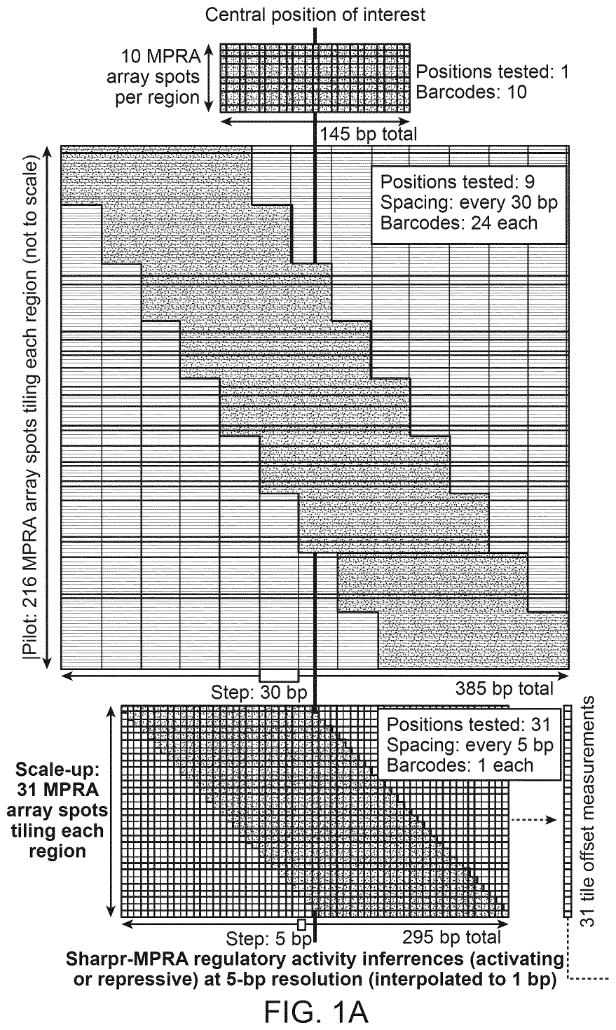

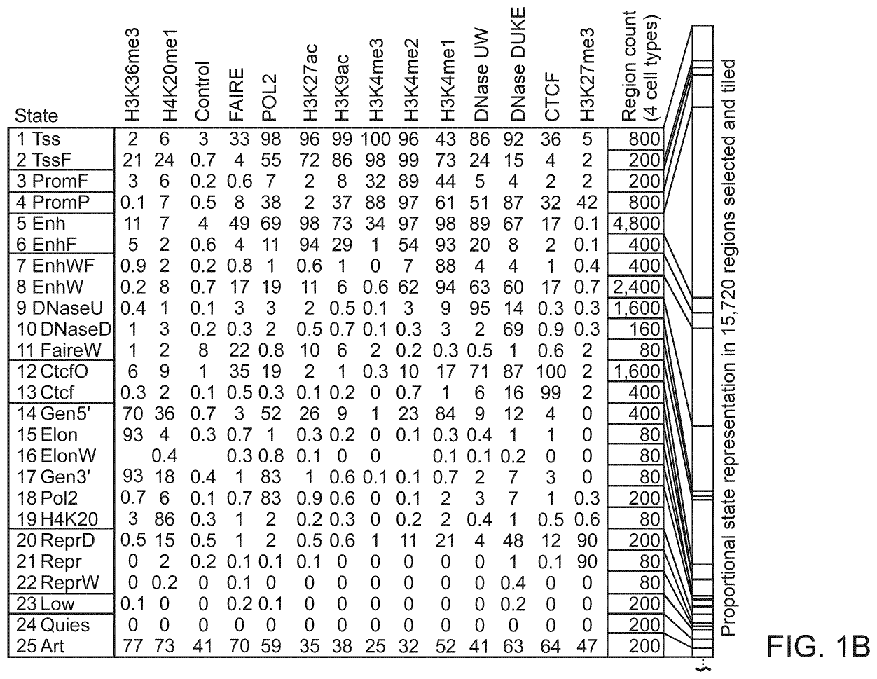

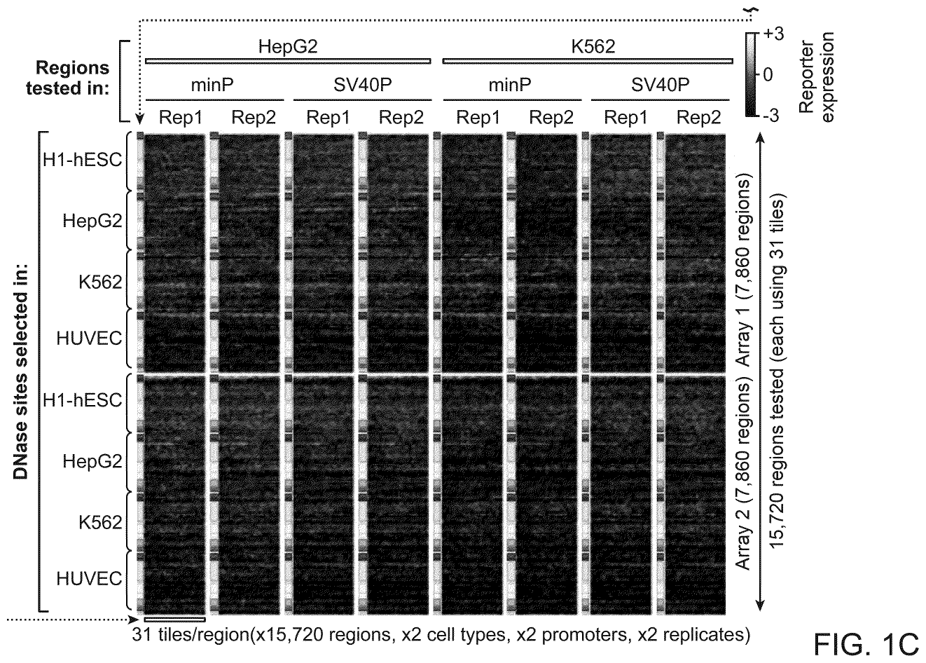

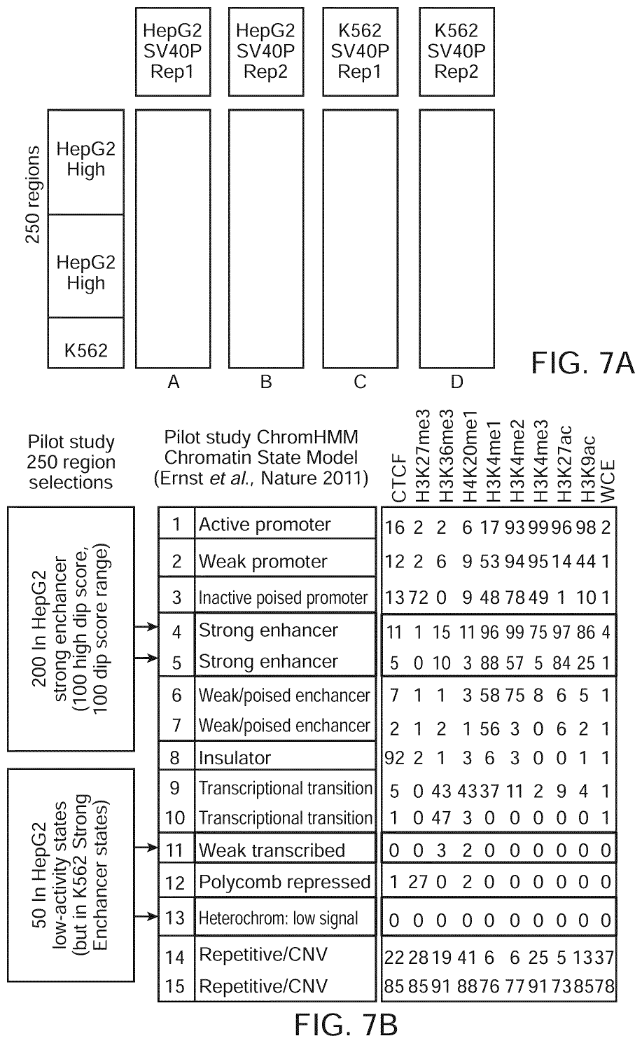

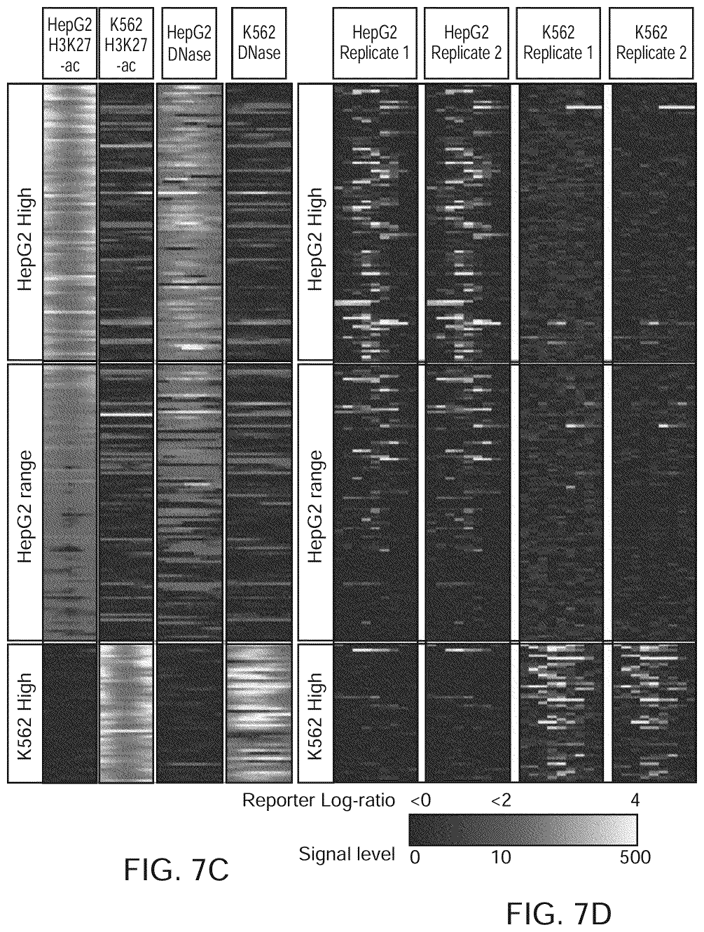

First, a low-resolution pilot design is developed and applied to 250 regions showing H3K27ac-marked enhancer chromatin states (200 in liver carcinoma HepG2 cells and 50 in leukemia K562 cells). 385-nucleotide regions at 30-nucleotide offsets are tiled using 145-nucleotide constructs, and each unique sequence is tested using 24 barcodes (FIG. 1a and FIG. 7). The tiling are centered on H3K27ac signal dips understood to be indicative of nucleosome displacement owing to transcription factor (TF) binding, and thus likely to overlap regulatory nucleotides. For HepG2 cells, 100 regions with strong dip scores were selected, and 100 regions with wide-ranging dip scores were selected (FIG. 7 and Methods). Each region is profiled in both K562 and HepG2 cells, each in two replicates.

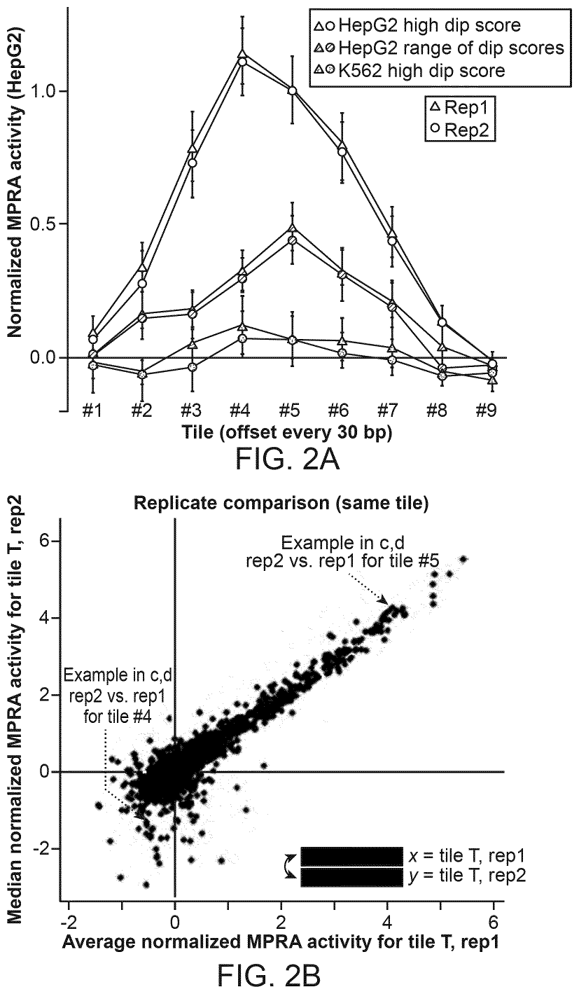

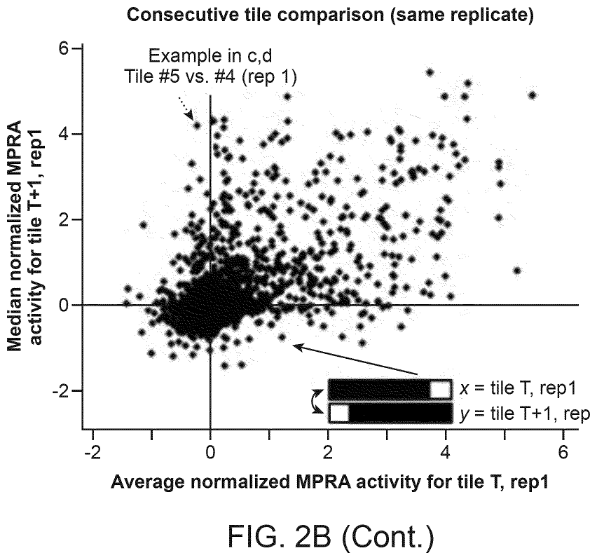

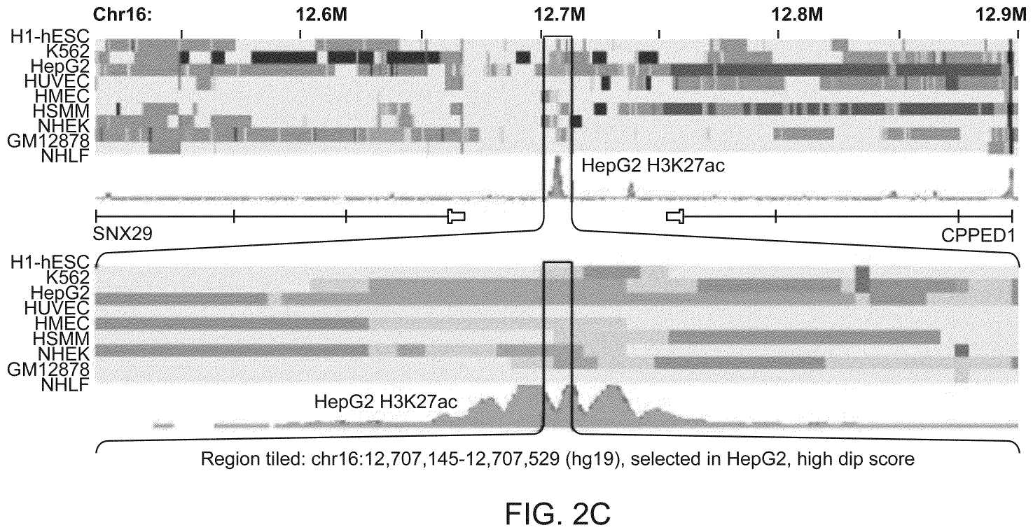

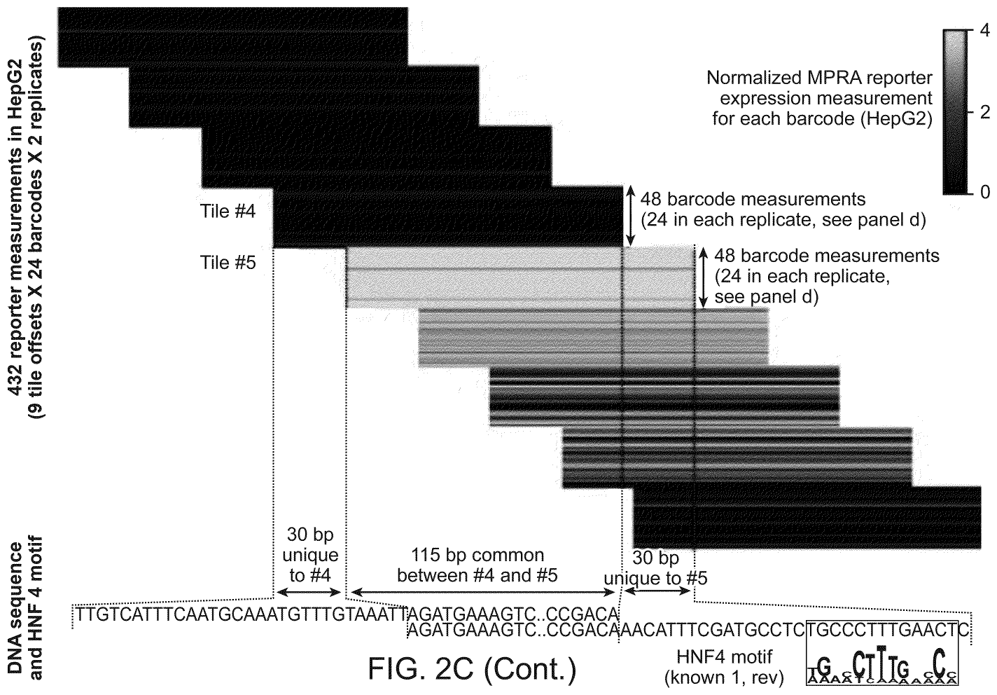

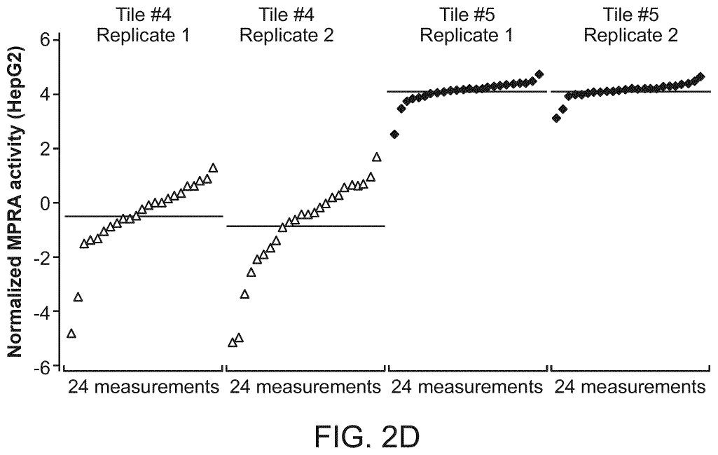

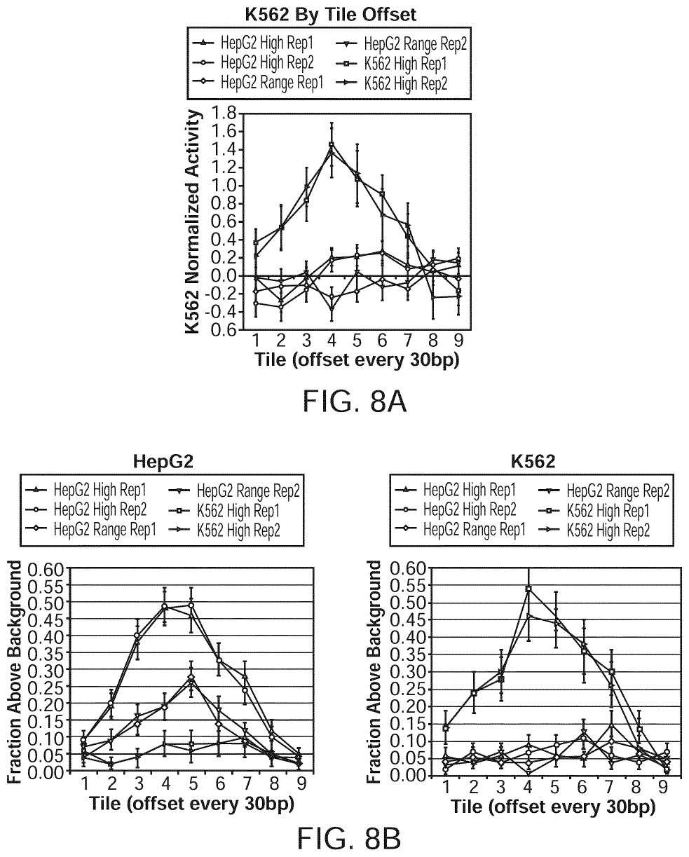

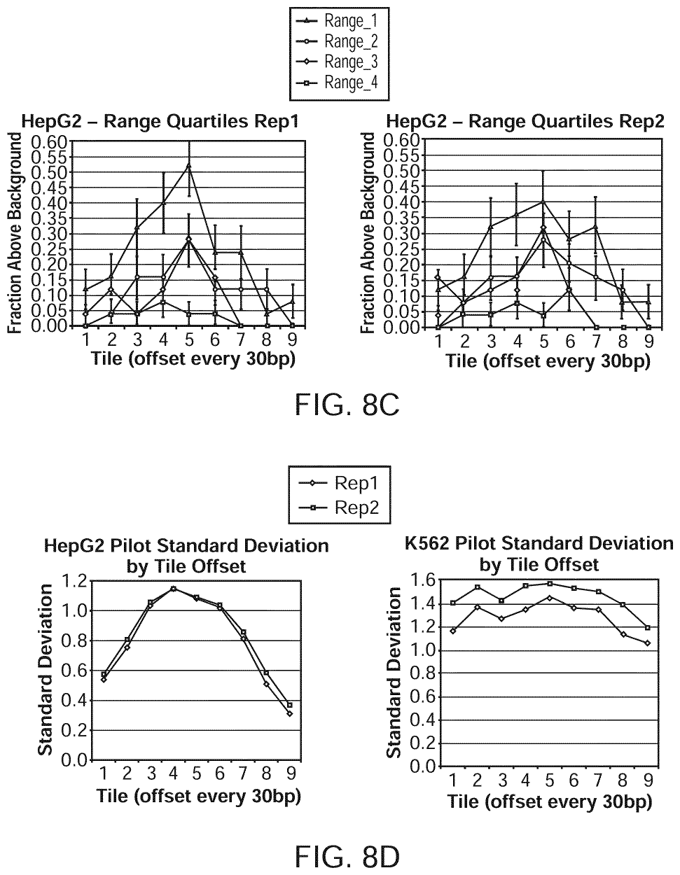

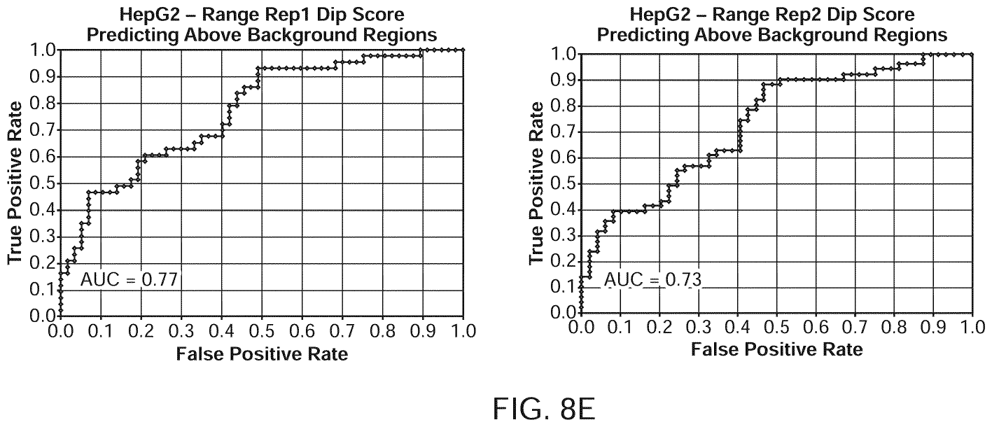

Among the nine tile positions, inner tiles (centered on H3K27ac dips) showed the highest level and frequency of activity (FIG. 2a and FIG. 8a-c), and the highest variability across regions (FIG. 8d), indicative of regulatory nucleotides. Regions with stronger H3K27ac dip scores showed higher activity, and the cell-type specificity of epigenomic signals matched the cell-type specificity of reporter expression (FIG. 2a and FIGS. 7 and 8), indicating that endogenous epigenomic information is indicative of reporter assay activity.

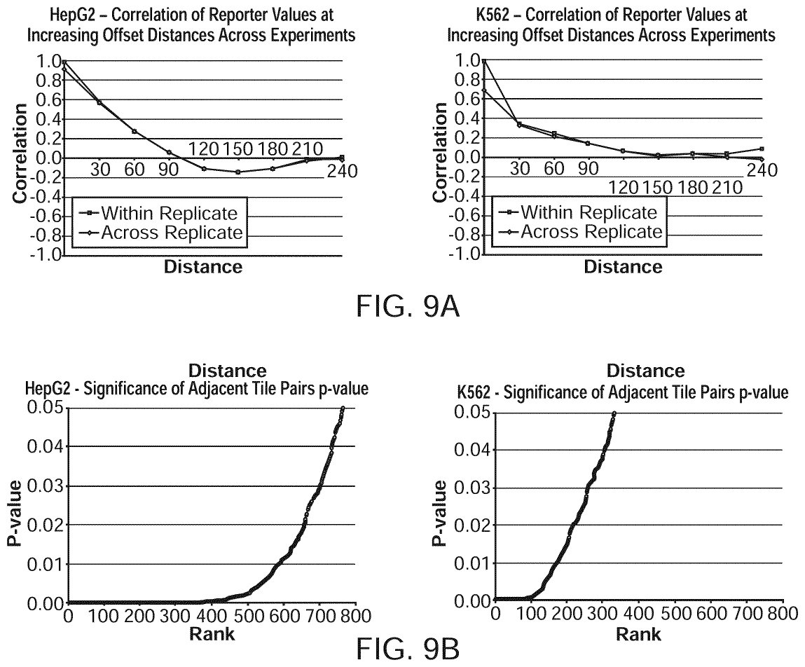

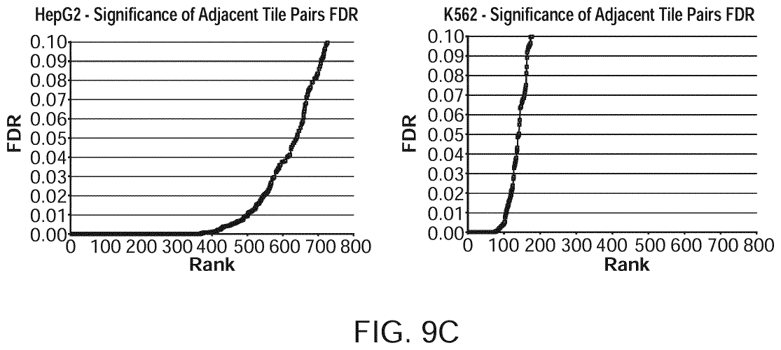

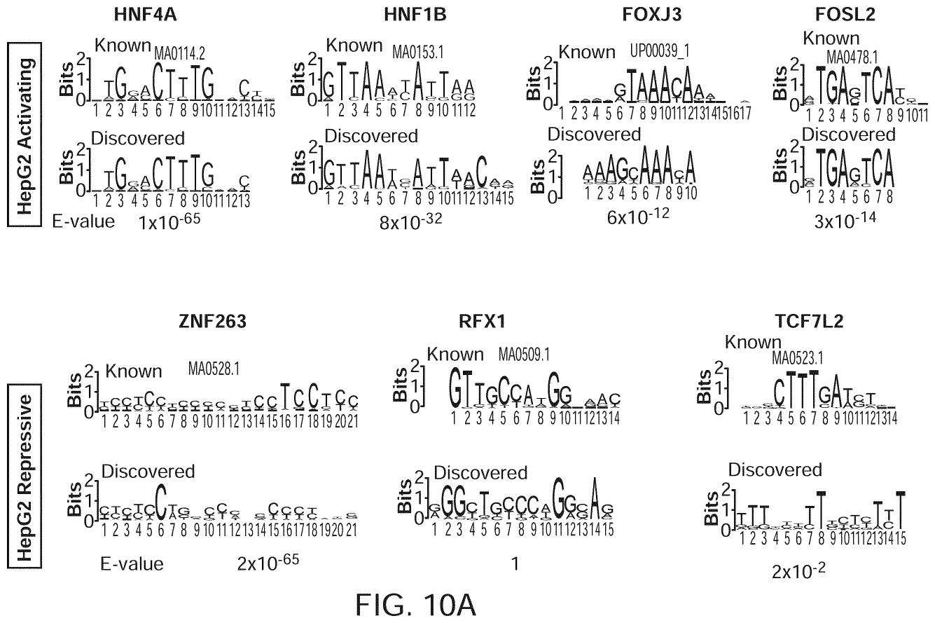

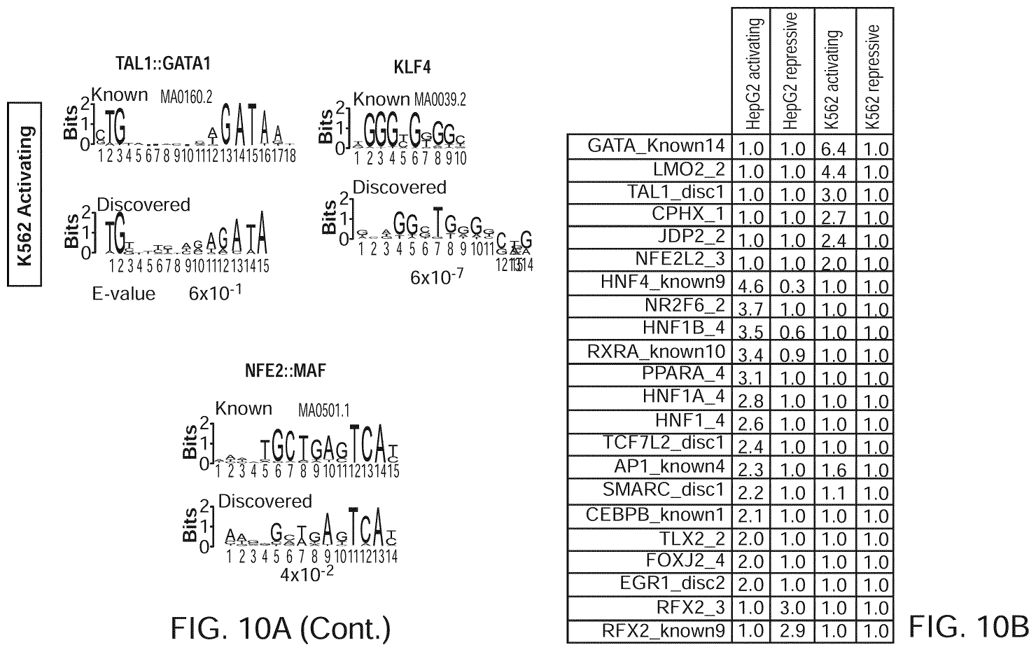

Biological replicates of the same tile showed reproducible median reporter activity (Pearson's correlation coefficient of about 0.92 across replicates for HepG2 cells), but reporter activity for tiles offset by 30 base pair (bp) sometimes differed substantially (Pearson's correlation=about 0.57 in the same HepG2 cell experiment; FIG. 2b), with tiles separated by greater distances showing greater differences in reporter activity (FIG. 9a). K562 cells showed similar results (Pearson's correlation of about 0.66 vs. about 0.34), with the lower correlation likely reflecting reduced transfection rates for K562 cells using the experimental protocols. To gain insights into the sequences driving these differences, analysis focused on 637 pairs of neighboring positions in HepG2 cells and 142 pairs in K562 cells that showed significant differences in activity (false discovery rate, about 5%; FIG. 9b,c), and a search was performed for motif differences in sequence segments distinguishing consecutive tiles (FIG. 2c,d, FIG. 10a and Methods). Segments with increased HepG2 cell activity were enriched for liver-function motifs, including HNF4 and HNF1, whereas segments with increased K562 cell activity were enriched for hematopoietic motifs, including GATA (FIG. 10b). This confirmed that a tiling approach can reveal nucleotides important for cell-type-specific regulatory function.

Scale-Up Design Tiling 15,720 Regions at 5-Bp Resolution

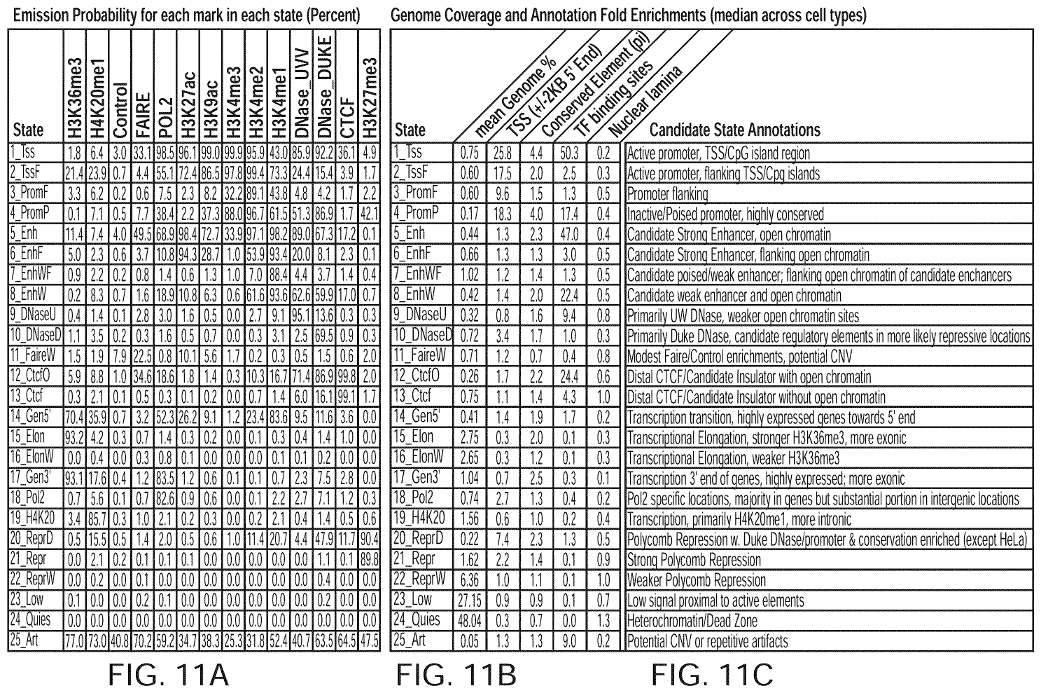

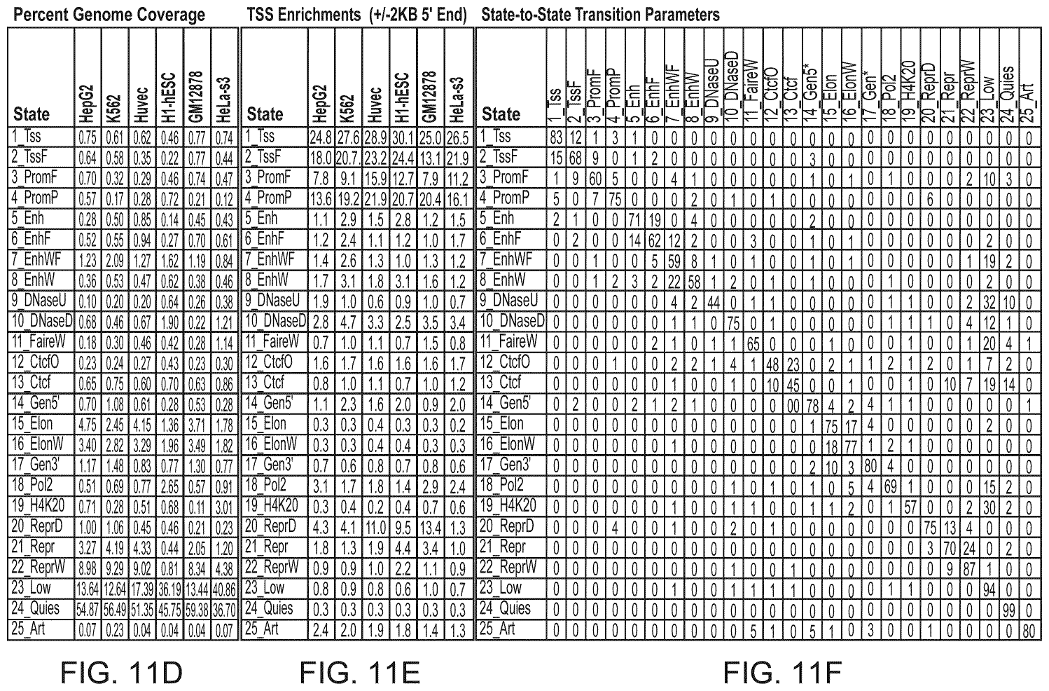

Next, the MPRA tiling design is scaled up, increasing resolution, throughput, coverage and chromatin-state diversity. To achieve these goals, several modifications were made. (i) To achieve increased resolution, reporter sequences at 5-bp increments are used instead of 30 bp (FIG. 1a). (ii) To increase throughput, a single reporter construct per position instead of 24 (FIG. 1a) is used, achieving robustness by exploiting the multiple reporter constructs overlapping most positions (15 on average; 25 for the central 105 nucleotides). Also, smaller 295-bp regions instead of 385-bp regions are tested, focusing on the most informative positions based on the pilot results (FIG. 2a and FIG. 8). (iii) To increase coverage, two 244K arrays for DNA synthesis (instead of a single 55K array) are used, targeting 15,720 regions for tiling, each profiled in both HepG2 and K562 cells, using both a minimal TATA promoter (minP) and a strong SV40 promoter (SV40P), each in two replicates (FIG. 1c), resulting in a total of 3.9 million measurements. (iv) To allow analyses across diverse chromatin states of a 25-state ChromHMM model (FIG. 11), regions on double-cut DNase I hypersensitive sites are centered instead of H3K27ac dips. To ensure representation of all states, a tiered random sampling approach is used, which also favored enhancers and other DNase-hypersensitivity-enriched states (FIG. 1b). To include both active and inactive regulatory sites in the cell types profiled, DNase sites from HepG2 and K562 cells are selected, as well as two additional cell types: human umbilical vein endothelial cells (HUVECs) and human embryonic stem cells (H1-hESC) (FIG. 1c).

These design choices, and the SHARPR computational inference method described herein next, allowed the inference of regulatory activity at about 5-nucleotide resolution across about 4.6 million nucleotides spanning over about 15,000 regions, an about six fold increase in resolution and an about 60-fold increase in coverage compared to the pilot design.

Inference of Activating and Repressive Nucleotides

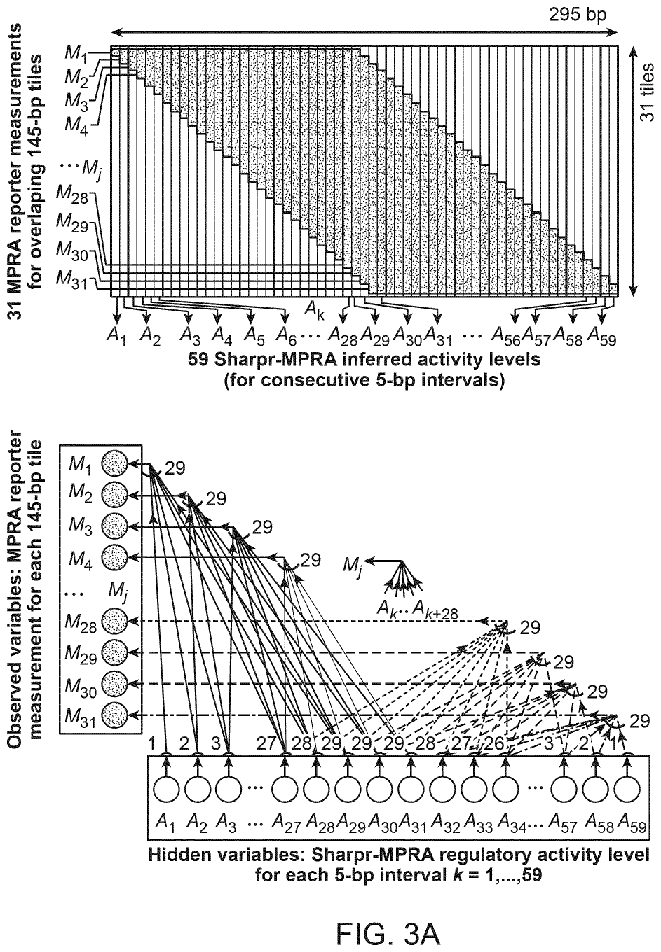

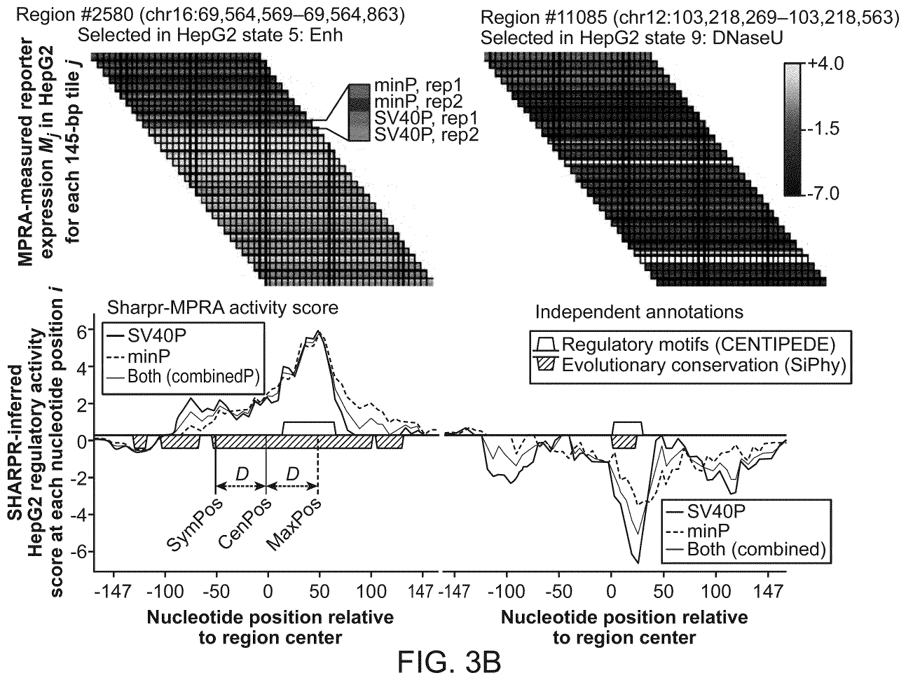

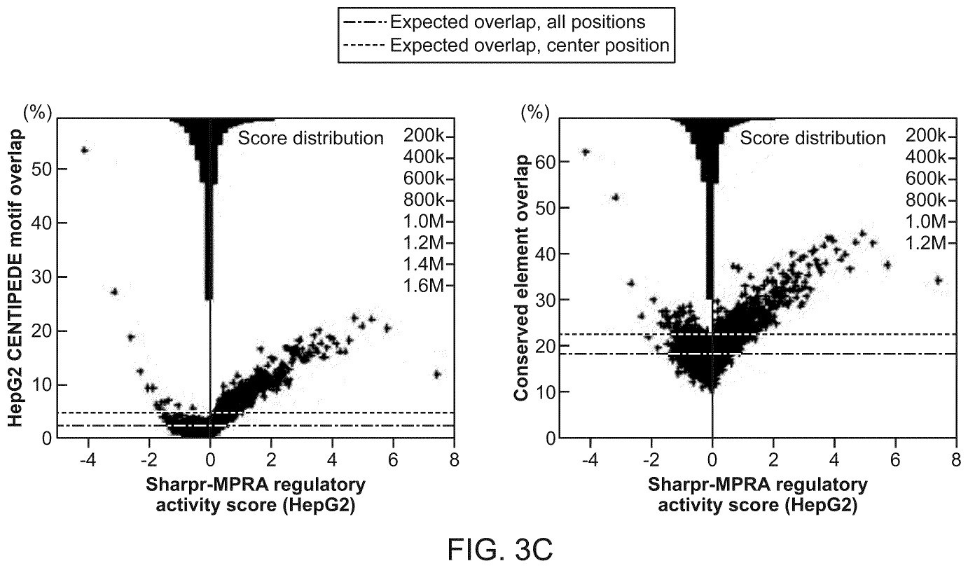

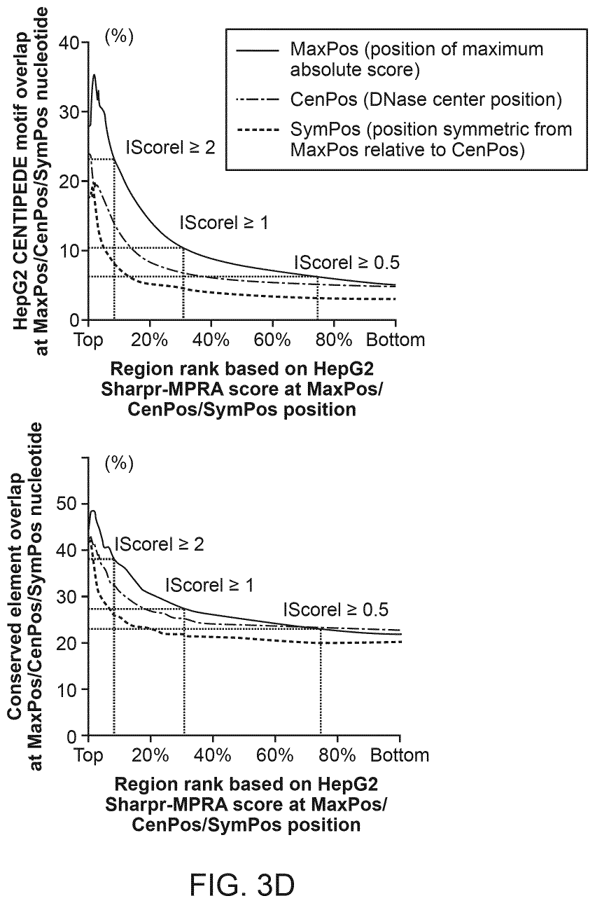

A computational method, SHARPR, is developed, to score the relative activating or repressive potential of each 5-bp interval in tiled regions and to interpolate these values to yield predictions for individual nucleotides (FIG. 3a, b). The inclusion and exclusion of 5-bp nucleotide intervals between consecutive allow inferences at substantially higher resolution (5 bp) than with other reporter constructs (145 bp) (FIG. 3a). It is reasoned that activating intervals (for example, containing activator motifs) should increase the reporter expression for tiles overlapping them, whereas repressive intervals (for example, containing repressor motifs) should decrease reporter expression of overlapping tiles, as it is shown in the pilot experiments (for example, FIG. 2c). Thus, modeling the relative activity of overlapping tiles should allow inference of activating and repressive nucleotide positions at high resolution (FIG. 3b).

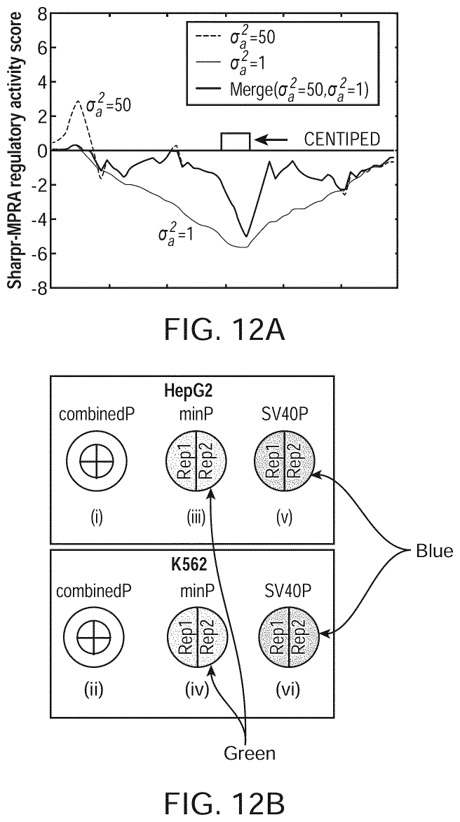

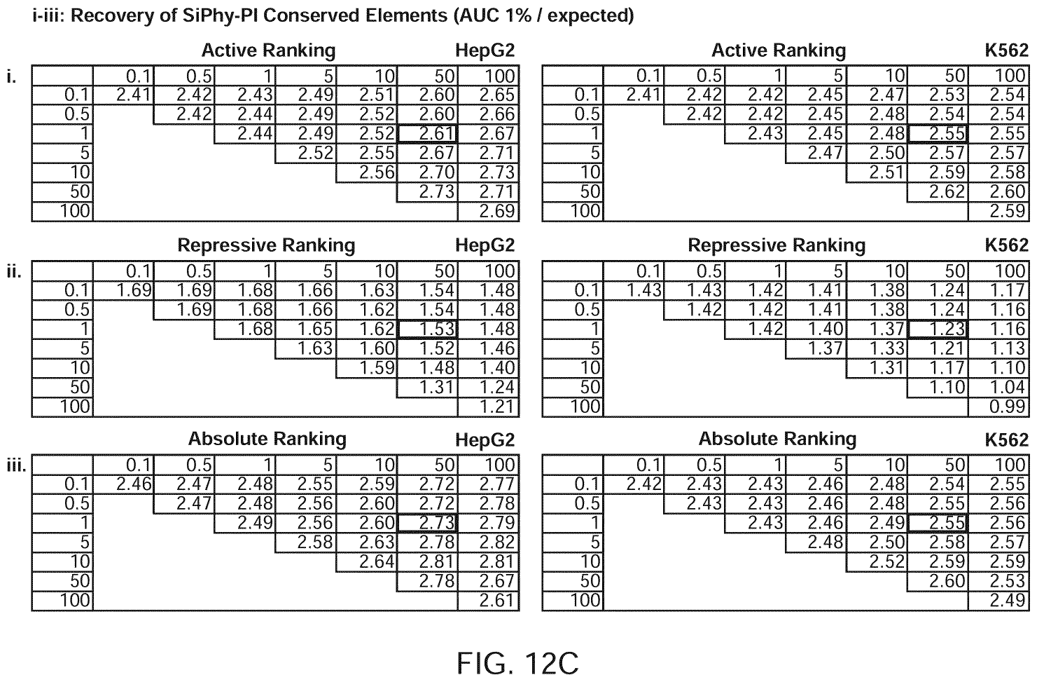

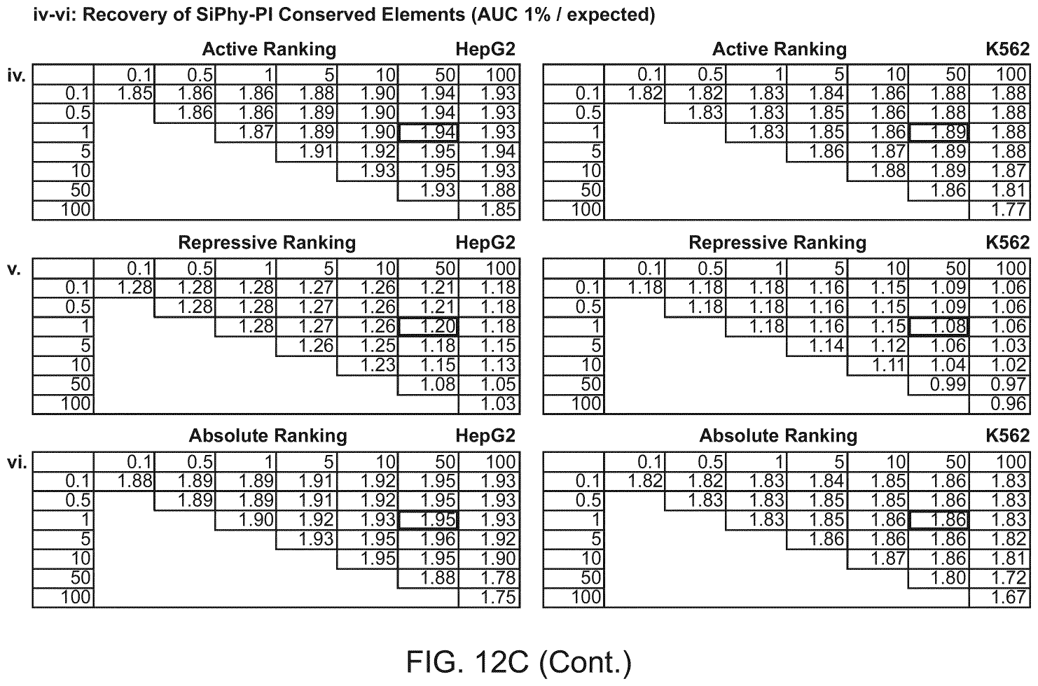

A probabilistic graphical model (FIG. 3a) is constructed to relate the unobserved regulatory activity of each 5-bp interval (hidden variables A₁-A₅₉) to the 145-bp reporter measurements (observed variables M₁-M₃₁). M is modeled using a normal distribution with a mean corresponding to the average of overlapping Aₖ and a variance corresponding to the empirical variance of all measurements in the experiment (Methods and Supplementary Note 1). Aₖ is modeled using a normal distribution with a mean calculated as the average of all measurements in the experiment and a variance σ²_{α} as a free parameter (smaller values resulting in more smoothed inferences). A low-variance prior and a high-variance prior are used, and results are combined (Methods and Supplementary Note 2). The “most likely” values for the regulatory activity variables are inferred based on observed reporter measurements and their prior distributions, and the values are standardized using a z score to combine results from multiple replicates, promoters and variance settings (FIG. 12). Piecewise linear interpolation from the 5-bp activity estimates was carried out to infer the regulatory activity of each nucleotide in the tiled regions (Methods and Supplementary Note 3). The computational portion of Sharpr-MPRA was implemented in a software package.

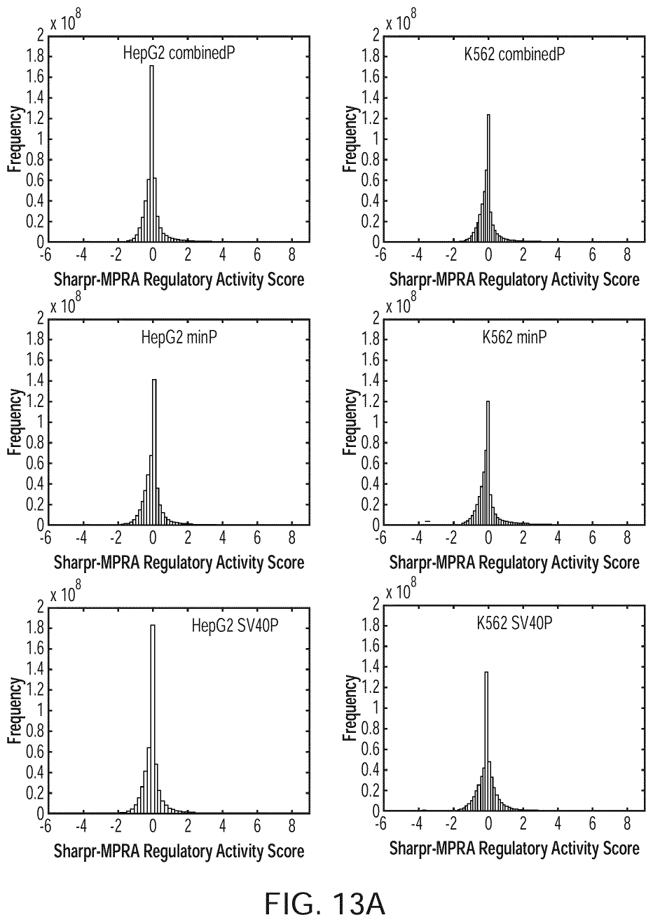

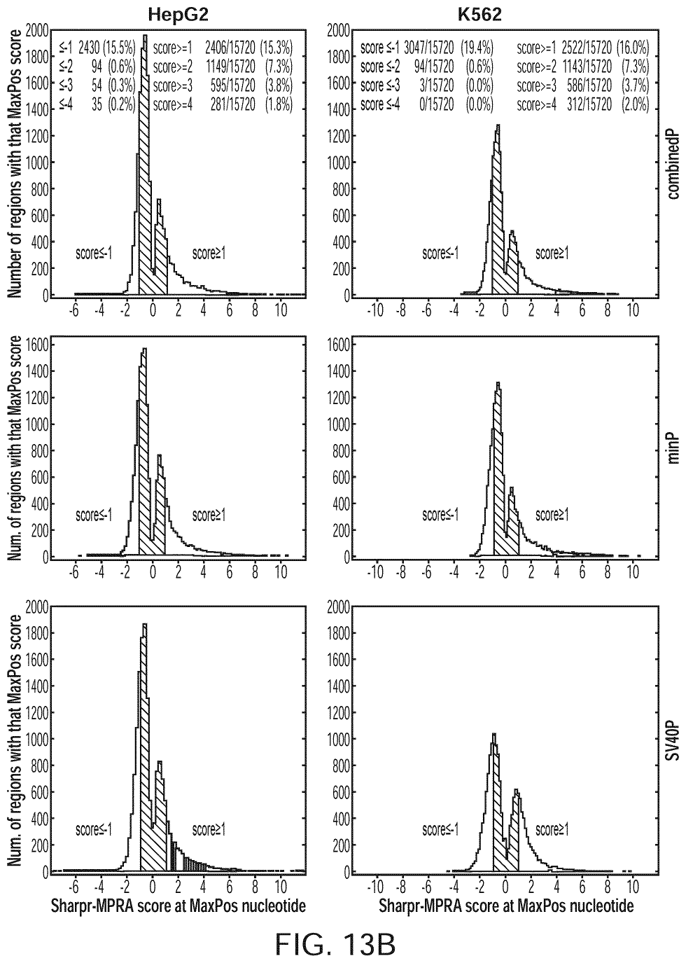

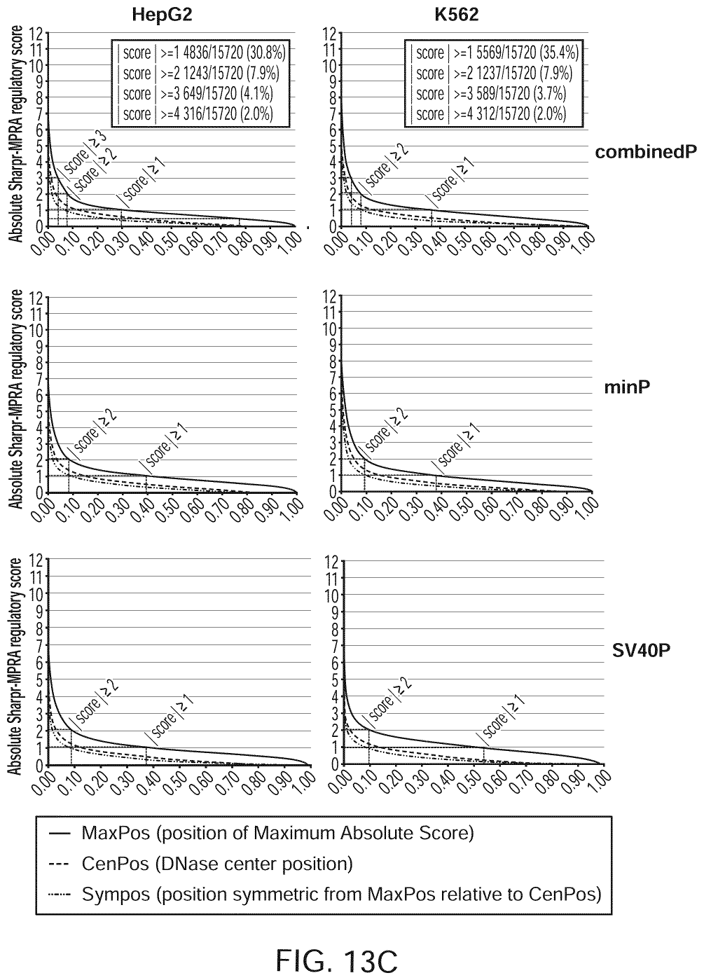

Sharpr-MPRA is used to make activating or repressive regulatory activity inferences for about 4.6 million nucleotides, each in two cell types, each using two promoter types, each using two replicates. Inferred nucleotide activity are made for both minP and SV40P individually (combining two replicate experiments for each promoter), and for their combination (combinedP, using four experiments jointly), resulting in three activity tracks for each cell type (Supplementary FIGS. 12b and 13a). Also, minP, SV40P and combinedP scores are assigned to each region (Supplementary FIG. 13b,c), using the signed (activating or repressive) score of the maximum absolute score position (MaxPos) (FIG. 3b). Visualizations are provided showing all minP, SV40P and combinedP inferences for HepG2 and K562 cells.

Reproducibility of Sharpr-MPRA Regulatory Activity Scores

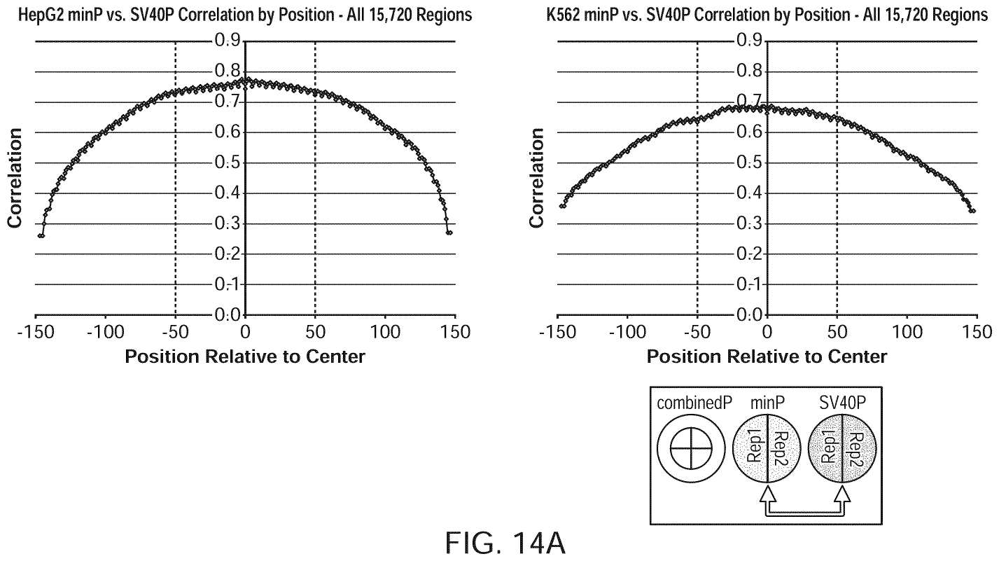

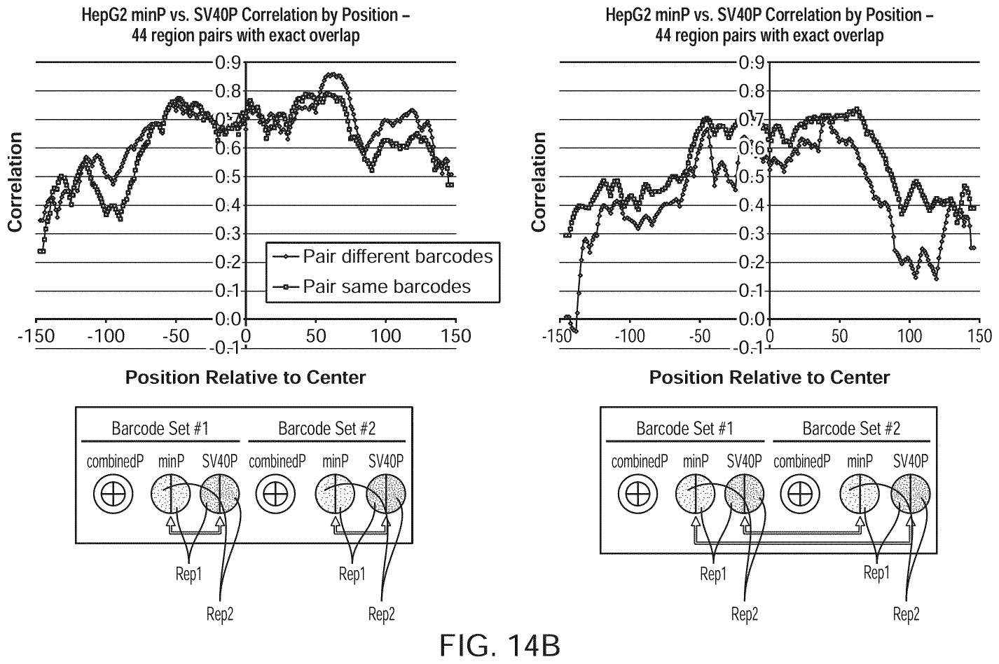

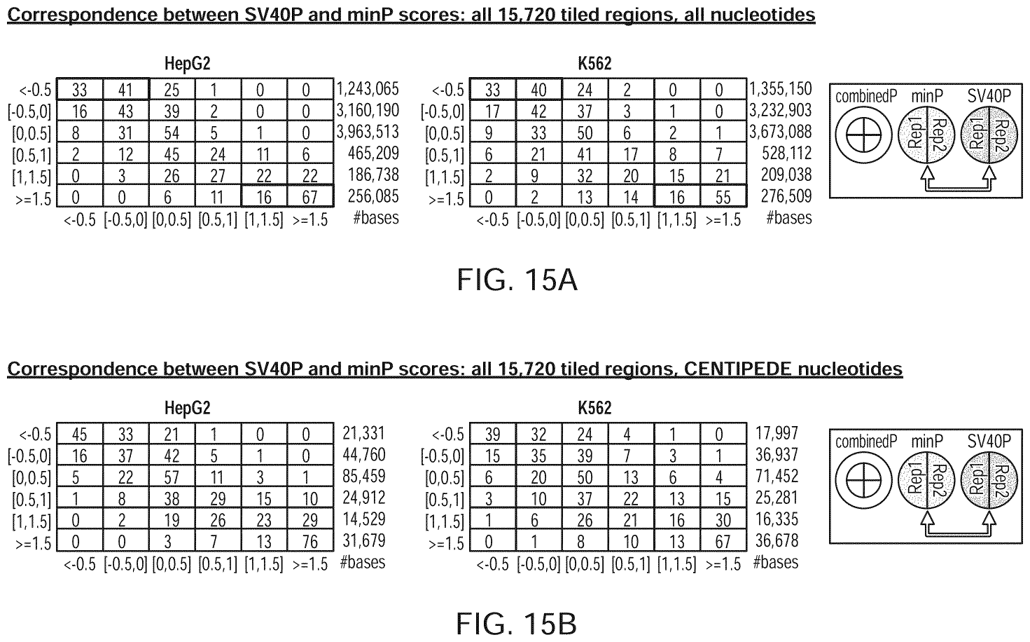

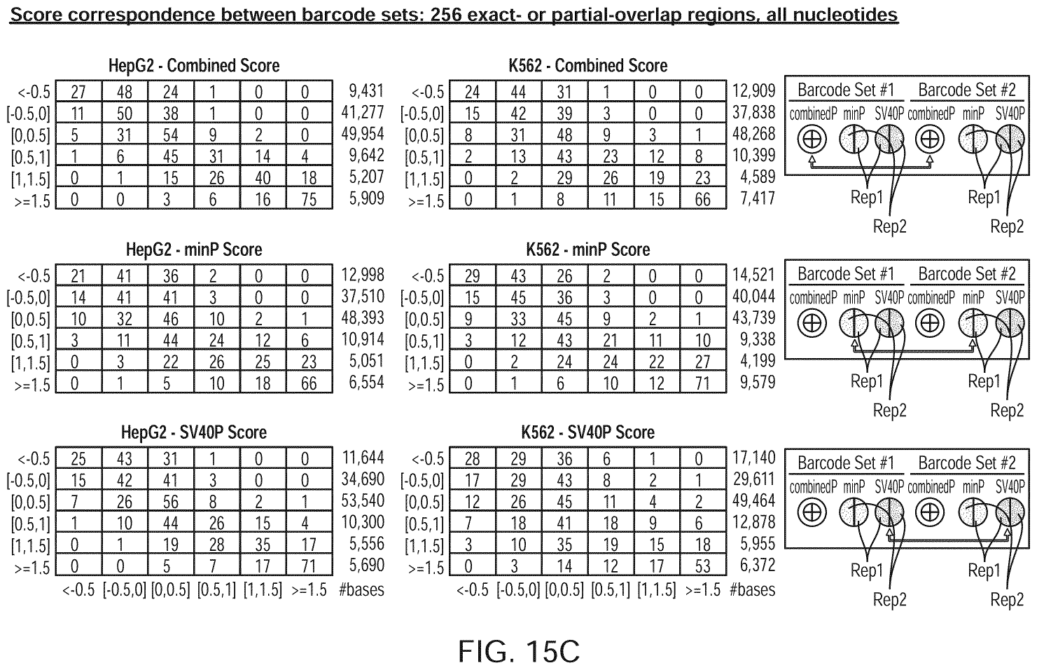

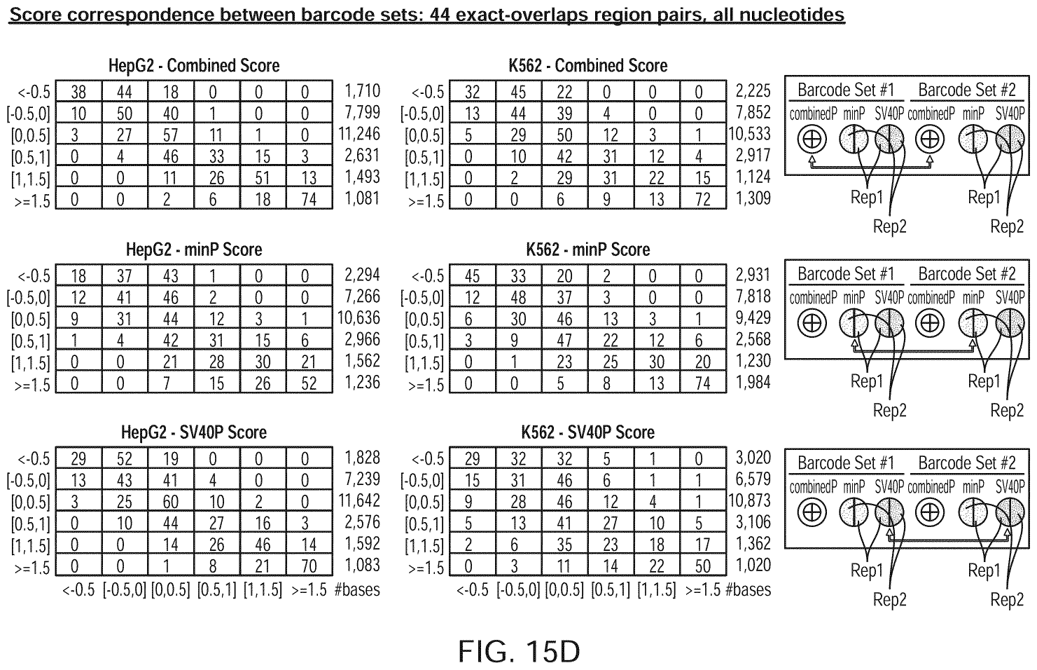

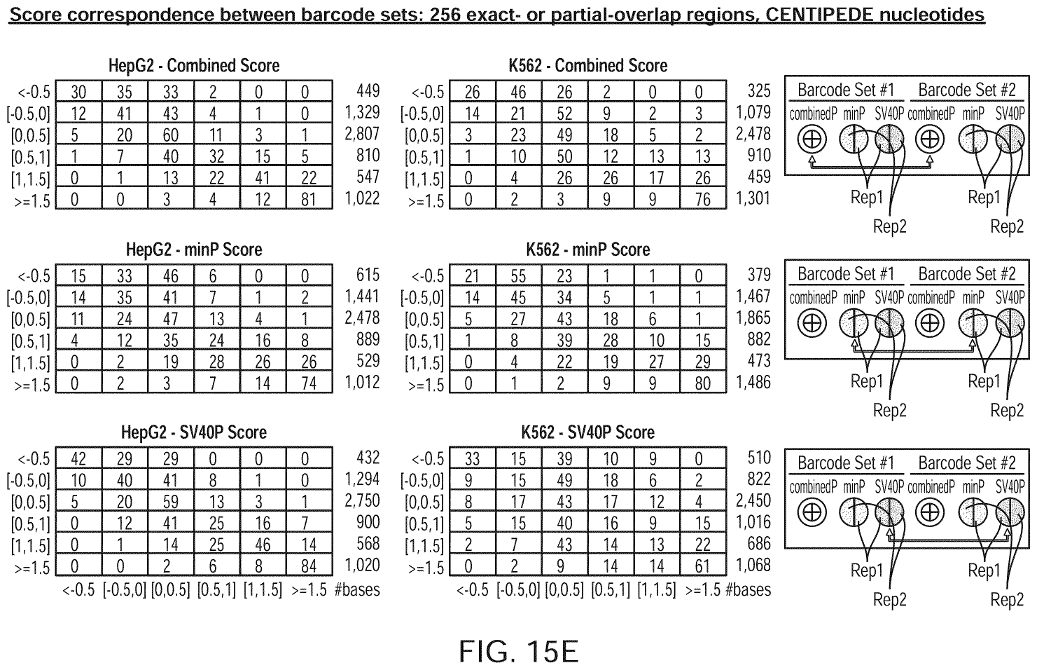

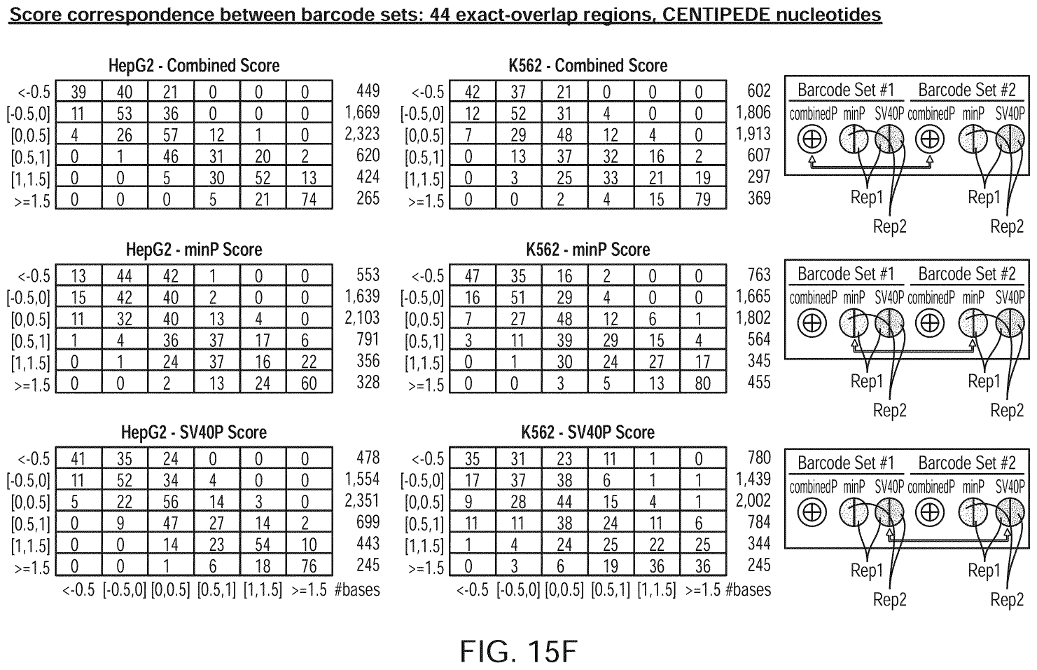

The reproducibility of Sharpr-MPRA scores are evaluated in multiple ways. First, the agreement between minP and SV40P inferences for each nucleotide position across regions is evaluated. The central 101 positions showed on average about 0.75 Pearson's correlation coefficient for HepG2 cells and about 0.66 for K562 cells, which decreased toward outer positions (FIG. 14a), attributable to fewer tiles overlapping outer positions and stronger regulatory activity closer to DNase site centers. Individual nucleotides maintained similar scores between promoter types (FIG. 15a), with about 83% of scores ≥1.5 for one promoter showing scores ≥1 for the other in HepG2 cells (about 71% for K562 cells), and about 74% of scores <−0.5 for one promoter showing scores <0 for the other in HepG2 cells (about 73% for K562 cells).

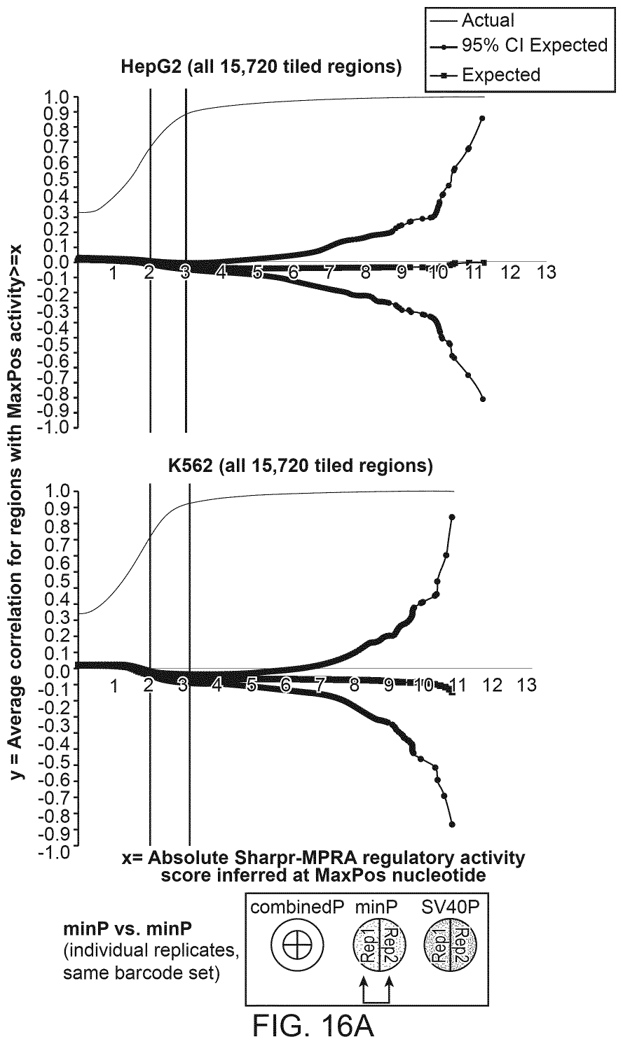

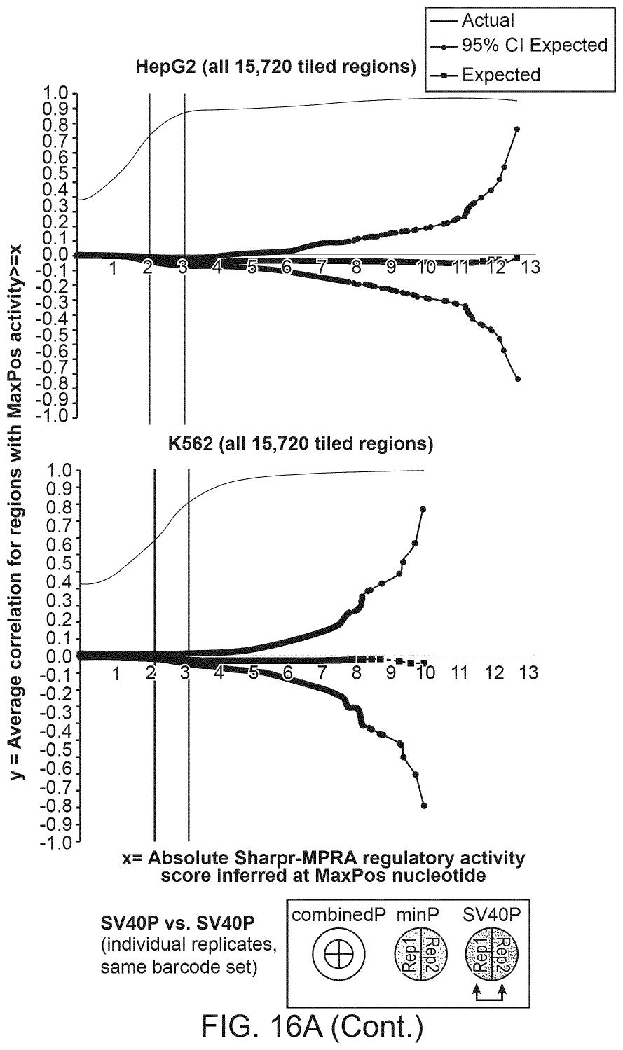

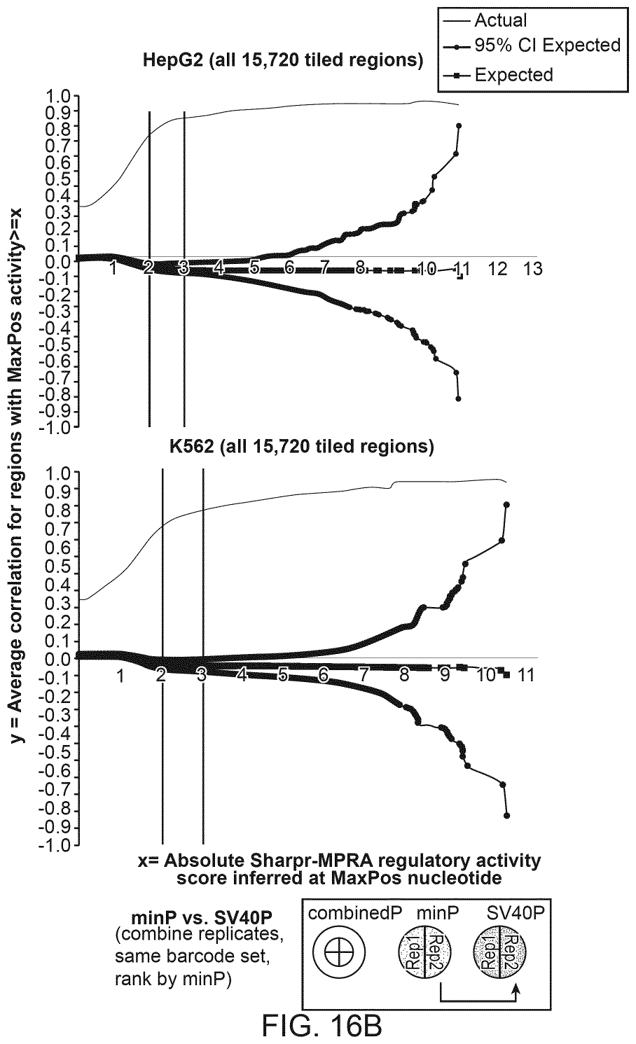

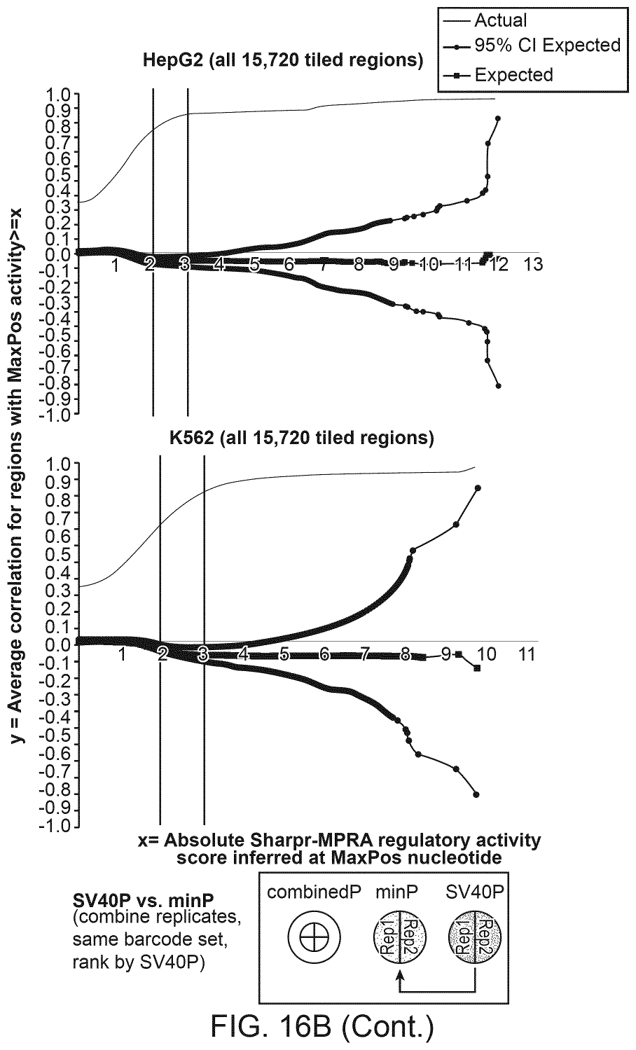

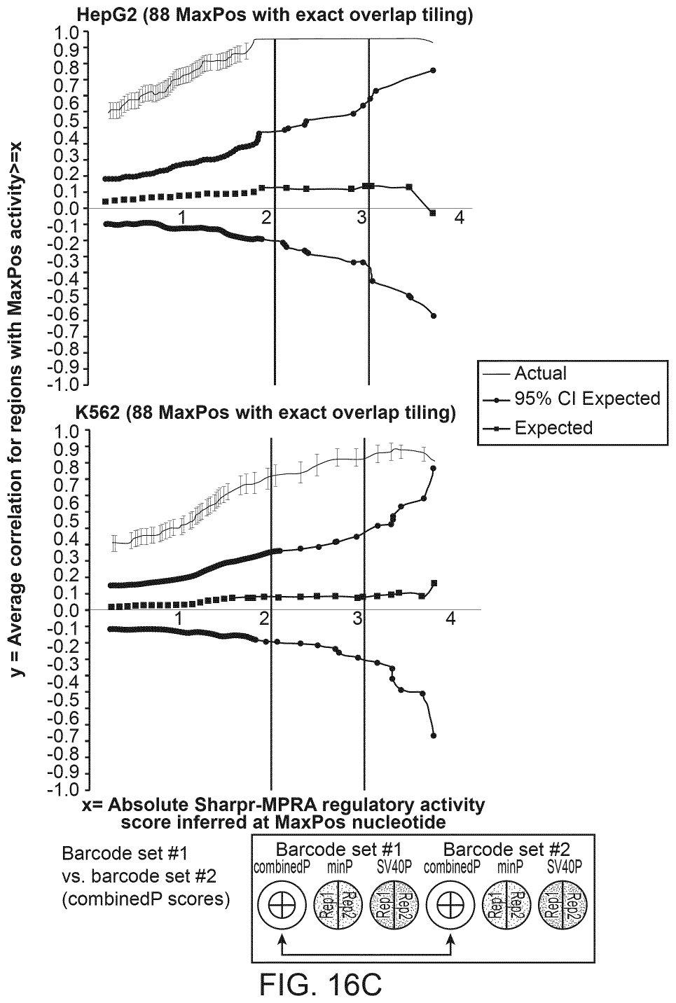

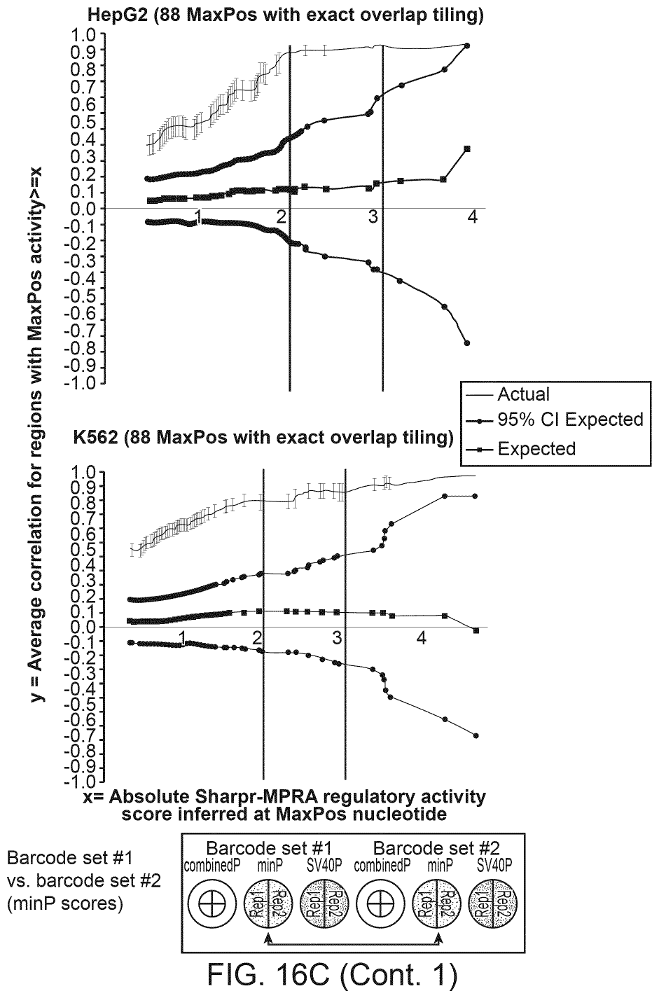

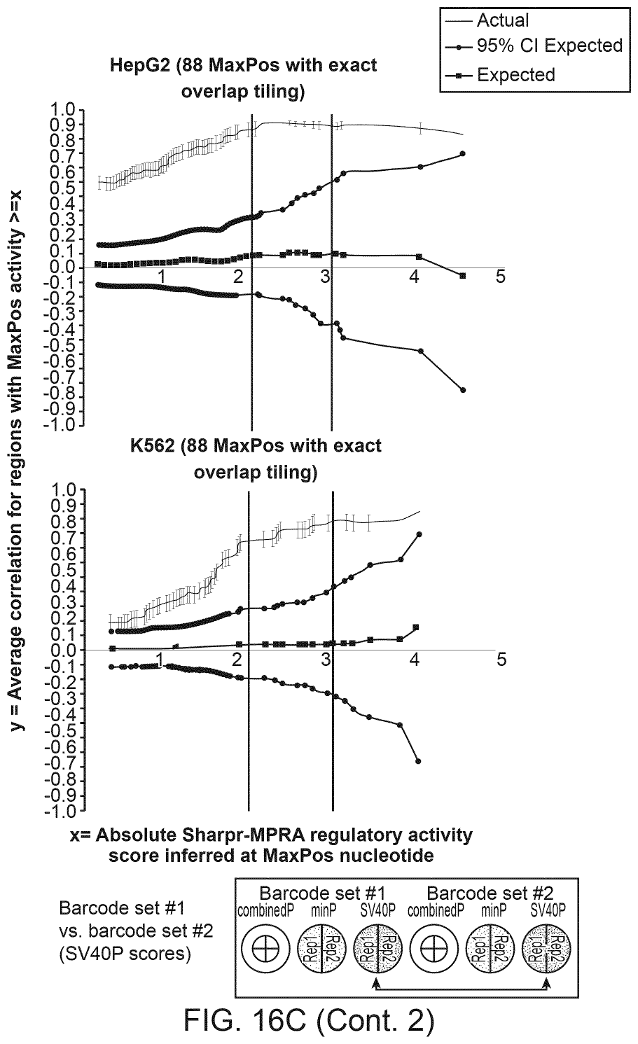

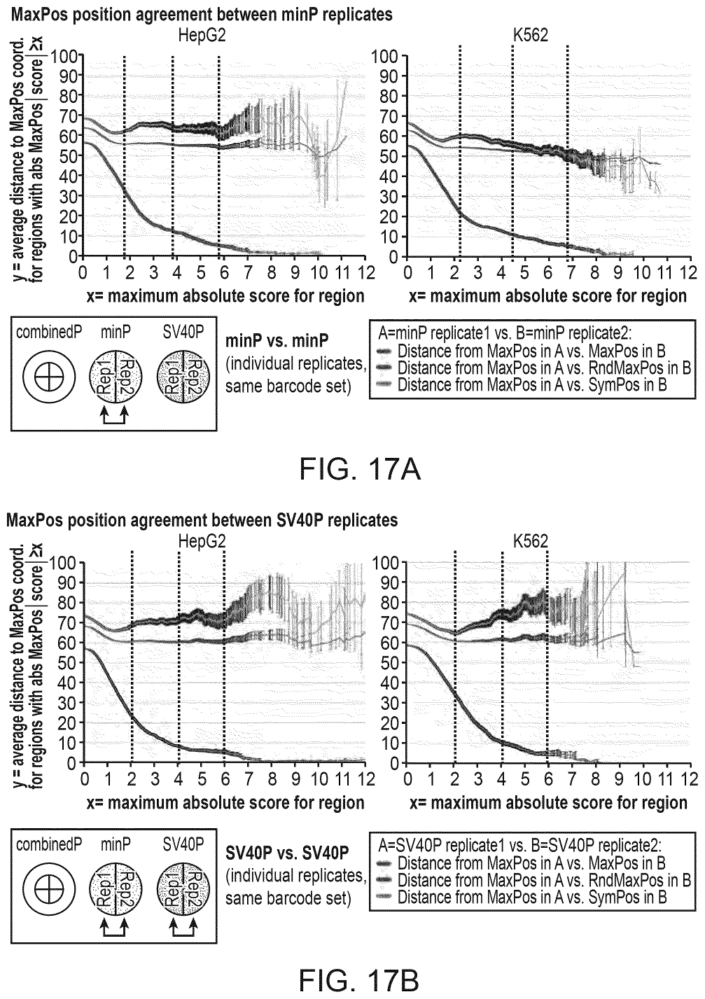

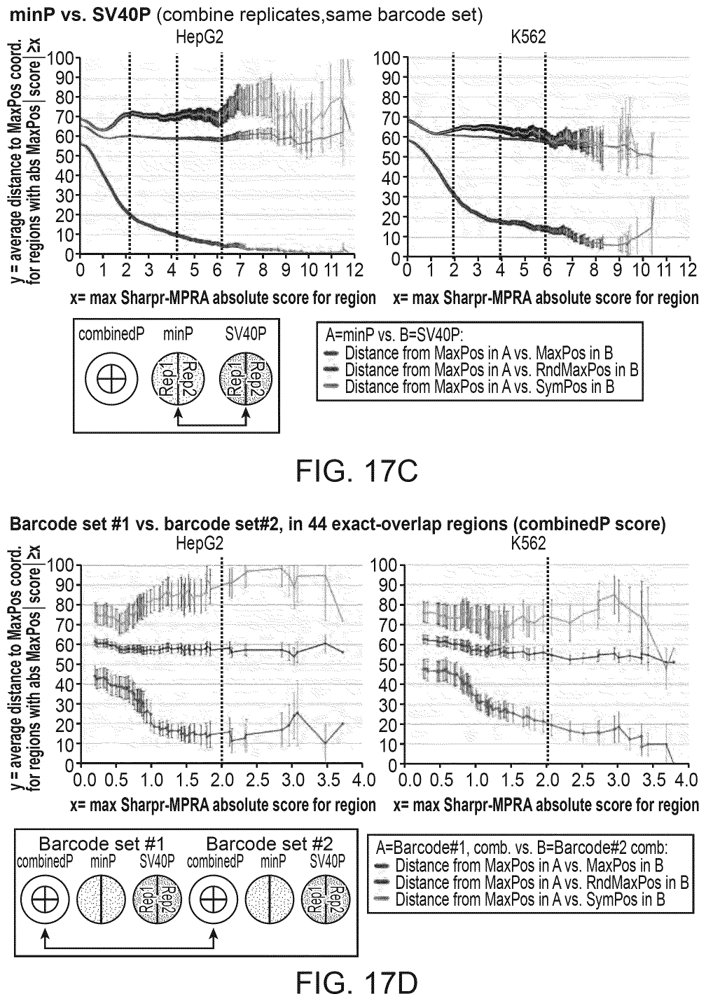

Individual replicates of each promoter type showed strong agreement for increasing absolute scores (FIG. 16a) for both cell types and both promoter types (for example, Pearson's correlation of about 0.7 on average for regions with |score|≥2, and about 0.9 for |score|≥3 for minP). Between promoter types, regions with |score|≥2 showed about 0.8 correlation on average in HepG2 cells (about 0.7 in K562 cells), compared to <0.1 expected by chance (FIG. 16b). The MaxPos nucleotide position showed substantially greater concordance between replicates and between promoter types than expected by chance (FIG. 17a-c), with the distance decreasing with increasing absolute score. Between minP replicates in HepG2 cells, MaxPos nucleotides were on average within about 28 bp, about 11 bp and about 5 bp for |score|≥2, 4 and 6, respectively, for HepG2 cells (about 26 bp, about 13 bp and about 7 bp, respectively, for K562 cells), compared to about 60 bp expected by chance when sampling MaxPos nucleotide positions.

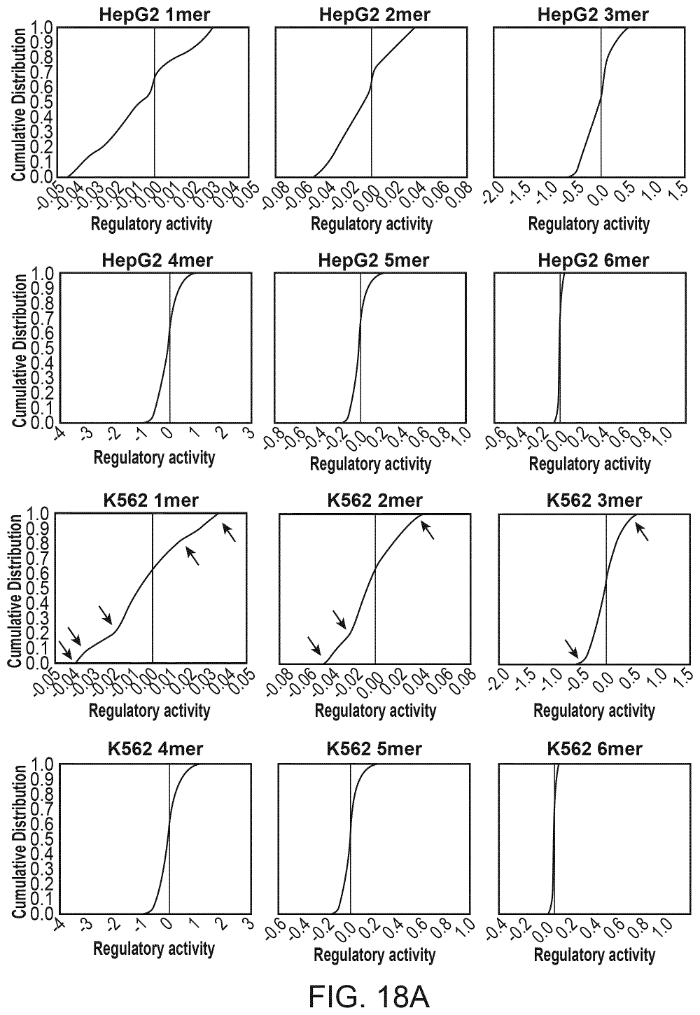

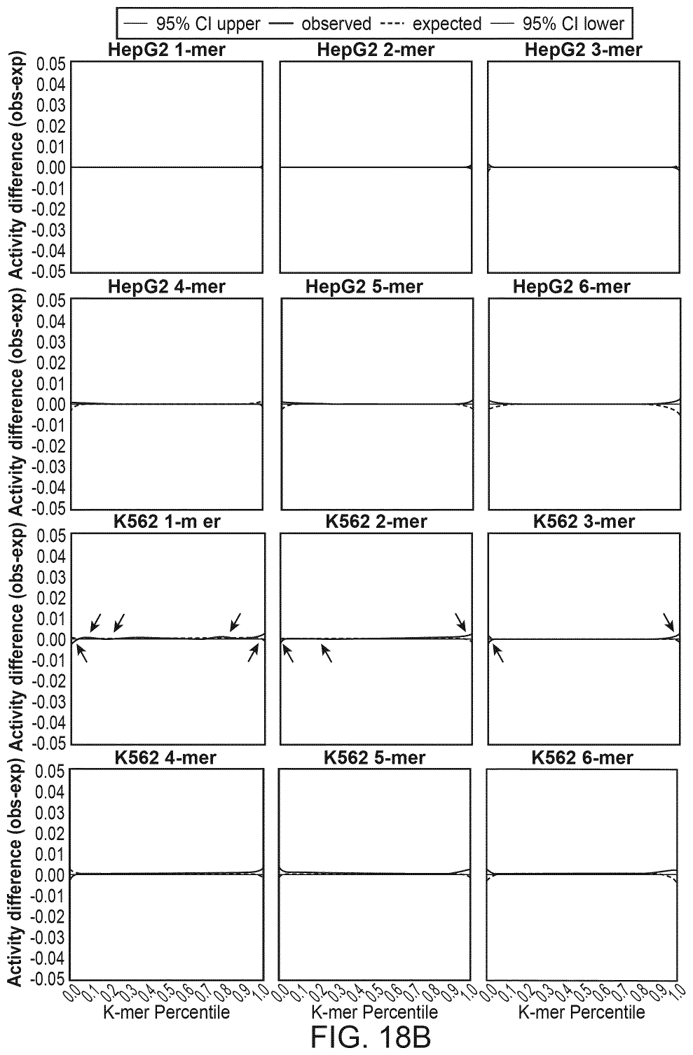

These comparisons are repeated across different sets of barcodes using DNase sites that are independently selected in multiple cell types, and thus tested using multiple barcode sets. Forty-four regions overlapped completely and 212 overlapped partially, providing a resource for quantifying potential barcode effects. The position-specific correlation in Sharpr-MPRA scores between SV40P and minP showed a negligible change in HepG2 cells and a modest reduction in K562 cells when using the same versus a different set of barcodes (about 0.72 vs. about 0.71 and about 0.69 vs. about 0.60, respectively, for the central 101 nucleotide positions on average; FIG. 14b), indicating a modest barcode effect relative to other sources of biological and technical variability. Individual nucleotide scores showed strong agreement across barcode sets for regions of both partial overlap and exact overlap (FIG. 15c,d), with about 90% of combinedP scores >1.5 in one showing combinedP score ≥1 in the other for HepG2 cells (about 80% in K562 cells) across all 256 multiply tiled regions. The 44 complete-overlap regions showed high score correlation across barcode sets for regions with high absolute scores (about 0.96 for HepG2 cells and about 0.77 for K562 cells for |score|≥2; FIG. 16c). The position of maximum score was within about 16 bp between barcode sets for HepG2 cells (about 21 bp for K562 cells) for |score|≥2 (FIG. 17d). A search for k-mer effects in the barcodes (Methods) revealed no influence on inferred activity in HepG2 cells compared to random expectation, a small effect for shorter k-mers in K562 cells, and no noticeable effect for longer k-mers in either cell type (FIG. 18).

Sharpr-MPRA Recovers Motifs and Conserved Regions

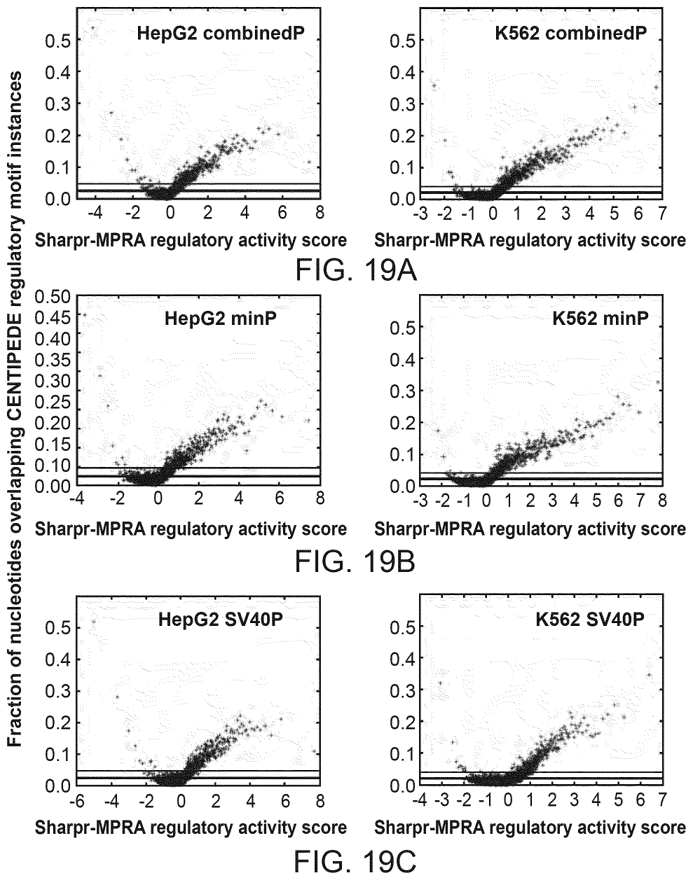

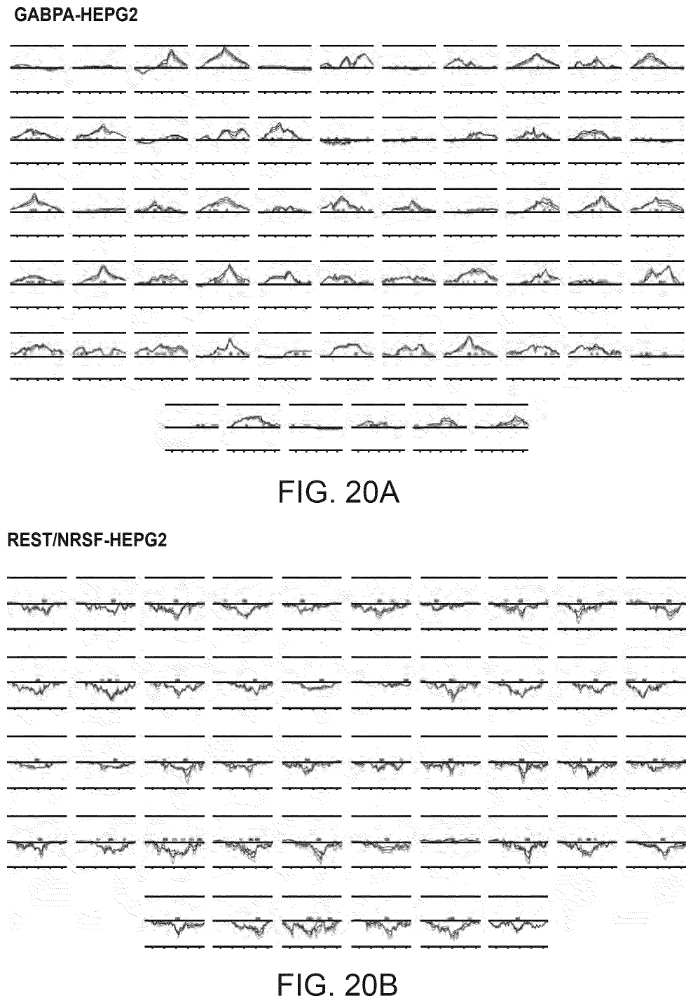

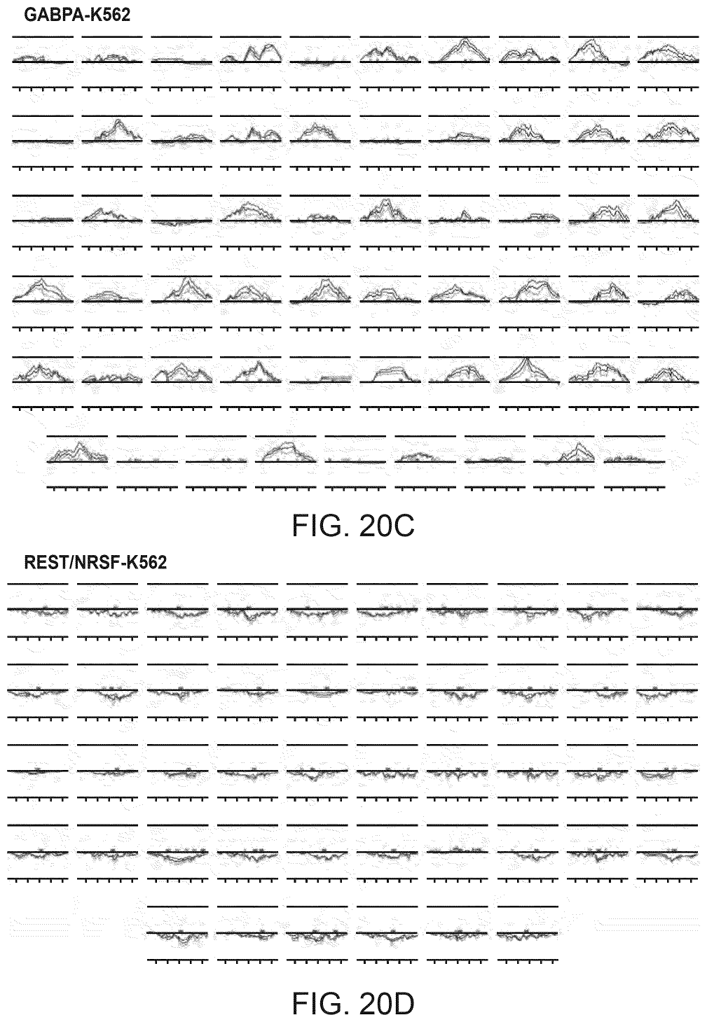

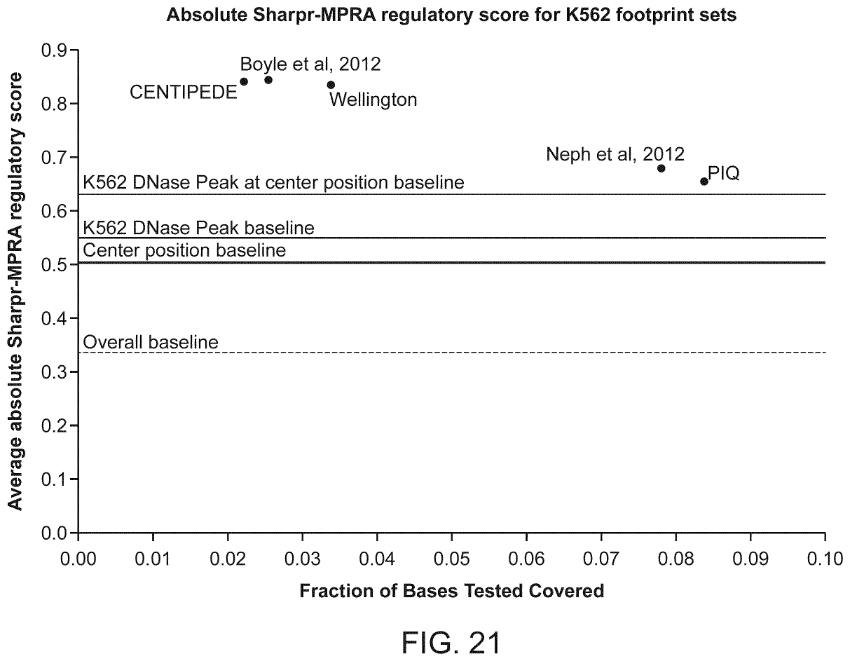

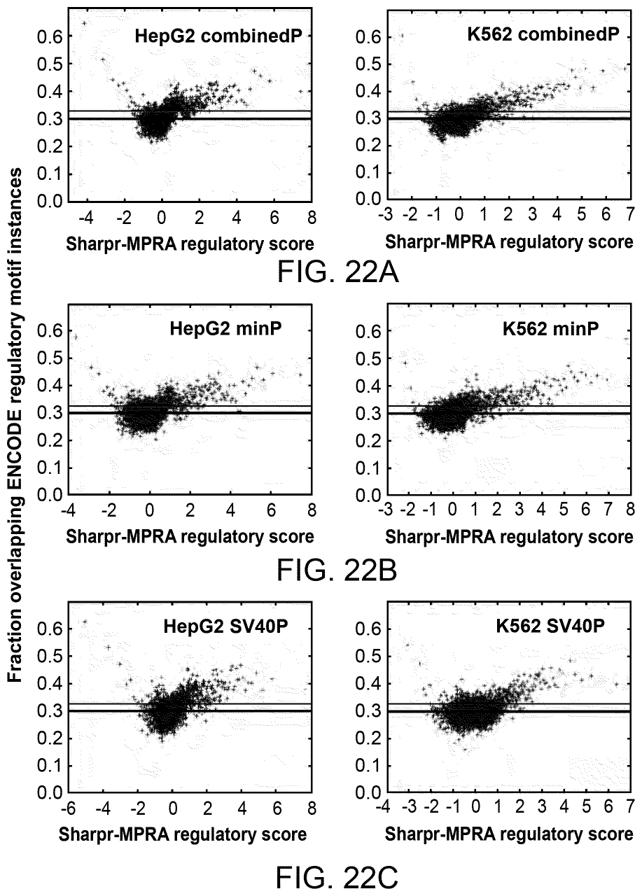

To establish whether the inferred regulatory nucleotides were biologically relevant, they are compared to predictions of transcription factor (TF) binding sites that are not used to make the inferences, including DNase-based and DNase-independent predictions of TF-bound nucleotides, and both motif-based and motif-independent predictions of regulatory nucleotides. For example, CENTIPEDE motif annotations showed strong agreement for both activating and repressive scores at the nucleotide level (FIG. 3c and FIG. 19), at the region level (FIG. 3b) and for specific regulators (FIG. 20), and CENTIPEDE nucleotides showed reproducible scores (FIG. 15b,e,f). All five regulatory annotation sets tested showed better agreement with the inferences than with stringent controls (FIGS. 21 and 22).

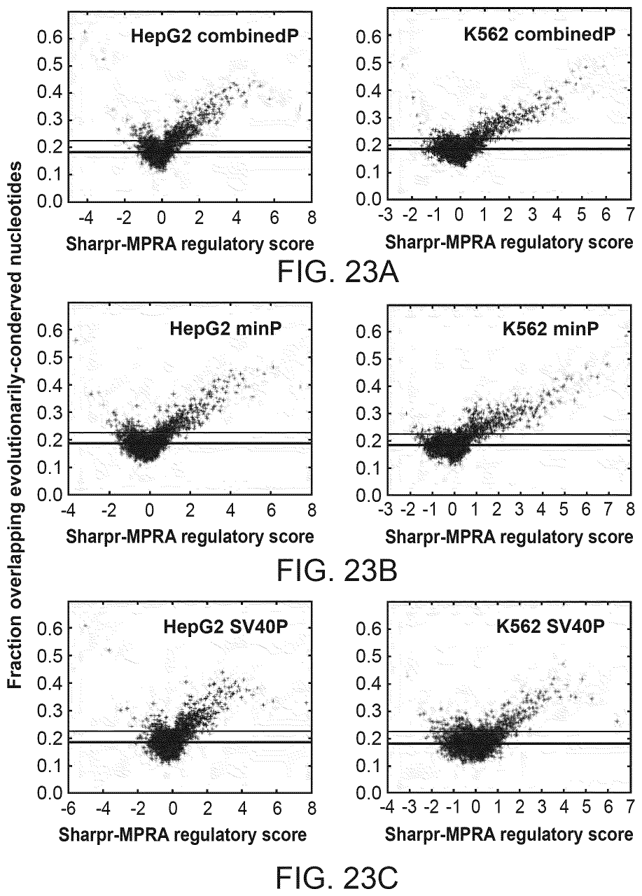

The results are also compared to evolutionarily conserved elements across 29 mammals, and enrichment is found for both activating and repressive nucleotides (FIG. 3c, and FIGS. 12c and 23), also supporting that Sharpr-MPRA captures functional nucleotides in tiled regions.

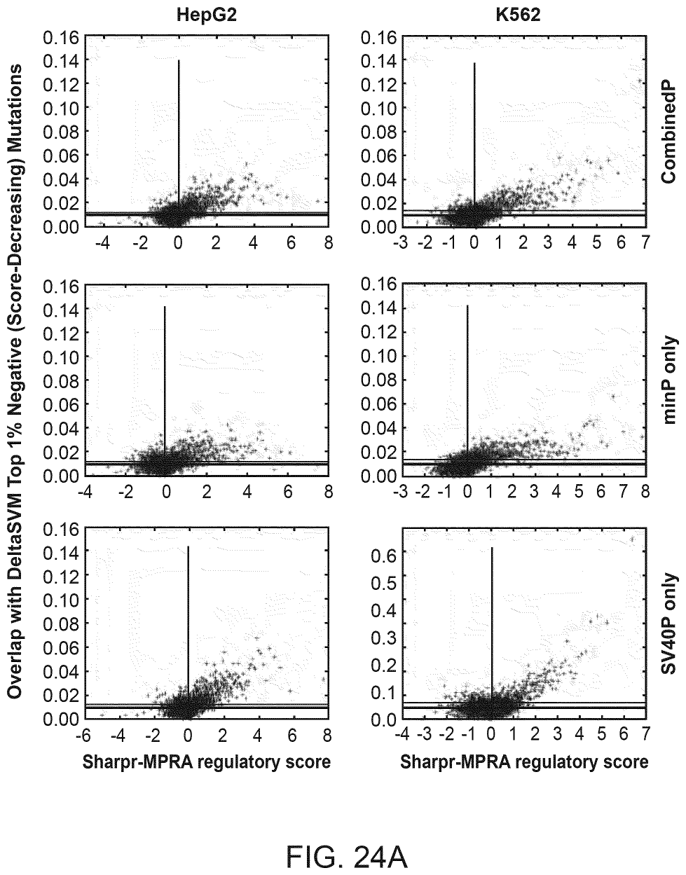

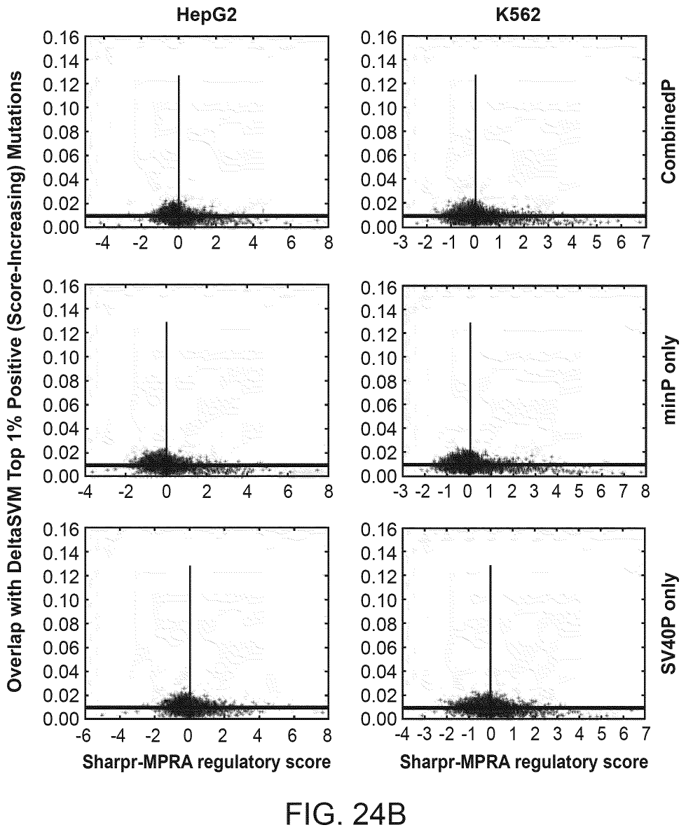

K-mer-based DeltaSVM predictions of nucleotides expected to have regulatory effects when mutated also agreed with the activating nucleotides (FIG. 24 and Methods). However, DeltaSVM predictions did not capture the repressive nucleotides, even though the latter agreed with both conserved nucleotides and CENTIPEDE motifs (FIG. 3c).

Sharpr-MPRA Captured Regulatory Bases at High Resolution

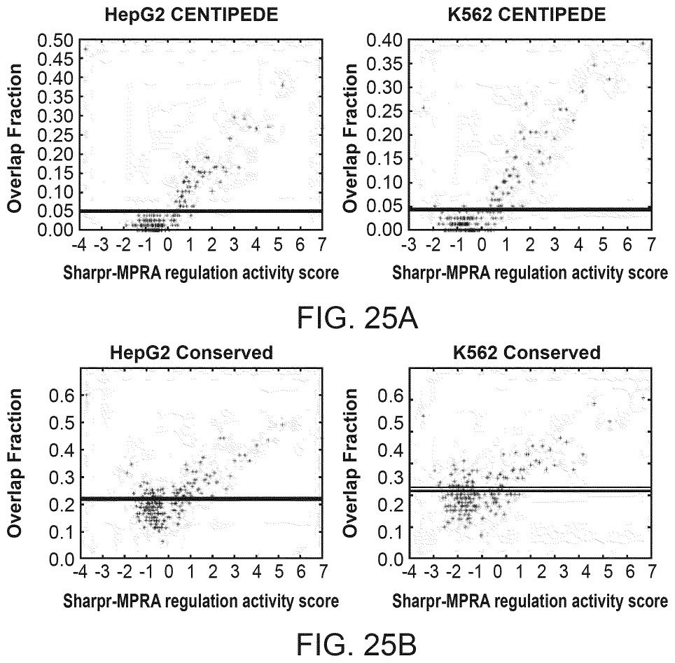

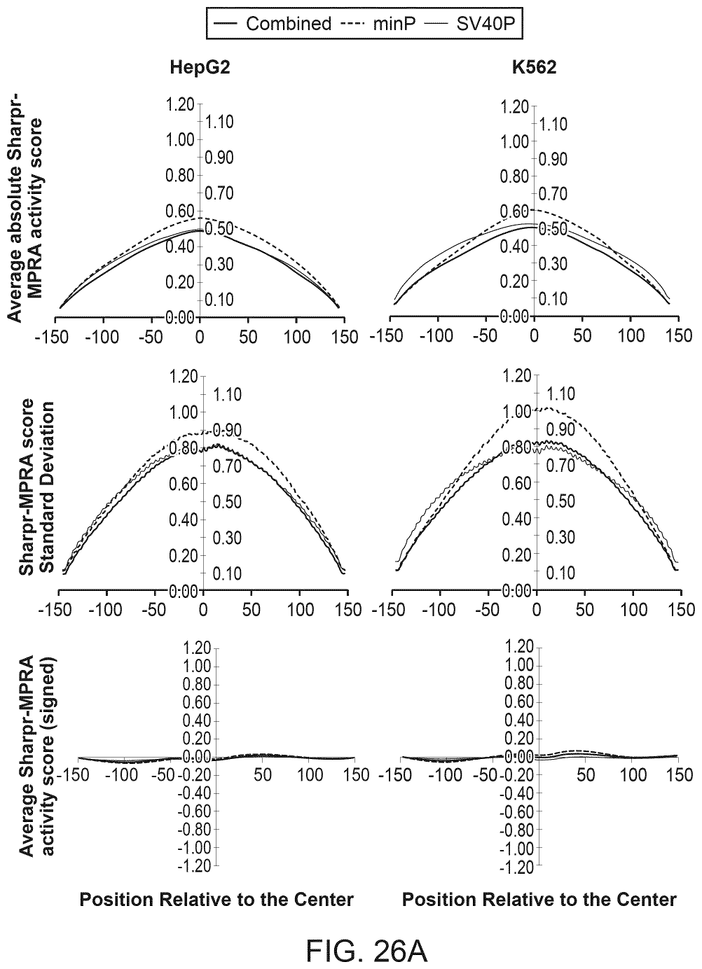

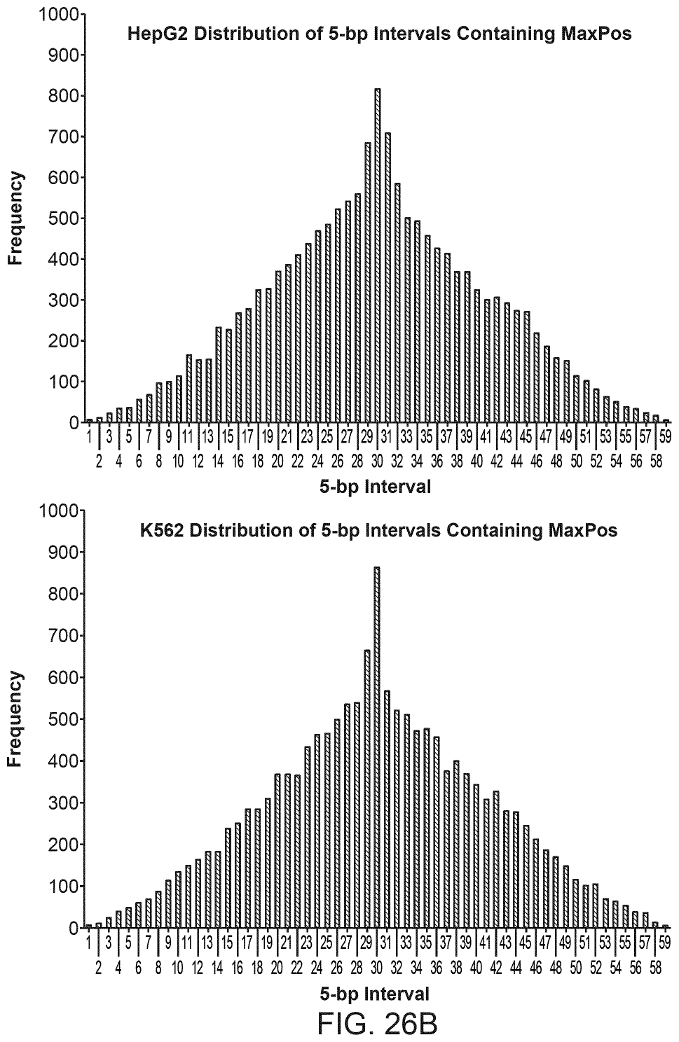

Next, whether the inferences capture high-resolution information in tiled regions is evaluated. It is confirmed that CENTIPEDE motif and conserved element enrichments also held when focusing on MaxPos nucleotides (FIG. 25), a substantial fraction of which disagreed with DNase-site center locations (about 66% of MaxPos nucleotides were outside the 41 central nucleotides, and about 44% were outside the 81 central nucleotides; FIG. 26).

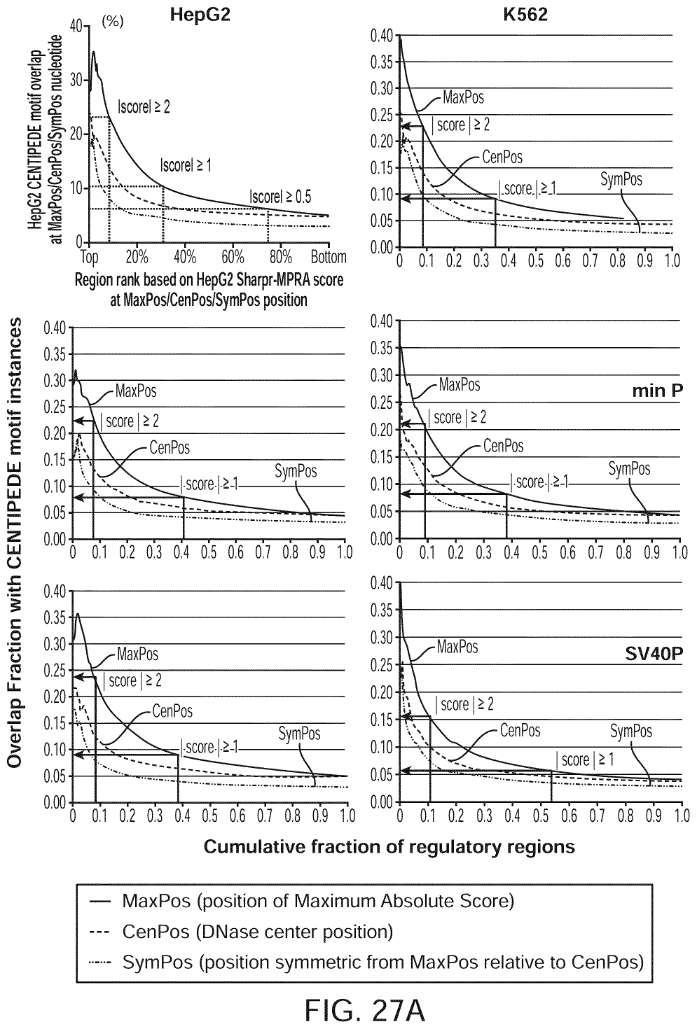

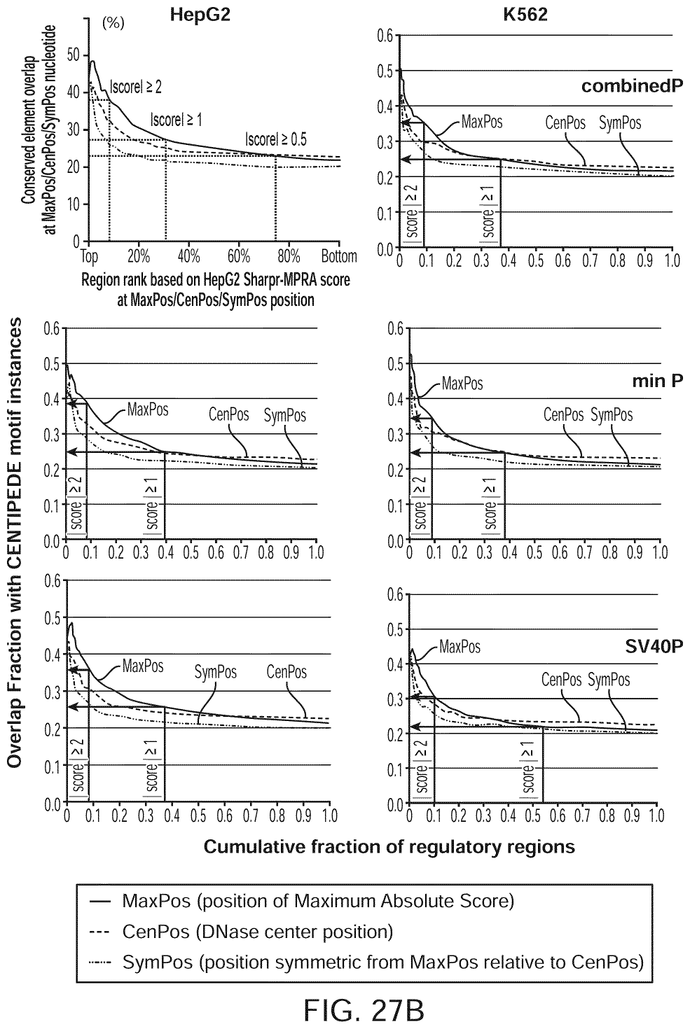

The motif and conserved element enrichments of MaxPos nucleotides are compared to those of DNase site center position (CenPos) nucleotides and their symmetric position (SymPos) nucleotides (equidistant from DNase site centers) (FIG. 3b). The CenPos comparison was particularly stringent, given the higher activity expected at central nucleotides and the better ability to detect activity with more overlapping constructs. Despite these biases favoring central positions, MaxPos nucleotides were substantially more enriched than CenPos nucleotides for both CENTIPEDE motifs and conserved elements, in both HepG2 and K562 cells, for all ranks of high activation or repression (FIG. 3d and FIG. 27). By contrast, their SymPos nucleotides showed substantially lower enrichments than MaxPos nucleotides for all metrics, indicating that MaxPos enrichments do not stem from distance biases.

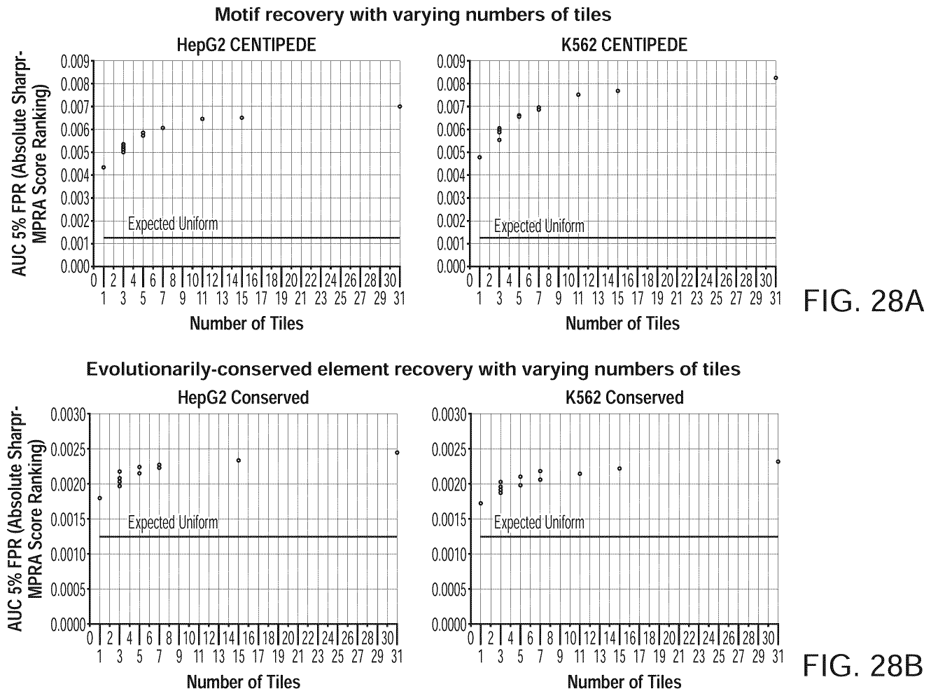

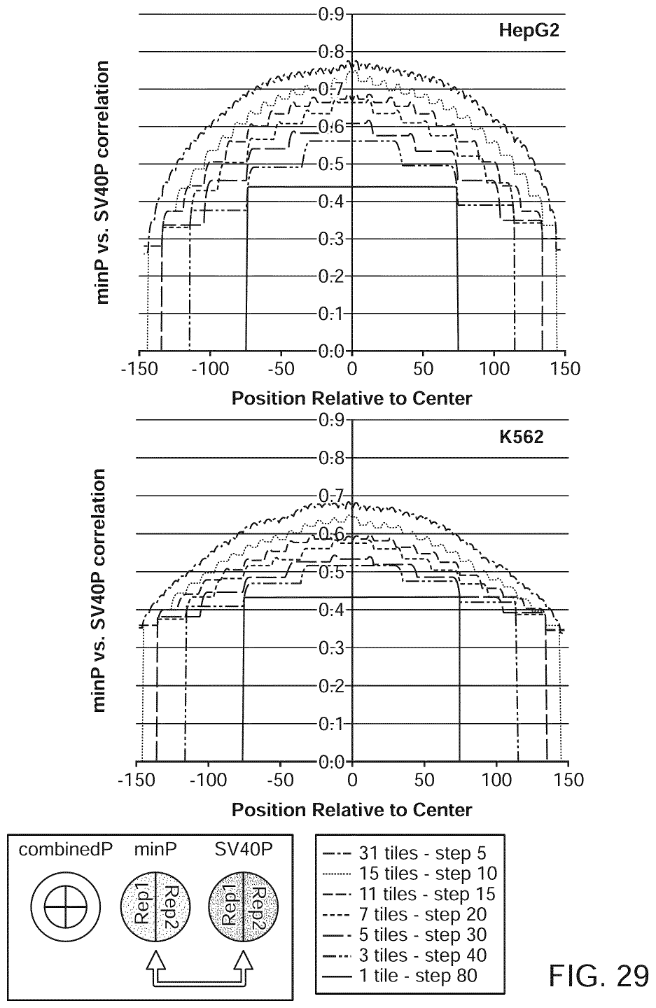

The effect of tiling density on functional element recovery and replicate correlation is evaluated, using increasingly spaced subsets of the reporter constructs. Higher density led to stronger CENTIPEDE motif and conserved element enrichments (FIG. 28) and to higher correlation between replicates (FIG. 29). Saturation was not reached at the 5-bp level used here, indicating that smaller offsets might further increase discovery power, balanced by more constructs per region and thus potentially fewer tiled regions.

Cell-Type-Specific Activating and Repressing Motifs

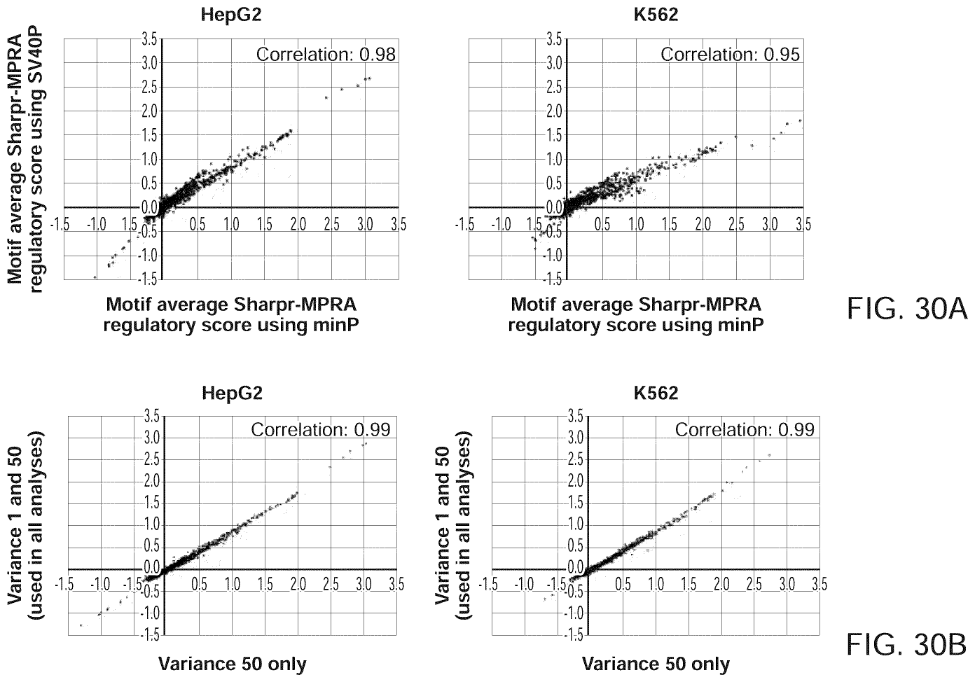

The Sharpr-MPRA results are used to evaluate a compendium of 1,934 understood and predicted regulatory motifs. For each motif, an average motif score is computed (across all central motif positions), and separately an “activating” and a “repressive” enrichment score (based on its enrichment in nucleotides with scores ≥1 and ≤−1, respectively) for each cell type (Methods). Motif scores were largely unchanged between experiments with minP and SV40P, and when using the high-variance prior parameter (≥about 0.95 correlation; FIG. 30).

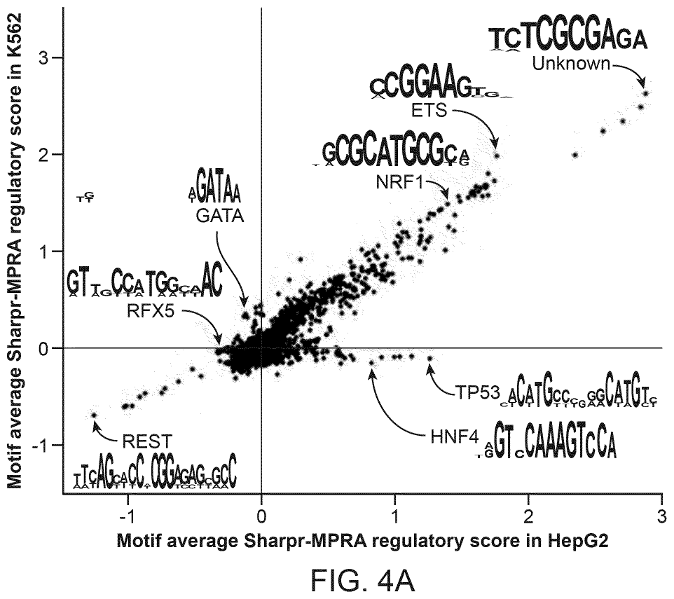

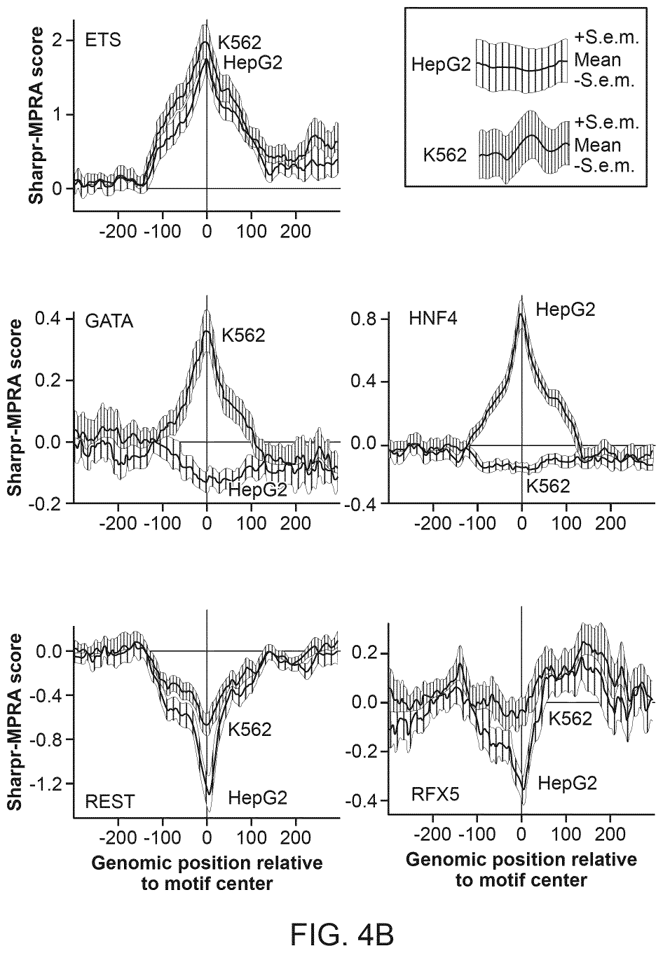

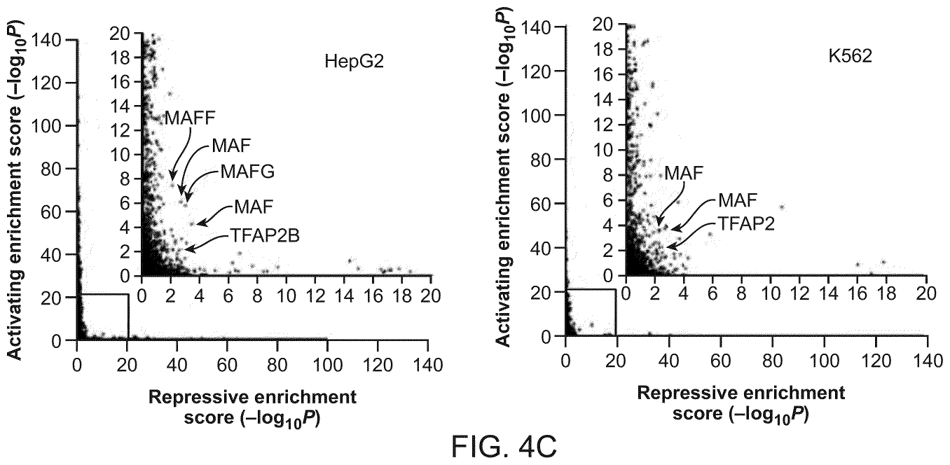

Most motifs showed similar average scores between the two cell types (FIG. 4a). For example, motifs for the ETS and NRF1 regulators showed among the strongest activating average scores in both HepG2 and K562 cells, and repressor REST motif showed the most repressive average score in both. It is found the most activating average score in both cell types for variants of the TCTCGCGAGA palindrome, which was present in the compendium based on its de novo discovery in chromatin immunoprecipitation-sequencing (ChIP-seq) experiments for diverse regulators (including NR3C1, BRCA1, ETS, CHD2, and ZBTB33, and so forth). This motif lacks a well-established regulator in vivo, despite support for its importance from strong evolutionary conservation, high nearby gene expression, and other experimental and bioinformatics evidence.

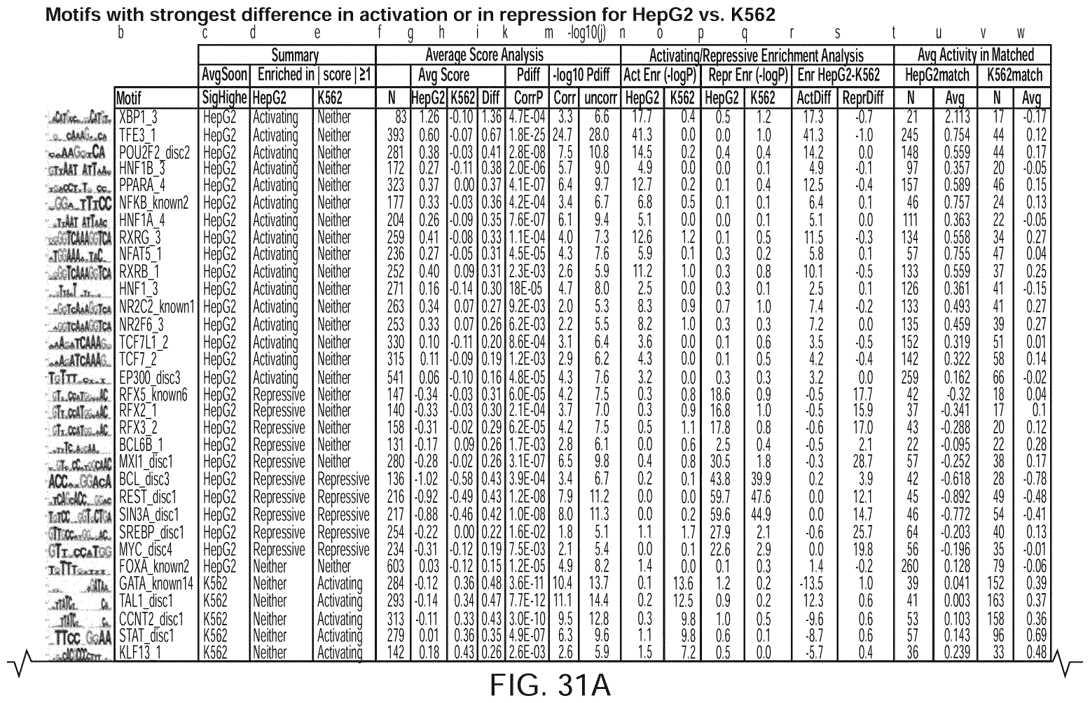

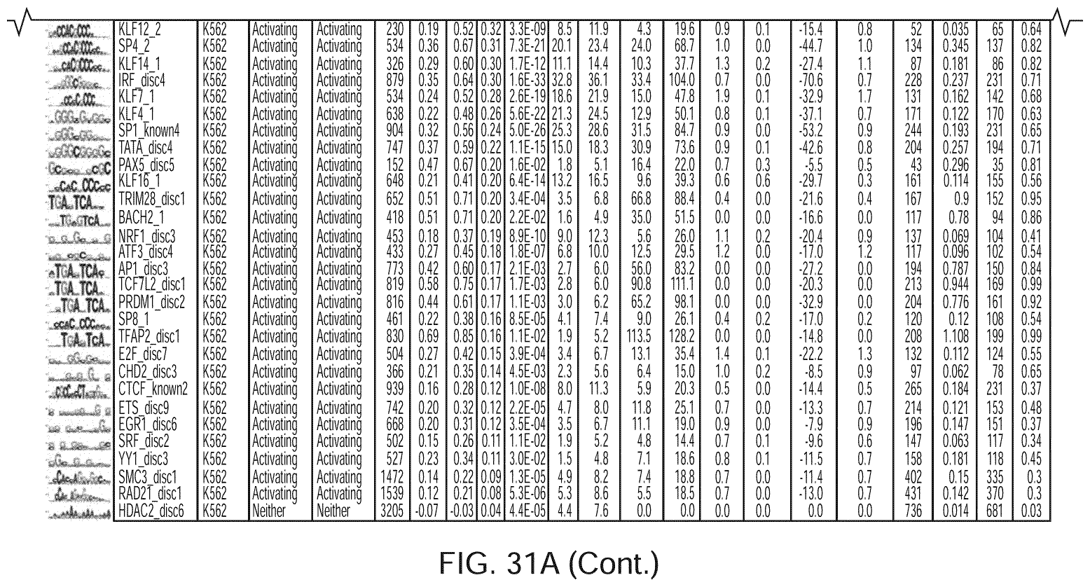

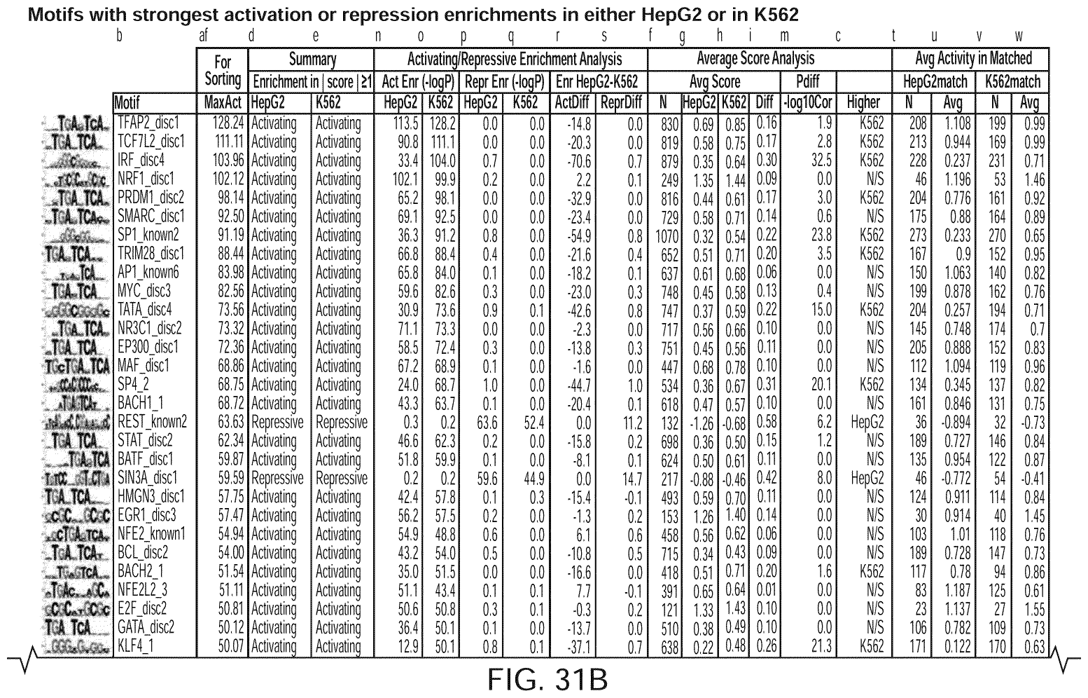

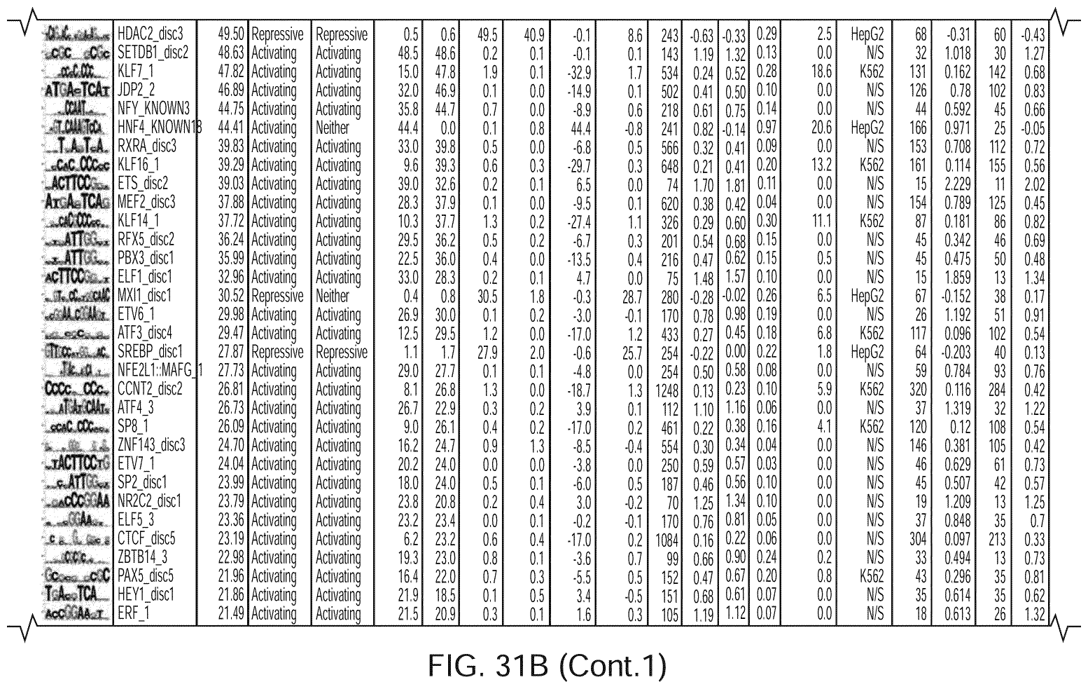

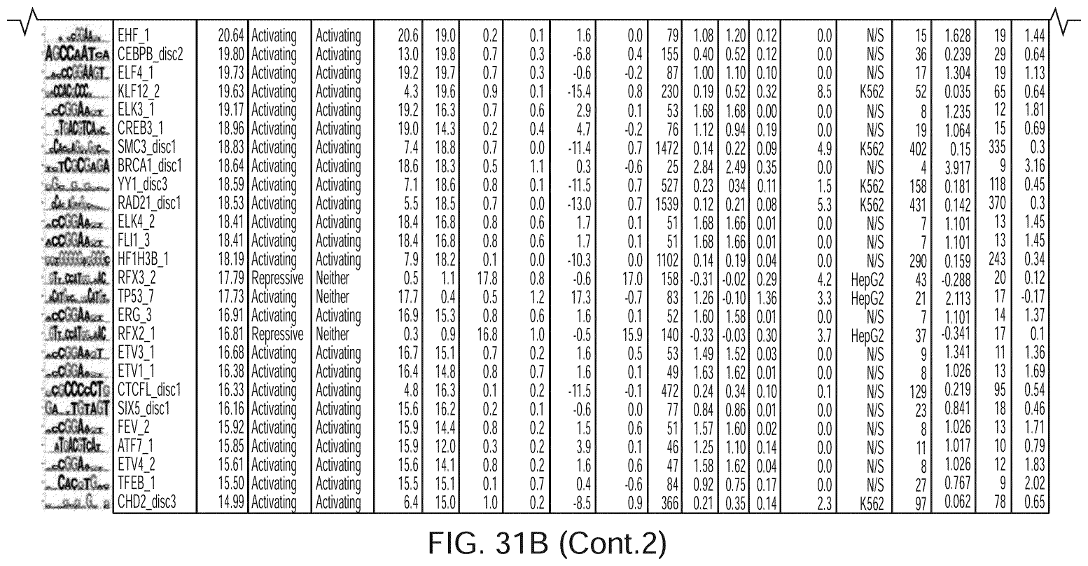

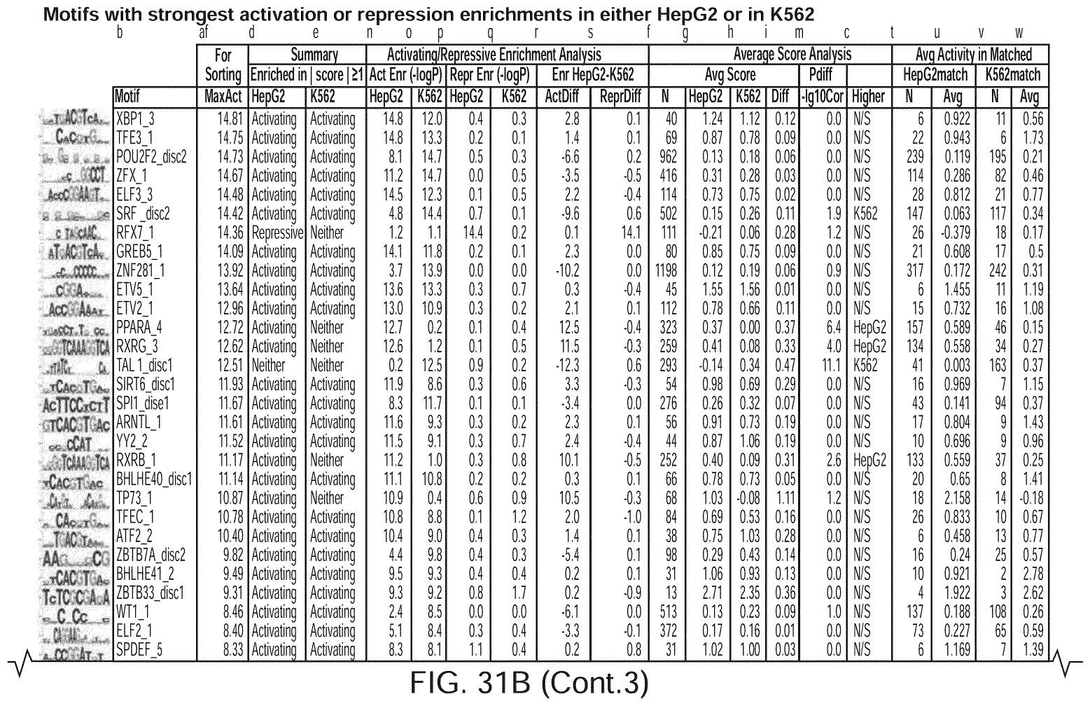

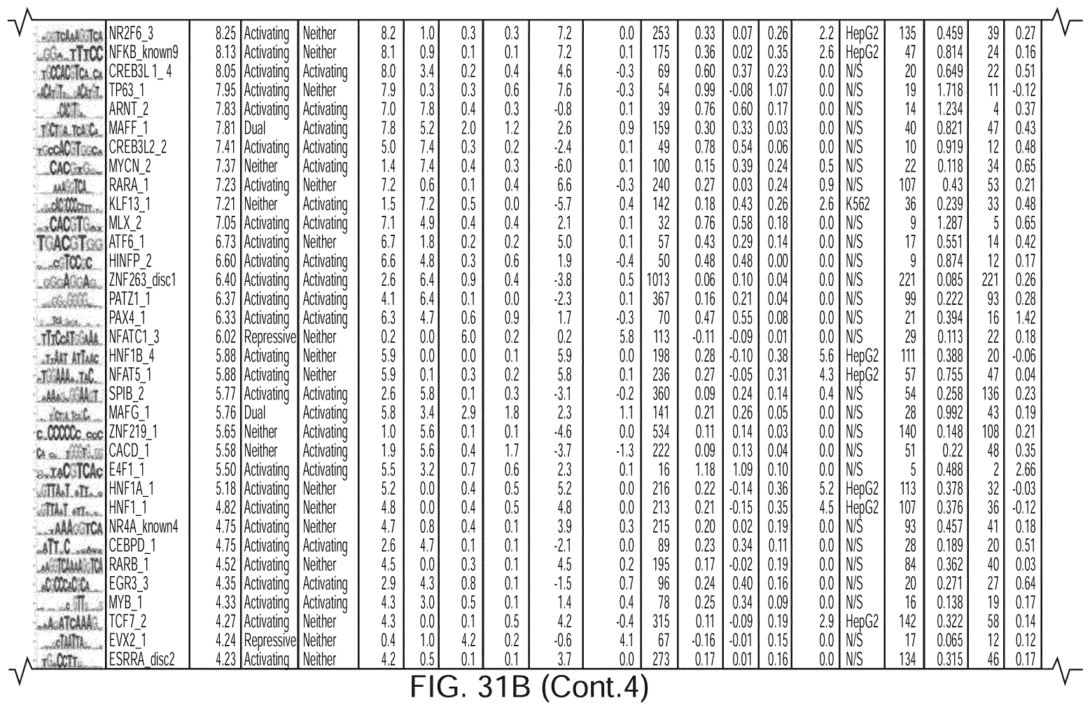

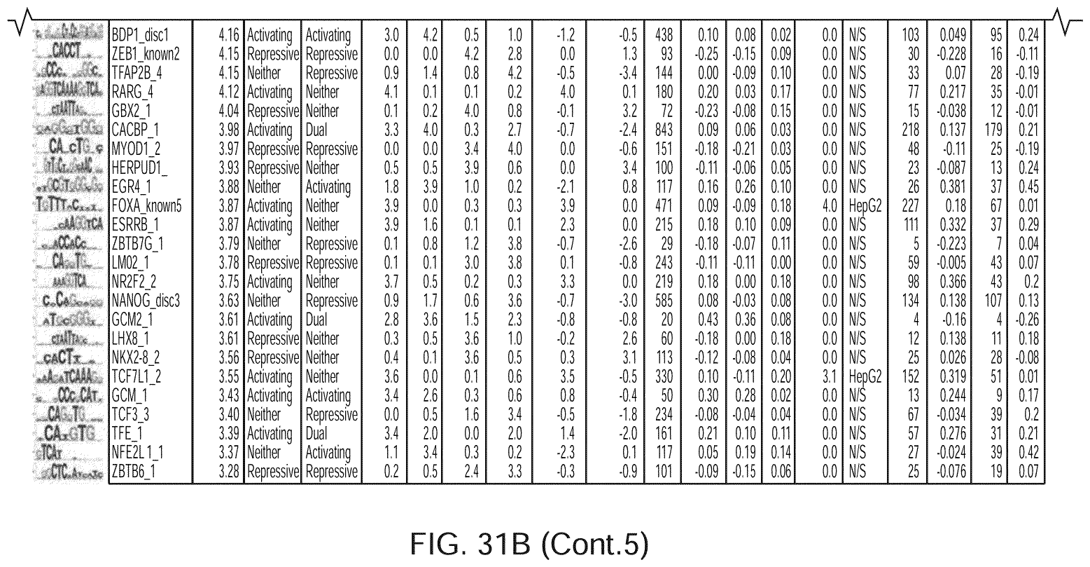

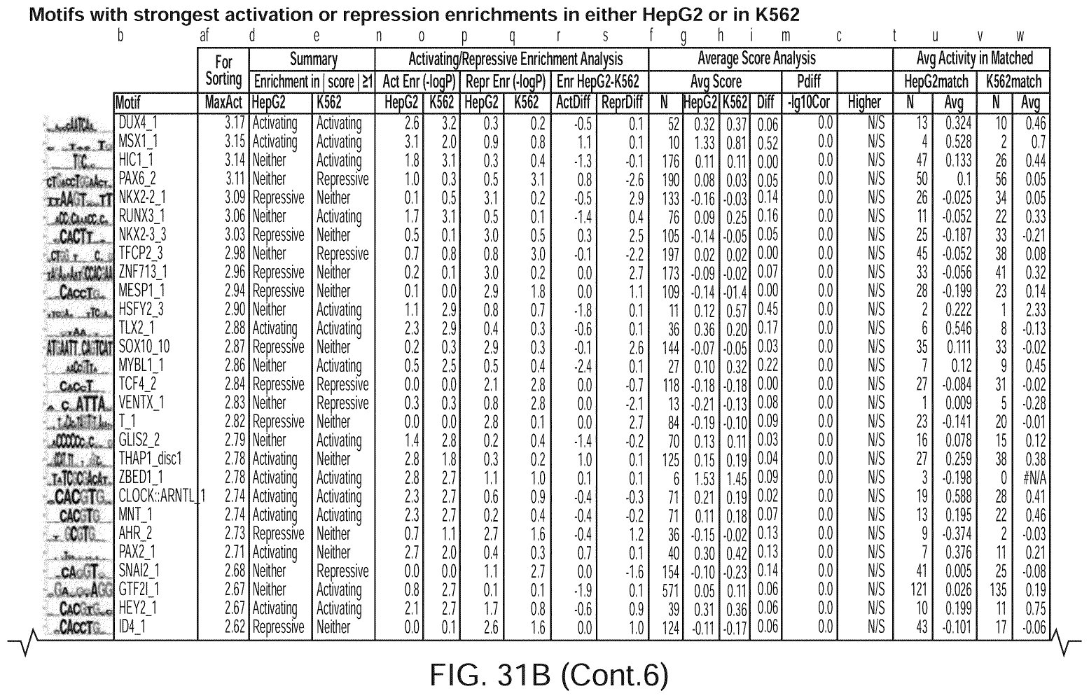

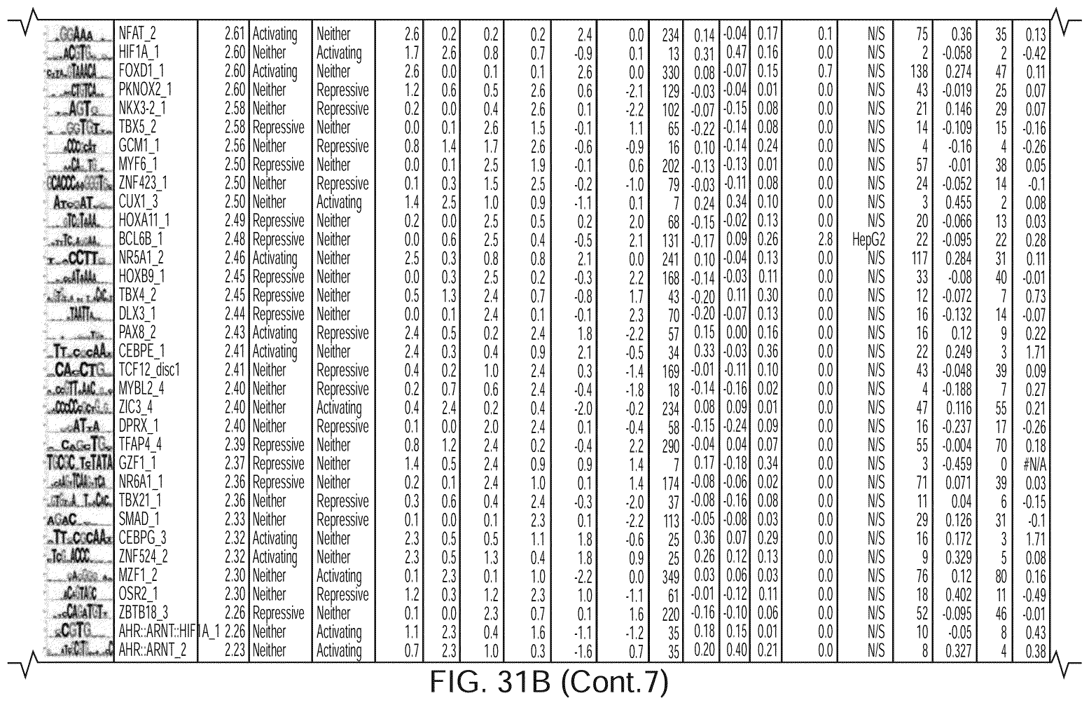

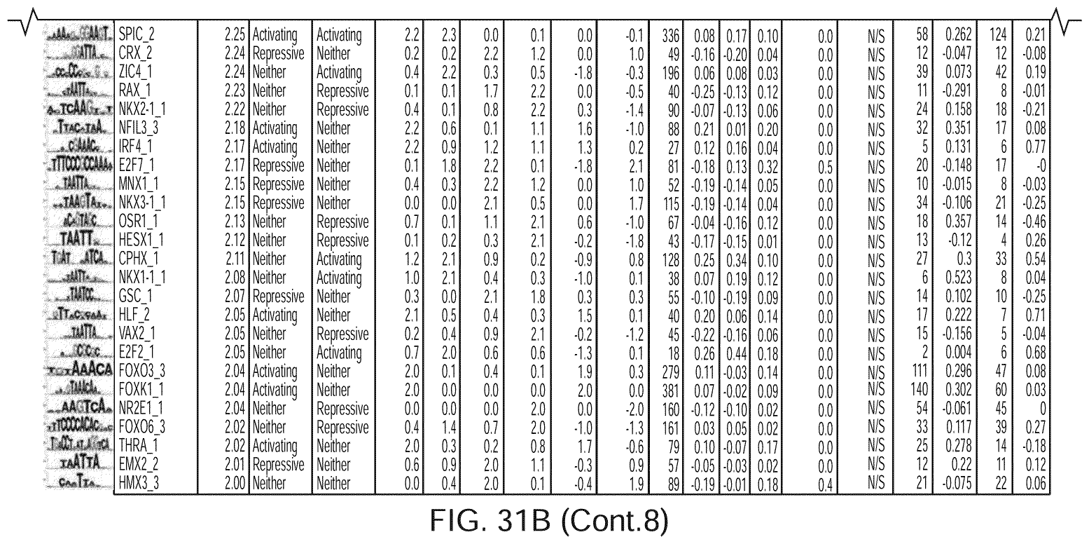

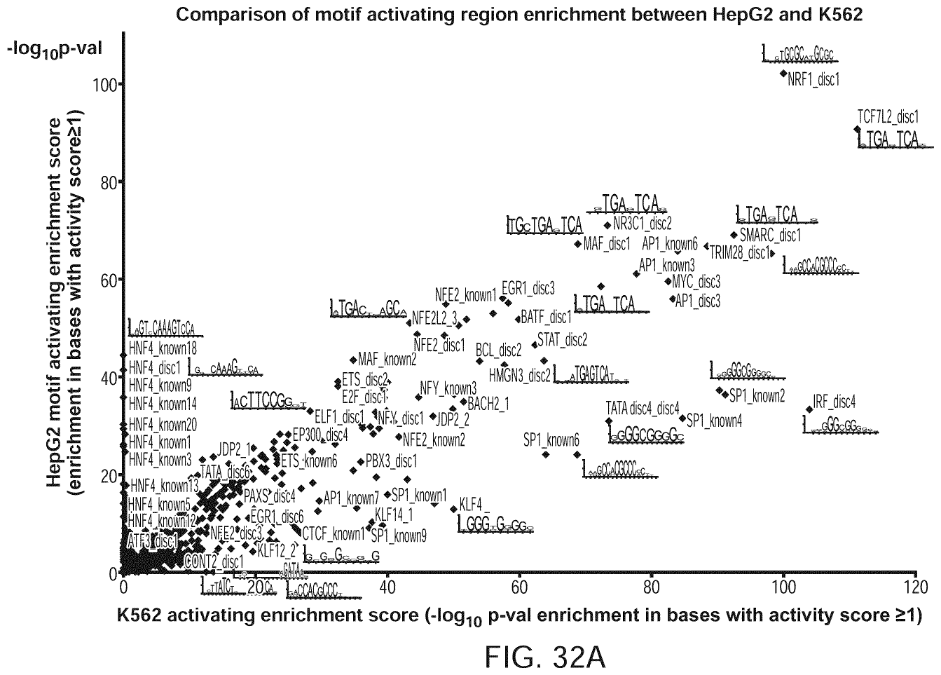

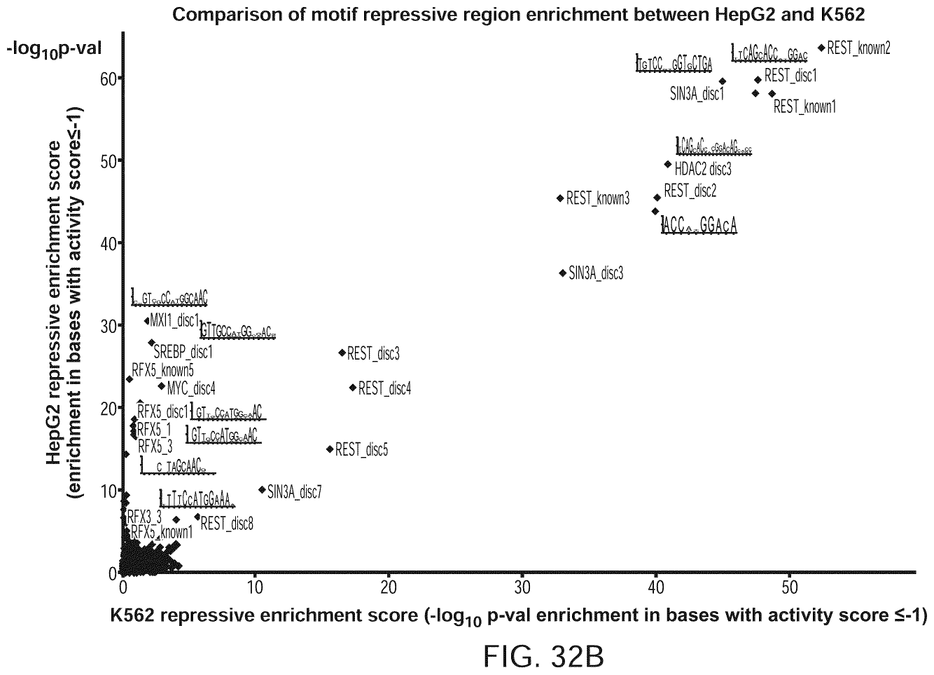

A subset of motifs showed significant differences (using a paired t-test) in scores between HepG2 and K562 cells (FIG. 31a). Significantly different activating motifs included HNF4, RXRA, PPARA, HNF1A, HNF1B and FOXA in HepG2 cells, consistent with liver-related roles, and GATA, SP1 and KLF in K562 cells, consistent with K562 cell roles (FIG. 32a). Significantly different repressive motifs included multiple RFX motifs in HepG2 cells (FIG. 32b), consistent with other evidence for one enhancer.

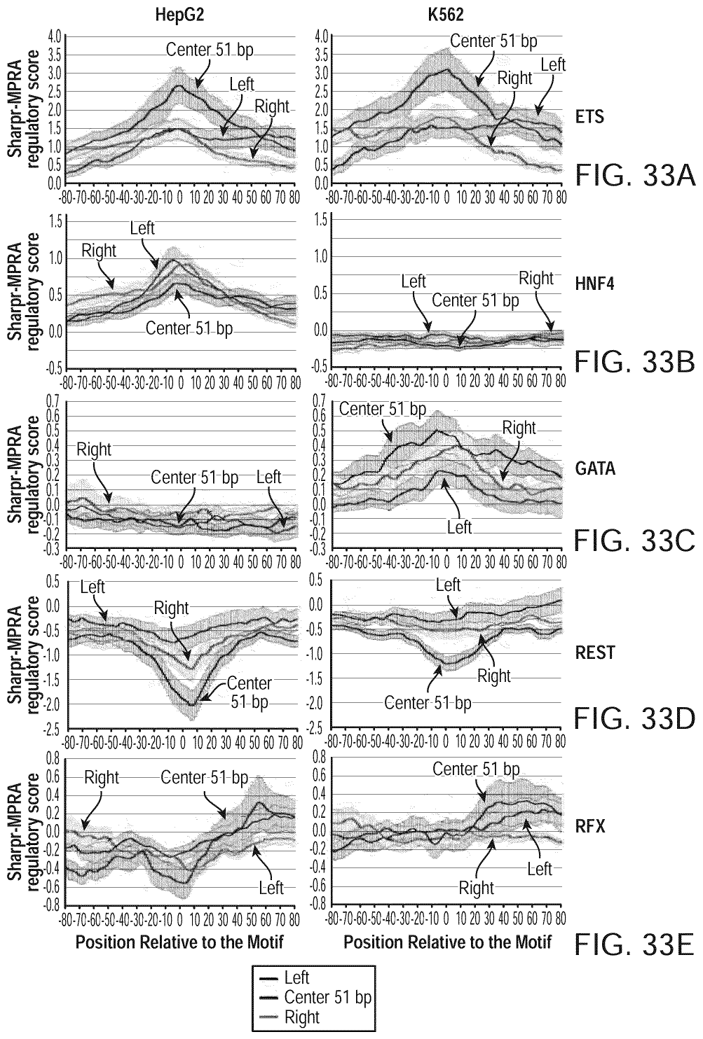

Cell type-specificity was also reflected in the position-specific aggregated distribution of activity scores surrounding all instances of these motifs (FIG. 4b). Activating motifs (for example, ETS, GATA and HNF4), showed a positive peak surrounding their instances, and repressor motifs (for example, REST and RFX) showed a negative peak, in each case matching the expected cell types. These patterns were stronger when motifs occurred in more central positions (FIG. 33), given the higher reporter coverage and absolute activity of central positions.

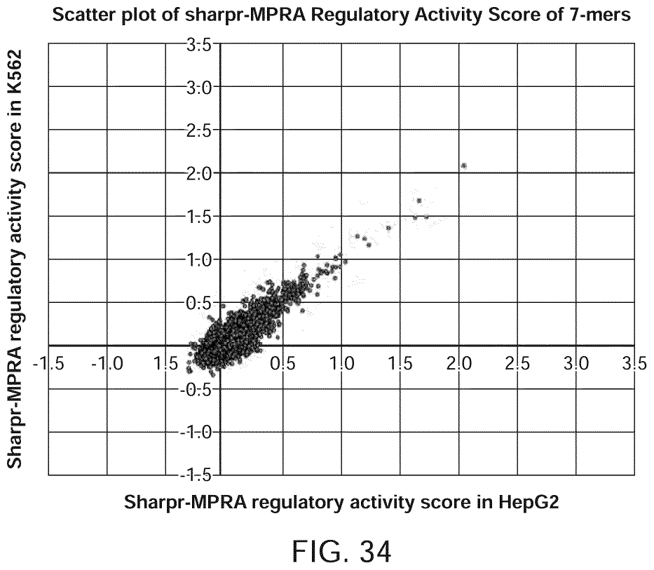

The motif compendium resulted in more activating than repressive motif scores, both based on average scores (FIG. 4a), and based on activating versus repressive enrichment scores (about 511 vs. about 117 in HepG2 cells; about 474 vs. about 79 in K562 cells, respectively, at an uncorrected P value of about 0.01; FIG. 4c, FIG. 31b). The higher number of activating motifs also held for average scores of all 7-mer sequences (FIG. 34), indicating it is not an ascertainment bias in the compendium used.

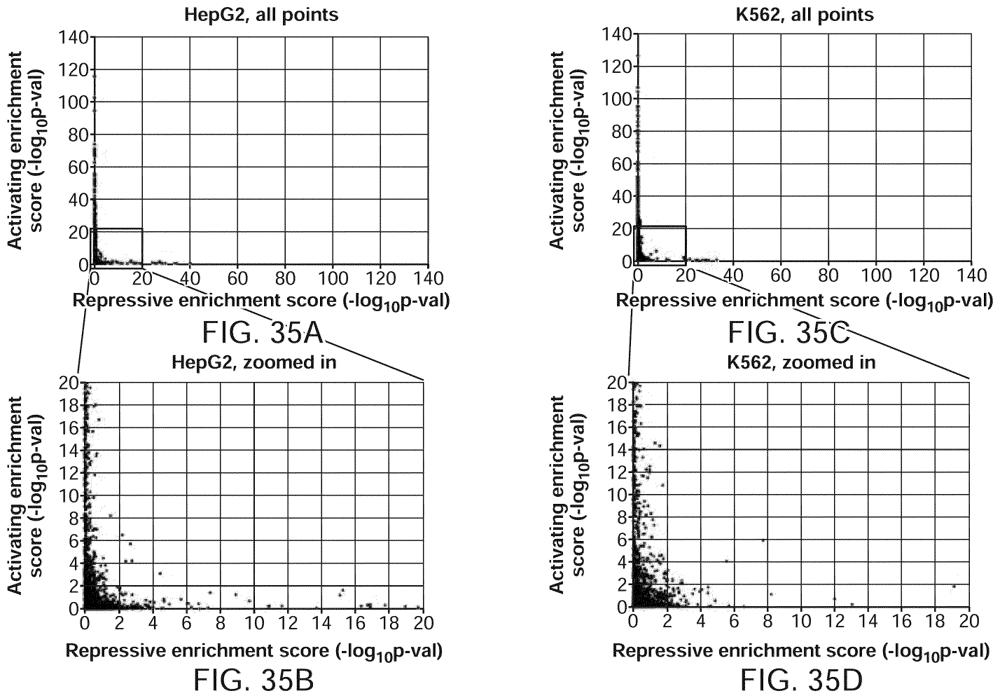

A small number of “dual-role” motifs showed signatures of both activating and repressive function in the same cell type (14 in HepG2 and 15 in K562 cells, of which six were in common). These included several motifs for MAF proteins, consistent with reports of both activation and repression and different motifs of the AP-2 family. Alternative cutoffs (top 5% activating and repressive nucleotides) also resulted in very few dual-role motifs (6 in HepG2 and 9 in K562 cells; FIG. 35).

Enrichment of ERV1 Repeats in Activating Nucleotides

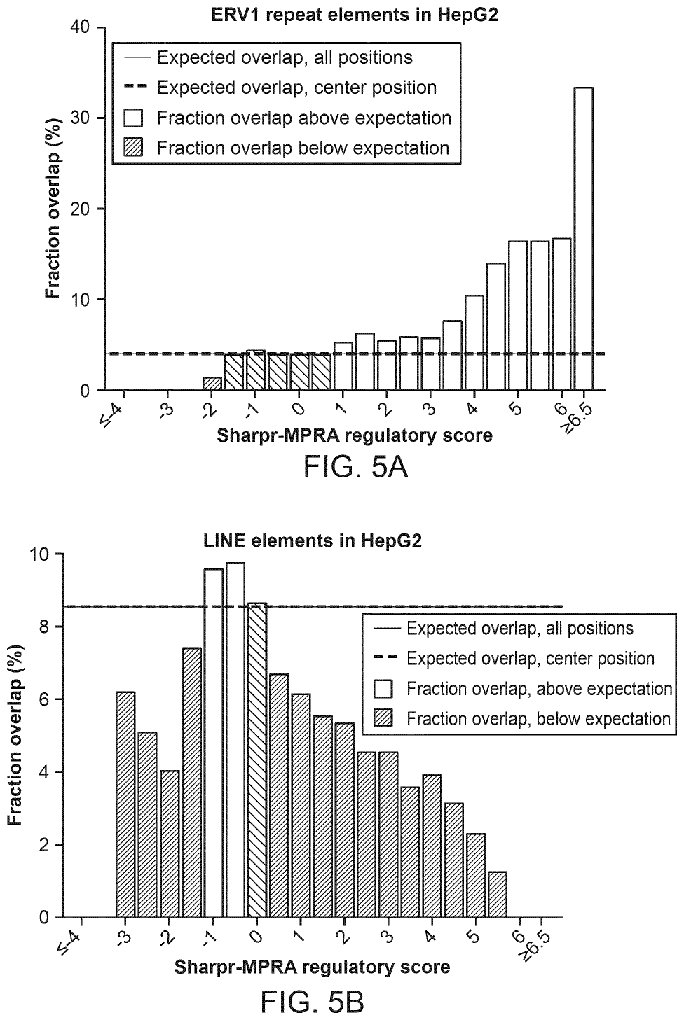

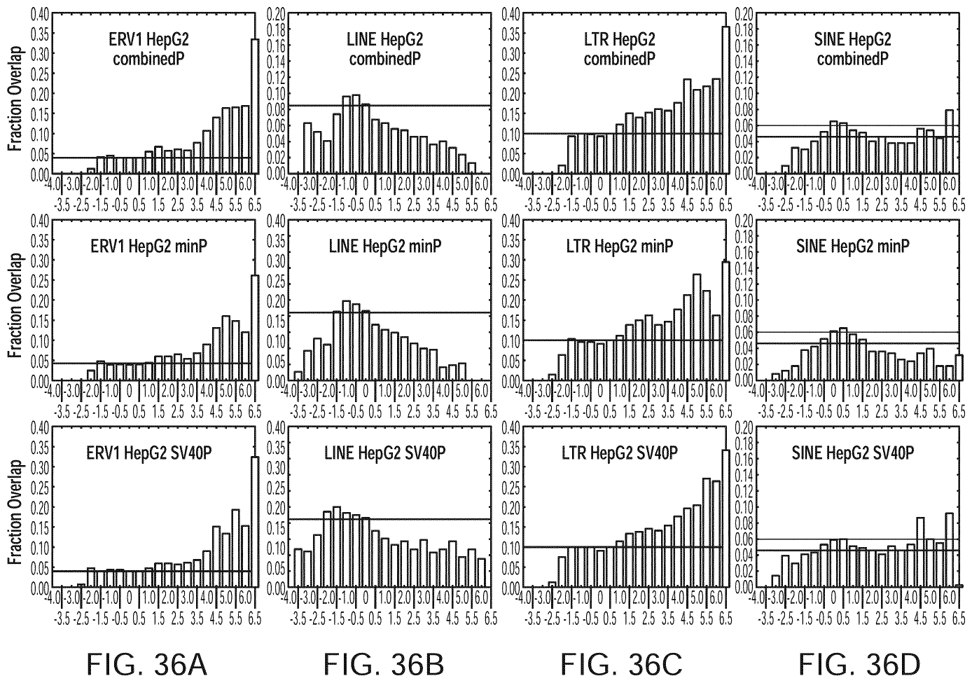

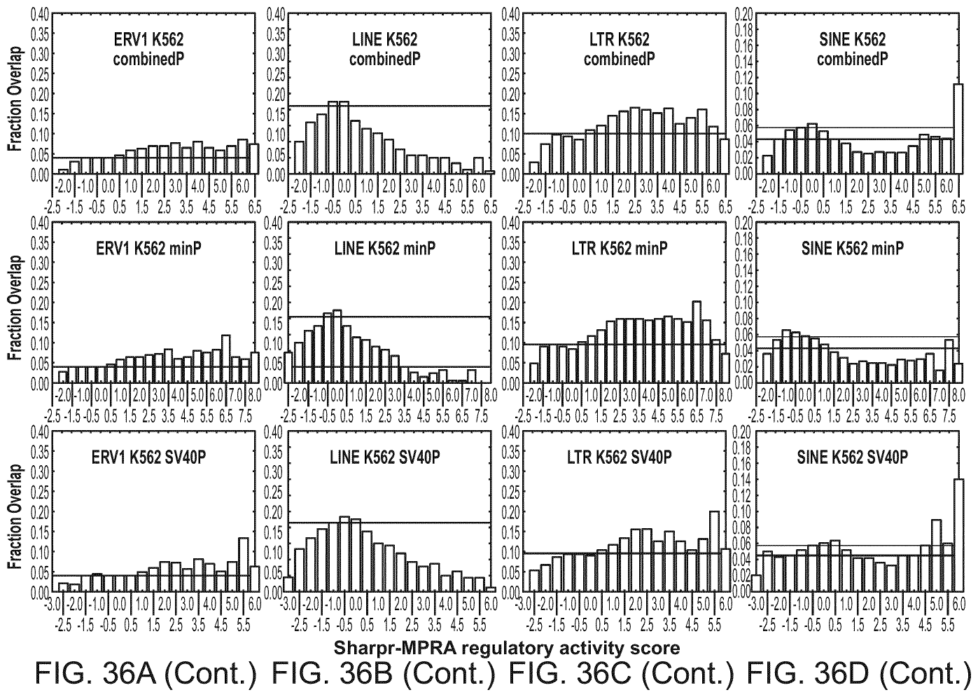

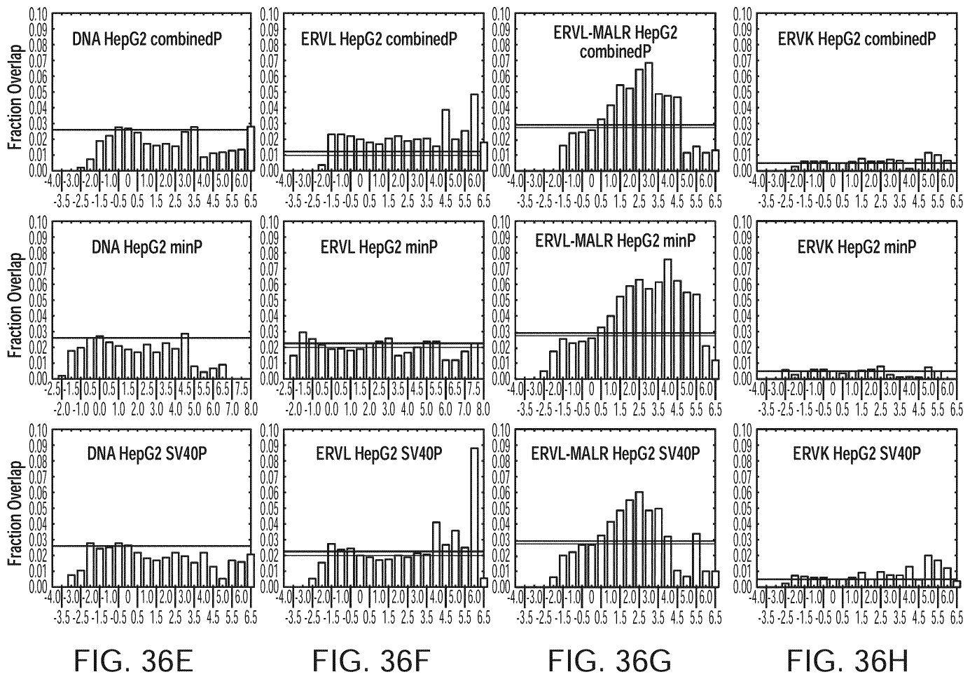

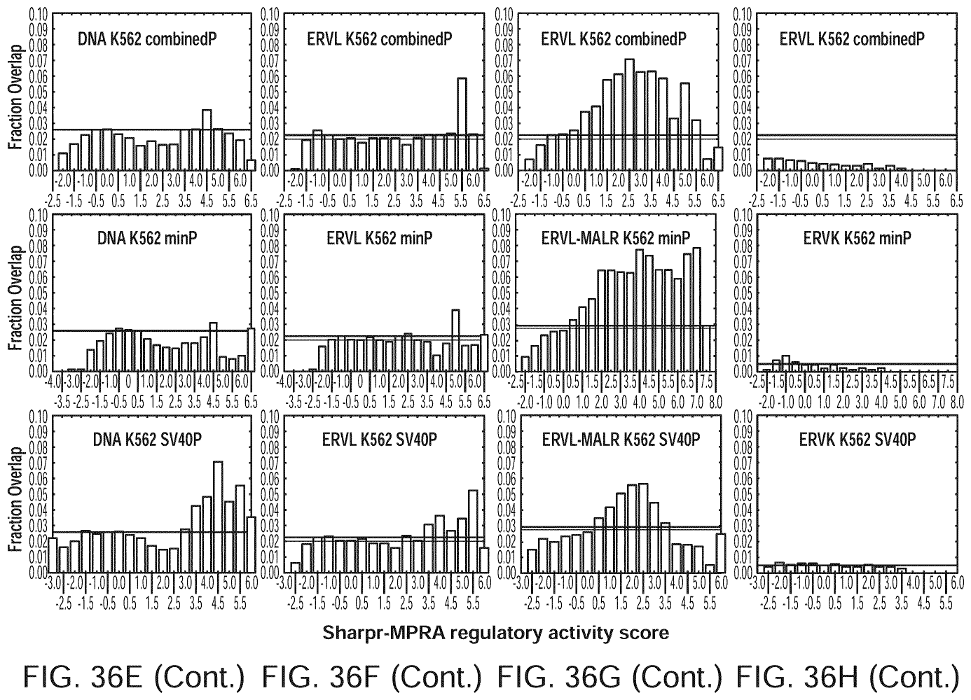

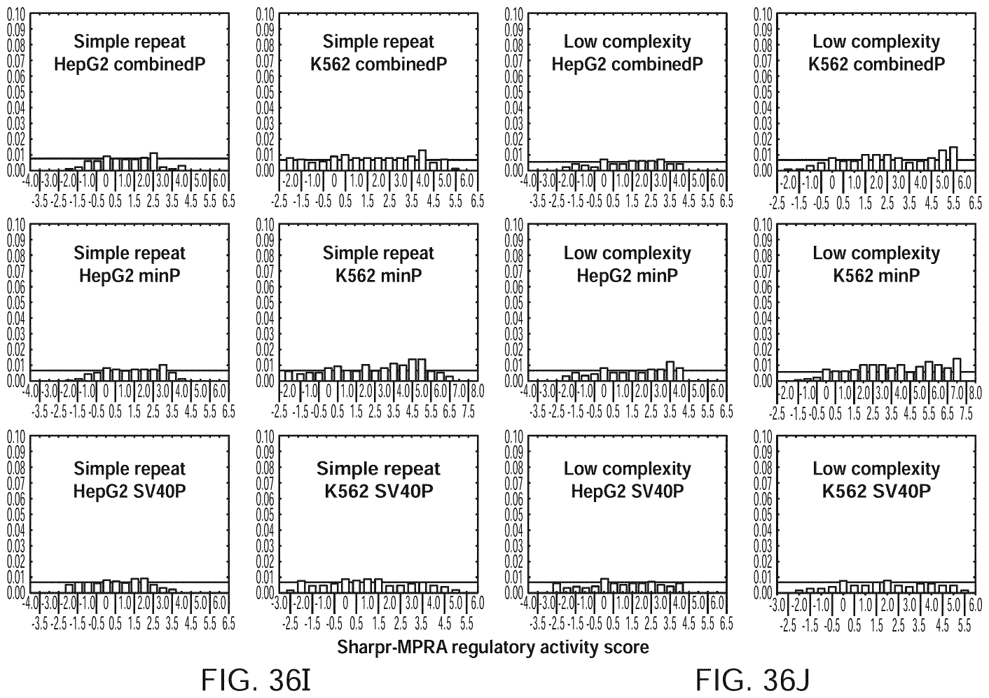

The most strongly activating nucleotides in HepG2 cells showed substantial enrichment for long terminal repeat (LTR) elements (FIG. 5 and FIG. 36), consistent with reports of their ability to drive gene expression. This helps to explain why conserved elements were less enriched in the most extreme activating scores (FIG. 3c), as no LTRs overlapped conserved elements with the most extreme activating scores.

Among LTRs, endogenous retroviral sequence 1 (ERV1) repeats showed the strongest enrichment in HepG2 activating nucleotides (FIG. 5a), overlapping about 33% of the 820 nucleotides with the highest regulatory scores (≥6.5 bin), versus about 4% expected on average (eightfold enrichment). Regulatory roles are hypothesized for ERV1 repeats, based on TF binding and RNA interference evidence, and the results indicate these repeats can function autonomously and lead to strong episomal expression.

By contrast, long interspersed nuclear elements (LINEs) were strongly depleted in both activating and repressive nucleotides in HepG2 and K562 cells (FIG. 5b and FIG. 36b), indicating repeat-specific regulatory functions. Moreover, ERV1 and other LTR enrichments were weaker in K562-cell than HepG2-cell activating nucleotides (FIG. 36a,c), indicating cell-type-specific repeat functions.

Epigenomic Signatures were Predictive of Reporter Activity

The relationship between regulatory activity scores and endogenous chromatin state is analyzed, afforded by inclusion of all chromatin states (specified in FIG. 11), and both DNase and non-DNase sites in each cell type (by including DNase sites in other cell types).

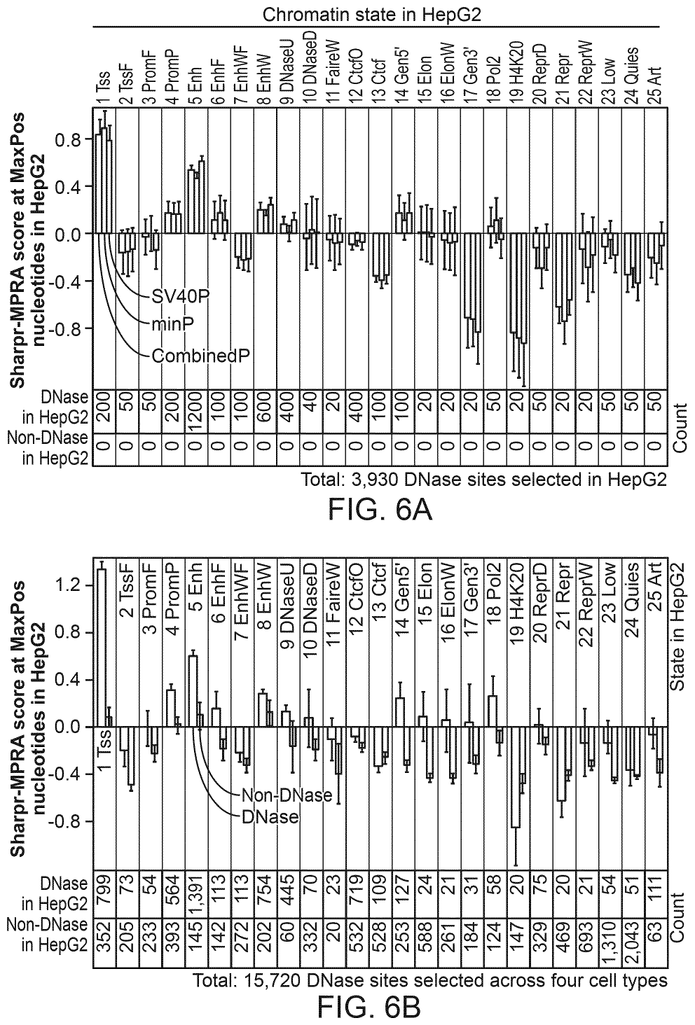

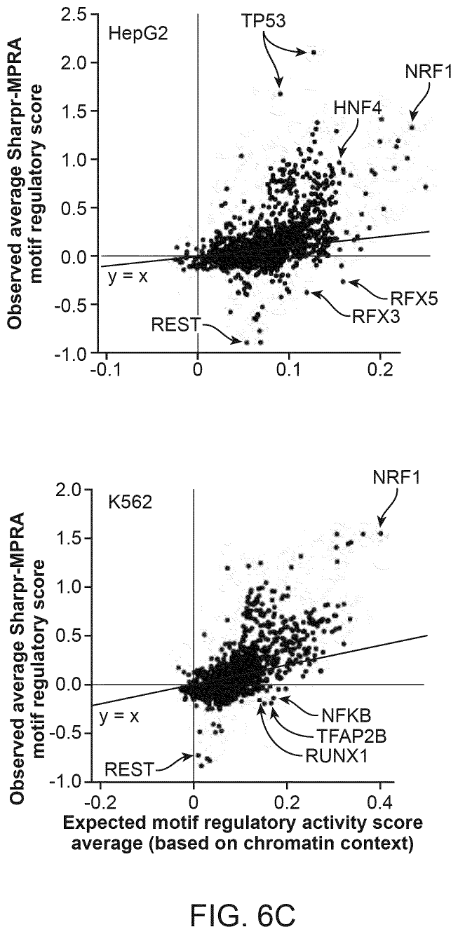

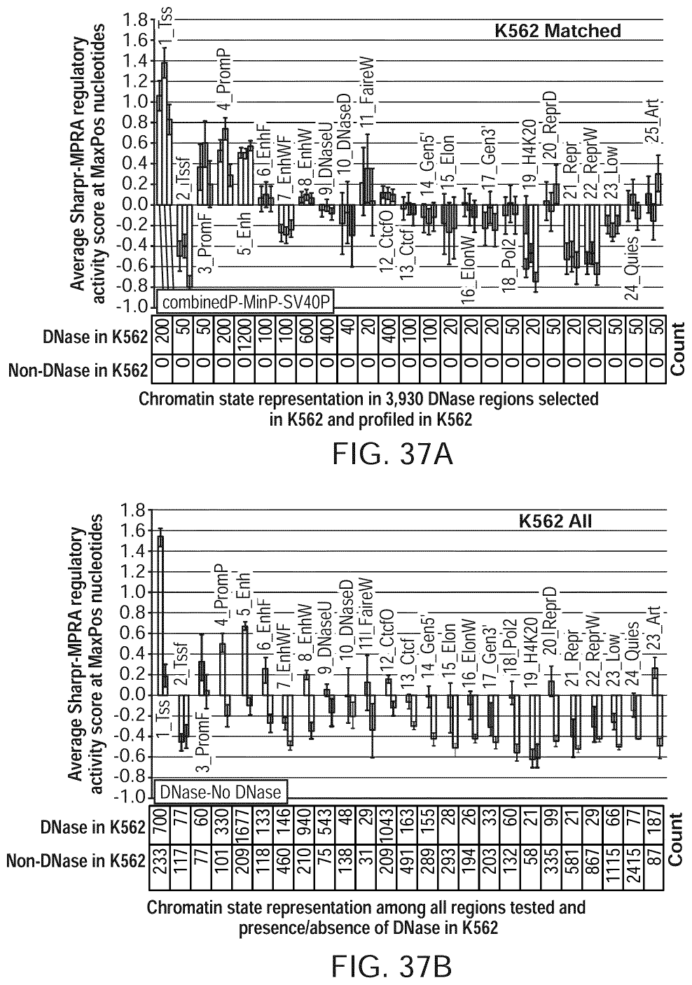

Among DNase sites, endogenous chromatin state was predictive of regulatory function in reporter assays (quantified for each region by its MaxPos Sharpr-MPRA score; FIG. 6a and FIG. 37a). Regions in active promoter or H3K27ac-marked enhancer chromatin states showed higher Sharpr-MPRA activating scores, regions in weak enhancer states showed intermediate activating scores, and regions in Polycomb-associated states showed repressive Sharpr-MPRA scores. Conversely, among genomic locations in the same chromatin state, the DNA accessibility of the region in its endogenous context was predictive of Sharpr-MPRA reporter activity (FIG. 6b and FIG. 37b). Together, these results indicate that the endogenous epigenomic signatures of DNA accessibility and chromatin state each capture information about regulatory function, and that sequence elements in these regions can show consistent activating or repressive regulatory functions outside their endogenous context.

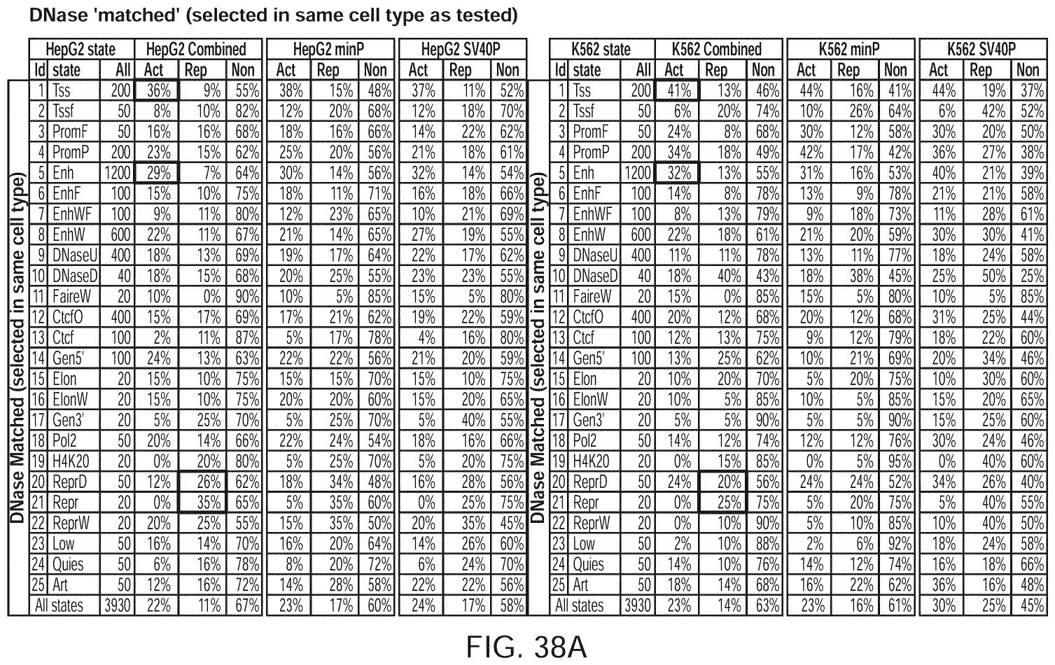

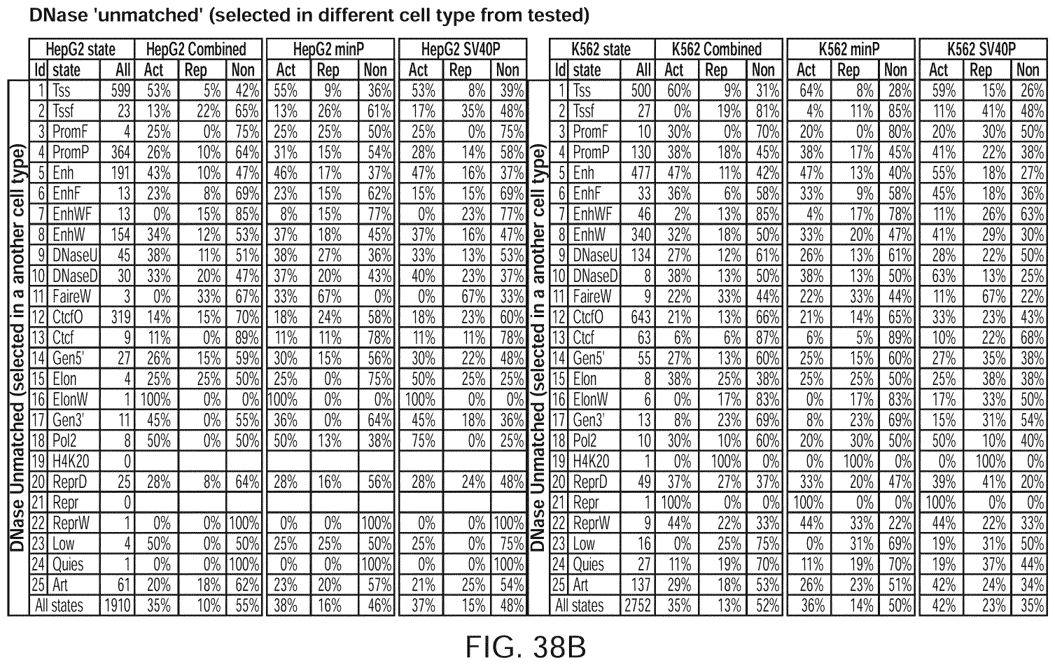

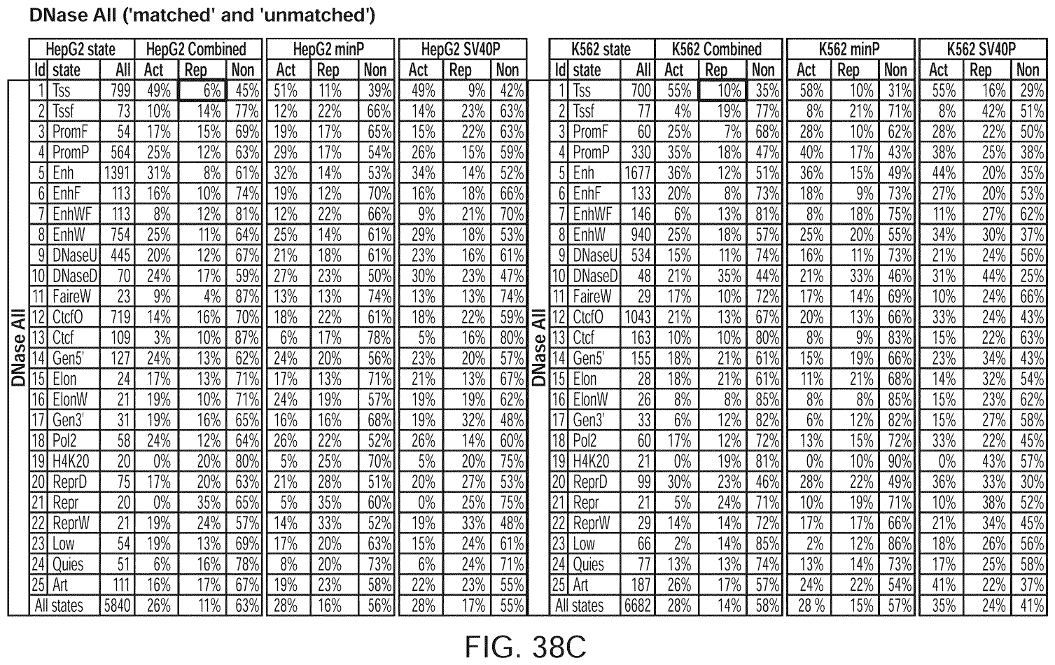

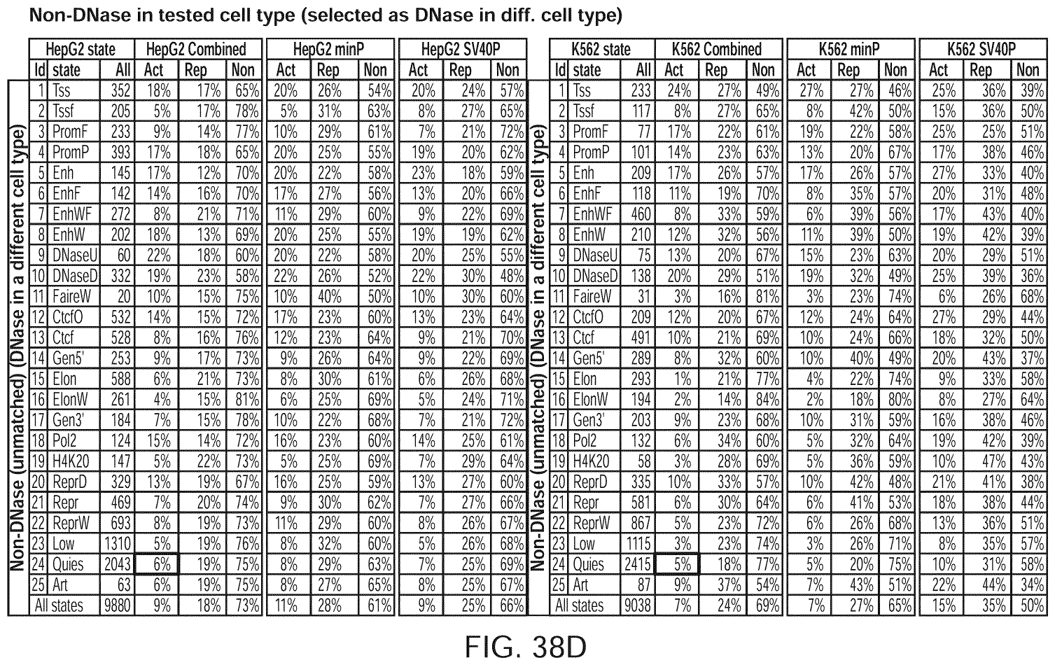

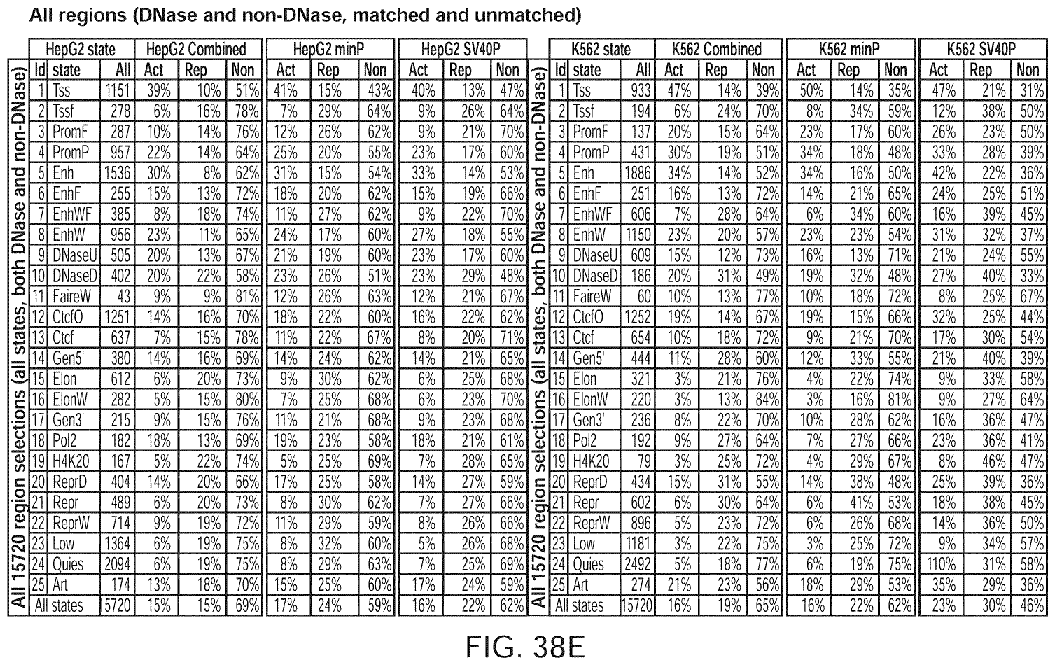

The fraction of regions that showed activating and repressive MaxPos scores varied greatly between chromatin states and DNase classes (FIG. 38). In HepG2 cells, activating regions with scores ≥1 included about 36% of HepG2-selected DNase sites in an active promoter state (Tss) and about 29% in an H3K27ac-marked enhancer state (Enh) (FIG. 38a), compared to about 6% for non-DNase sites in the quiescent states (Quies) (FIG. 38d) (about 41% and about 32% vs. about 5%, respectively, in K562 cells). Repressive regions with MaxPos scores ≤−1 in HepG2 cells included about 29% of HepG2-selected DNase sites in Polycomb repressed states ReprD and Repr (about 21% for K562 cells) (FIG. 38a), compared to about 6% of all DNase sites in the active promoter state (about 10% in K562 cells) (FIG. 38c). These comparisons allowed the estimation of false positive rates for both activating regions (for example, about 6% for HepG2 cells and about 5% for K562 cells) and for repressive regions (for example, about 6% and about 10%, respectively) relative to their respective backgrounds. These estimates are likely conservative, as all regions tested were in DNase sites in at least one cell type, and thus more likely to contain activating or repressive elements than random background nucleotides.

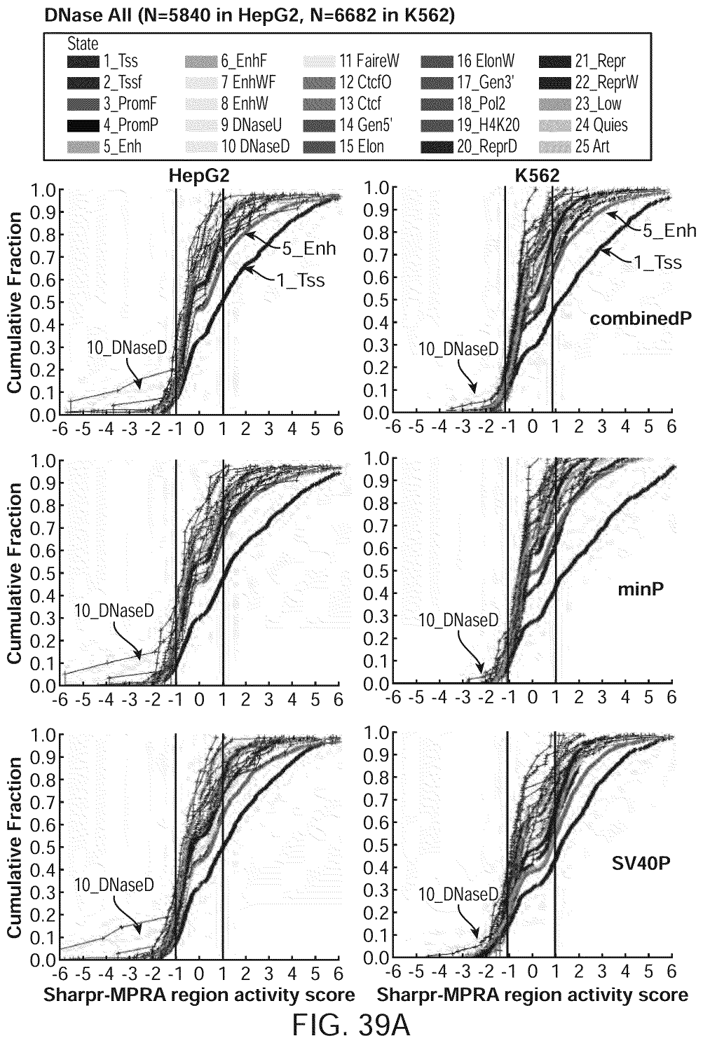

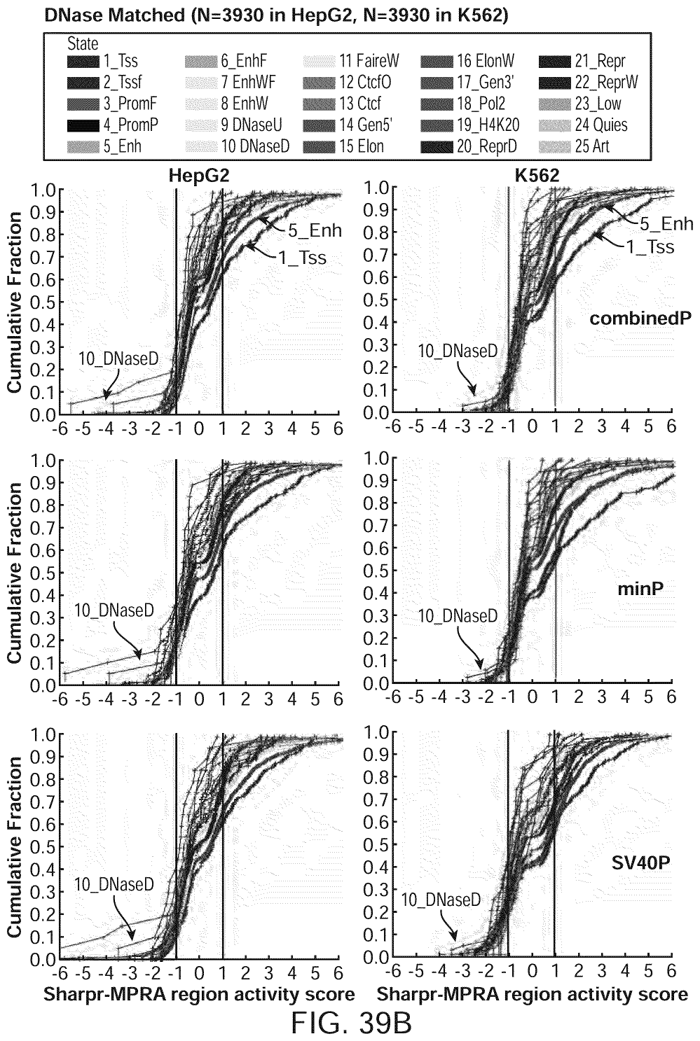

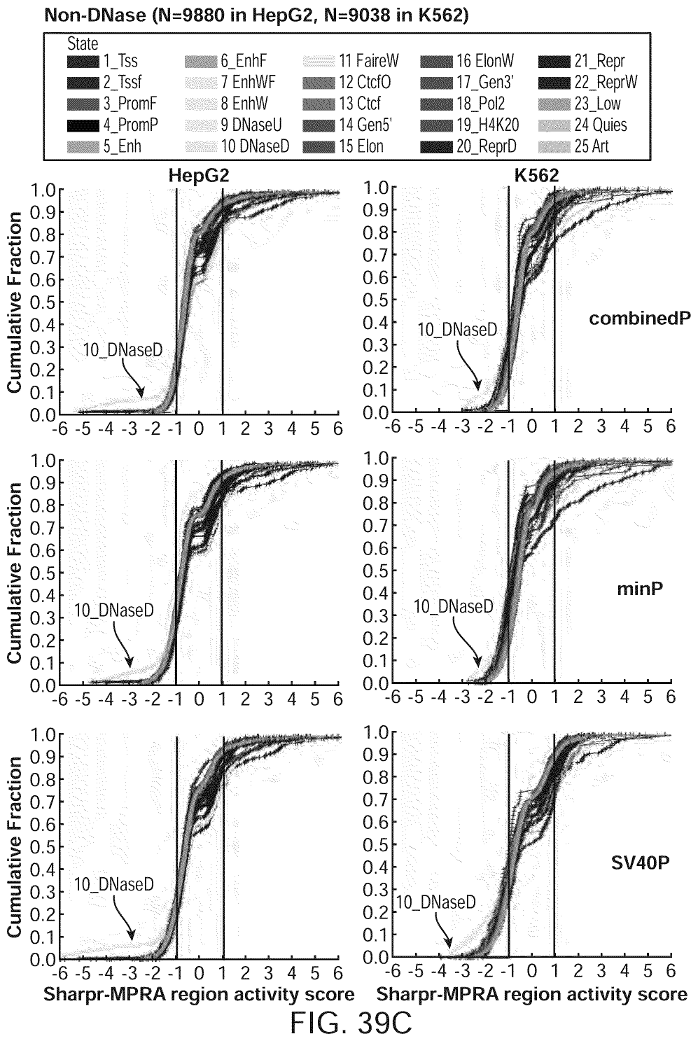

Beyond these thresholds, the full distribution of MaxPos scores across chromatin states and DNA accessibility (FIG. 39) confirmed consistently higher activation scores for active promoter and H3K27ac-marked enhancer states across a broad range of ranked positions. For non-DNase sites, the strongest repressive scores for chromatin state “DNaseD” (associated with single-cut DNase and lack of double-cut DNase) are found, indicating the inclusion of repressive elements (FIG. 39c).

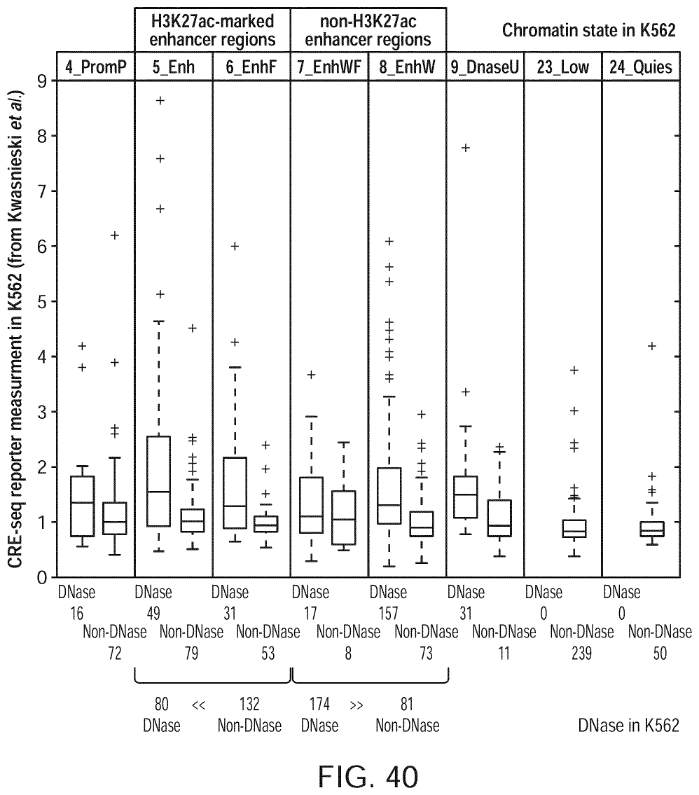

The role of H3K27ac as a signature of active enhancer regions is established and is in agreement with the results here, but has been questioned in another study using a reporter assay (CRE-seq), which indicated that H3K27ac-marked regions show weaker reporter activity. That study used a 7-state segmentation that merged ChromHMM and Segway results, and tested smaller segments (130 bp) without a tiling approach and without anchoring on DNase sites, making the results dependent on positioning of the tested segments, and specifically whether DNase sites or their flanking elements were captured. Mapping their tested segments on the 25-state ChromHMM annotations considered here, it is found that the H3K27ac-marked enhancers selected in the study preferentially were outside DNase sites, and the non-H3K27ac enhancers selected preferentially were in DNase sites. Correcting for this bias by analyzing DNase and non-DNase sites separately, it is found that H3K27ac enhancers had increased CRE-seq activity compared with non-H3K27ac enhancers (FIG. 40).

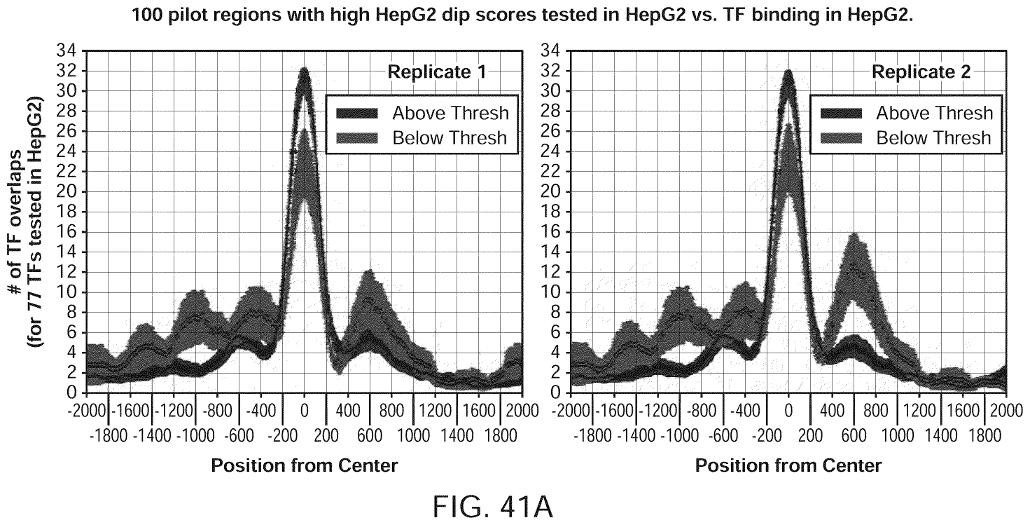

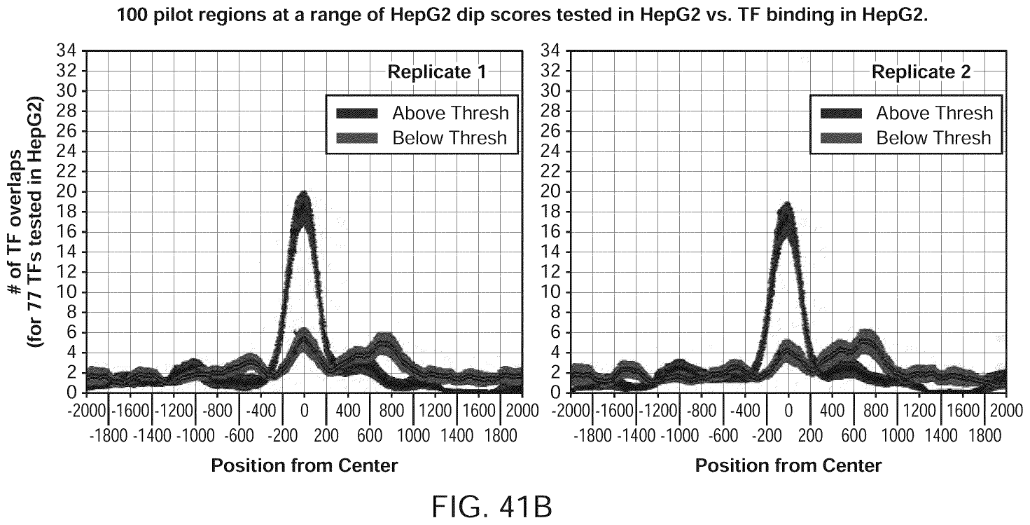

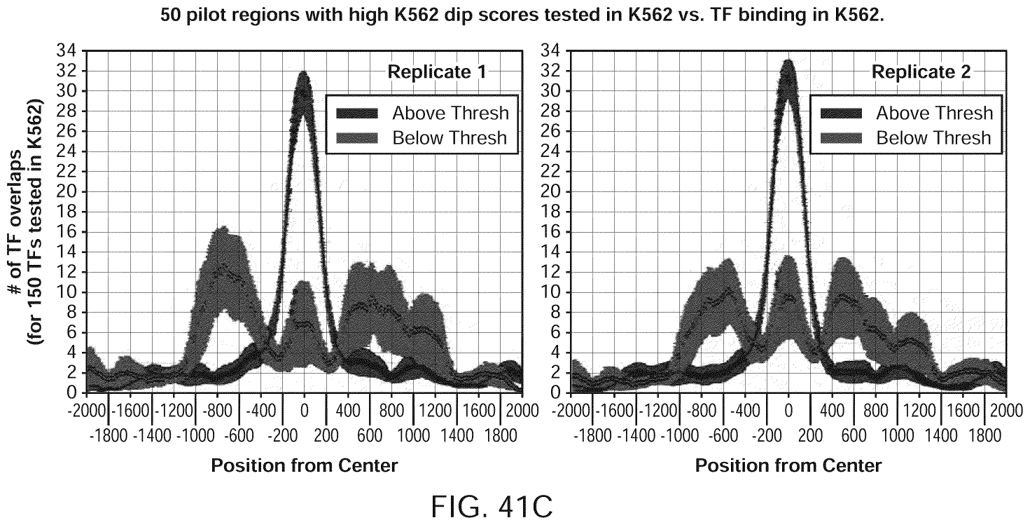

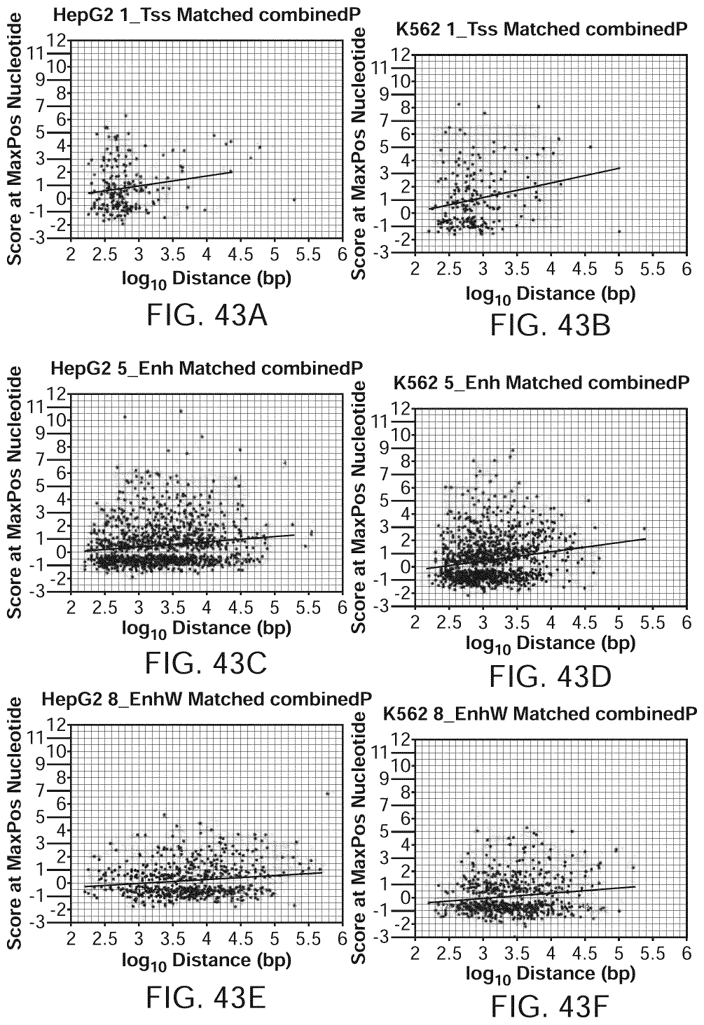

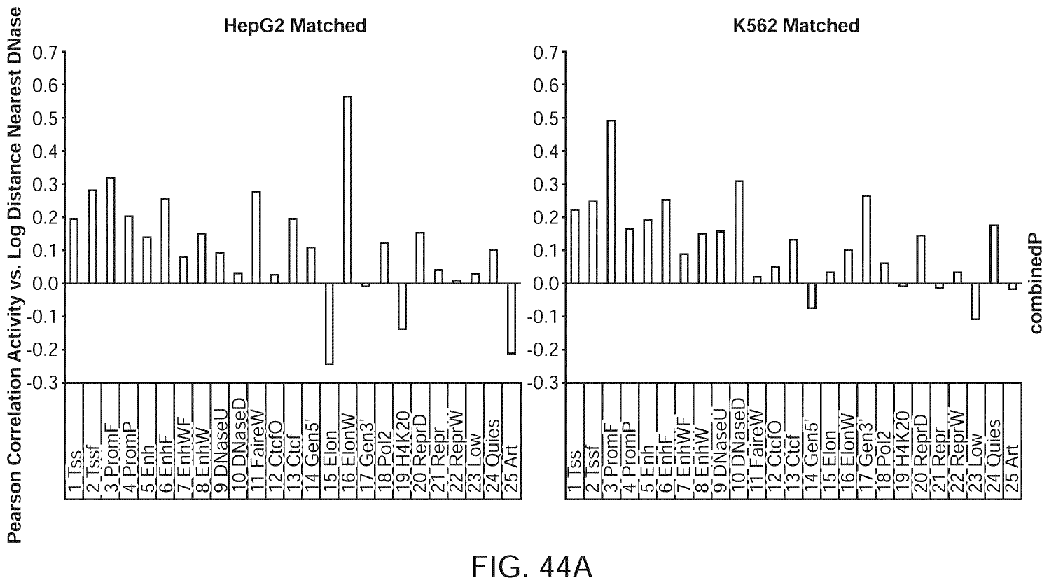

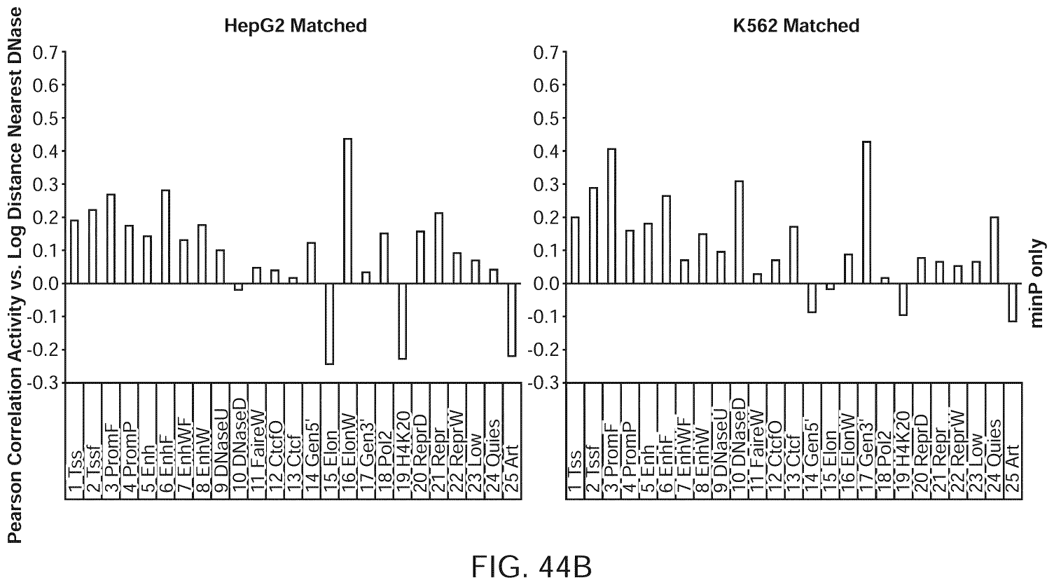

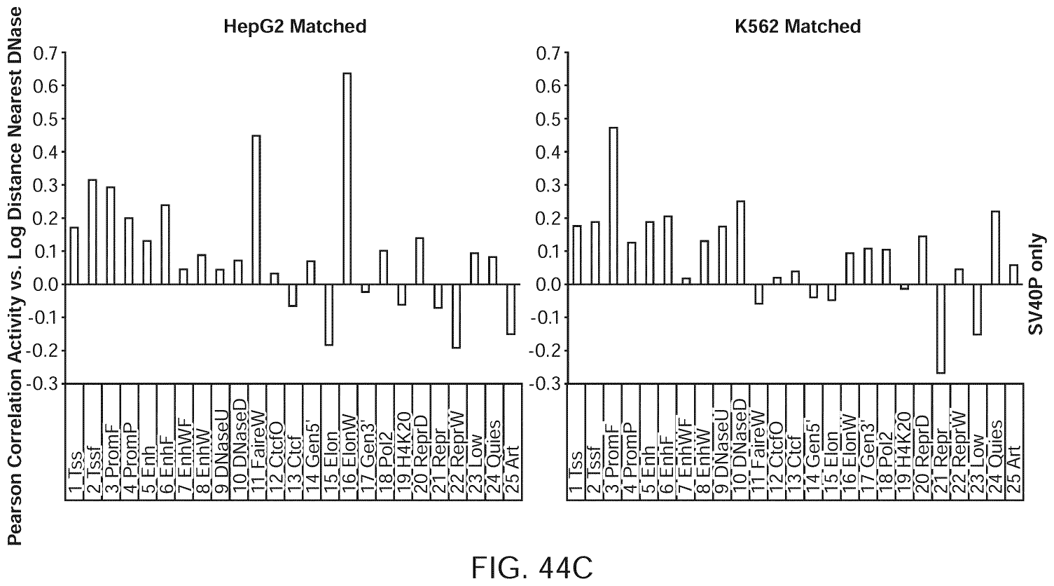

Many predicted enhancer and promoter regions did not have reporter activity (FIG. 13b). These regions showed distinct levels and patterns of endogenous TF binding and DNA accessibility, including less frequent endogenous TF binding in the tested regions, more frequent endogenous TF binding in the surrounding 2 kilobases, and proximity to other DNase sites (FIGS. 41-44). These findings are interpreted to indicate that their endogenous activating signatures may arise at least in part from TF binding in nearby regions, consistent with their lower reporter gene expression when tested in isolation.

A Subset of Repressive Motifs in Active Chromatin

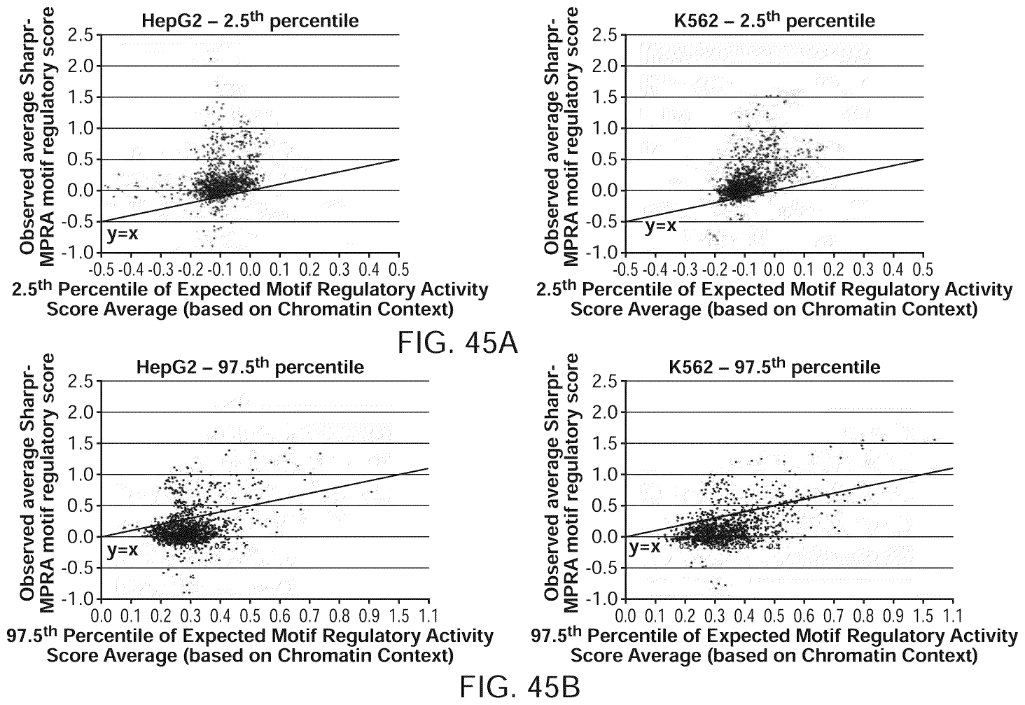

For each motif, the relationship between its observed average regulatory score and the average regulatory score that would be expected based on the chromatin states where the motif occurs is analyzed, quantified as the median of randomized motif occurrences that preserve positional and chromatin-state distributions (Methods). Overall, the observed average score of a motif correlated with its expected average score (about 0.54 in HepG2 cells and about 0.68 in K562 cells for motifs with ≥20 instances; FIG. 6c, FIG. 45). For example, NRF1 showed both a high average regulation score and a high expected score in both HepG2 and K562 cells, indicating that it acts as an activator in active chromatin states.

Several motifs showed moderate expected scores but very strong activating or repressive motif scores, indicating they maintain their functions regardless of their genomic context. In HepG2 cells, for example, TP53 showed a moderate expected score, but the highest score among all evaluated motifs, consistent with its proposed role as a pioneer factor. At the other end of the spectrum, REST showed an intermediate expected score but had the most repressive motif score, indicating strong repressor functions irrespective of context.

A small number of motifs showed repressive motif scores, but among the highest expected scores, indicating they play “attenuator” repressive roles in activating chromatin contexts. RFX family motifs had among the most repressive motif scores in HepG2, but among the most activating expected scores (FIG. 6c). Consistent with ‘attenuator’ roles, they showed repressive (negative) activity in the positional activity analysis, but they were flanked by activating (positive) scores (FIG. 4b). Moreover, in the pilot analysis in HepG2 cells, RFX family motifs were identified as enriched in segments inferred to be repressive, but were found in active enhancer regions (FIG. 10). Indeed, a repressive role has been experimentally confirmed in an enhancer of the CDX2 gene for a single RFX1 motif instance in HepG2 cells. The results indicate a broader repressive role in active regions, a discovery that stems directly from the ability to distinguish activating versus repressive nucleotides using the tiling approach.

### Conclusions

Disclosed herein is Sharpr-MPRA, a combined experimental and computational approach for high-resolution mapping of activating and repressive nucleotides across thousands of genomic regions. Dense tiling of MPRA constructs are used spanning about 4.6 million nucleotides targeting 15,720 regions at a resolution typically not afforded without perturbation experiments, which are typically not applicable at this scale. Sharpr-MPRA distinguishes activating from repressive nucleotides, and directly assesses regulatory function in a reporter assay, thus complementing endogenous epigenomic signatures surveyed by other methods.

Nucleotides with stronger activating or repressive Sharpr-MPRA scores were enriched in evolutionarily conserved elements and predicted cell-type-specific TF binding sites. Both enrichments were weaker for the most highly active nucleotides in HepG2 cells. Instead, the strongest reporter activity overlapped ERV1 endogenous retroviral repeat elements, which might indicate a potential role in regulatory turnover across even closely related species.

Endogenous epigenomic signatures were predictive of reporter gene expression, with chromatin state and DNA accessibility each providing relevant information. Segments with endogenous active promoter and H3K27ac-marked enhancer signatures drove the strongest average reporter gene activation, and segments showing endogenous Polycomb-associated signatures were among those with the strongest average reporter gene repression. These results indicate that even when tested outside their endogenous context, DNA sequence elements maintain the activating and repressive functions reflected in their endogenous epigenomic signatures.

Aggregation of activity scores at the motif level revealed cell-type-specific motifs, and distinguished activator and repressor motifs. Motif activity typically correlated with chromatin context, with activating motifs found in active chromatin states, and repressive motifs in repressive states. Notable exceptions included putative ‘attenuator’ motifs that showed repressive roles but were found in active chromatin states (for example, RFX motifs in HepG2 cells) and putative “pioneer” motifs, which showed strong activity regardless of their chromatin-state context (for example, activator TP53 and repressor NRSF), although directed experimentation and endogenous modulation can be used to confirm these predictions. Most motifs showed activating-only or repressive-only signatures, but a small number of “dual-role” motifs showed both activating and repressive signatures (for example, members of the MAF and AP-2 protein families). The sequence pattern with the strongest average activity in both cell types, TCTCGCGAGA, was not associated with a well-established regulator, highlighting the continued understanding of regulatory elements, and the importance of unbiased dissection of regulatory regions.

Conditions include the use of longer sequences to show reporter activity in some regions, which might be overcome by improved DNA synthesis, and constrained transfection efficiency in some cell types, which may specify alternative delivery approaches (for example, viral transduction). Additionally, additive effects are assumed in the analysis, and additional implementations can account for interactions between different nucleotide positions. Barcode effects and other factors may cause experimental noise, which can be overcome by higher density tiling or different experimental barcoding strategies. Tested are elements in episomal assays, which provide direct information on regulatory activity, but may not fully capture potential effects of the endogenous chromatin context, and unstimulated cells are transfected, although some sites may function after specific stimulations.

Diverse applications are envisioned for the results and methodology disclosed herein. For example, the annotation of activating or repressive function for about 4.6 million nucleotides can be used to probe biological questions beyond the ones addressed here, and to train computational models (for example, for predicting activating and repressive nucleotides outside the regions surveyed here, or predicting the effect of noncoding variation on regulatory function). As another example, Sharpr-MPRA's combined experimental and computational strategy can be useful for dissecting regulatory regions across individuals, species, cell types, conditions and disease states.

Methods

Pilot Large-Step Massively Parallel Reporter Assay Design.

As a Pilot Design, 250 regulatory regions are selected for testing. Each selected regulatory region was tiled by nine sequence tiles of 145 bp in length placed in 30-bp offsets so that adjacent sequences have 115 bp in common. Each tile was associated with 24 unique barcodes. In this design 216 barcodes per putative regulatory region is used.

Of the 250 regions, 200 are selected so that their center positions came from a HepG2 H3K27ac-marked “strong enhancer” chromatin state from the 15-state chromatin state model (FIG. 7b). To specify the locations to test, first a dip score is specified based on the ENCODE hg18 HepG2 H3K27ac signal on chromosomes 1-22 and chromosome X. It is specified that the dip score is to be the sum of the signal from positions 200 bp away in both directions minus twice the signal at the dip center. Then all positions are ranked at a 25-nucleotide resolution based on their dip score excluding from consideration positions that either (i) did not have the minimum HepG2 H3K27ac signal within 200 nucleotides, or (ii) were tied for the minimum H3K27ac signal with another position within 200 nucleotides and did not have a strictly greater dip score than the tied position. This condition often allowed centering the tiles in nucleosome-depleted regions. Positions are excluded if they were within 2 kb of an annotated transcription start site based on the GENCODE v2b annotations. Also excluded from testing are those sequences that contained GGTACC, TCTAGA or GGCCNNNNNGGCC, as these were recognition sequences of the restriction enzymes. 100 positions are selected to be the highest ranked non-excluded positions. An additional 100 positions are selected to cover a range of dip scores among the non-excluded positions, grouping the remaining regions (after the top 100) into 100 dip score ranges, and selecting the region with the maximal dip score in each range. Formally, let m denote the dip score of the 101^{st} ranked position. An interval width w is specified to be (ln(m)−ln(10))/99. For selections i=1, . . . , 100 made to cover a range of dip scores, as the i^{th} range is selected to be the region with the greatest dip score, v, such that it was still the case that ln(v)≤m−(i−1)×w. The center of the selected 25-bp position interval was used as the center of the center tile.

An additional 50 sequences were selected based on the same procedure to select the top 100 sequences for HepG2, but based on the K562 data, constrained to the top 50, with the additional constraint that they were in low-activity states in HepG2 (“weak transcribed” or “heterochromatin;low signal”) (FIG. 7b).

For the motif enrichment analysis, the coordinates are converted from this design to hg19 using the UCSC Genome Browser liftover tool.

Identification of Significant Adjacent Changes in the Pilot Large-Step Data.

Among all pairs of adjacent tiles, identified is a set of pairs that had a significant difference in reporter expression level at a 5% false discovery rate. The procedure for doing this was as follows. pᵢ denotes the P value for the i^{th} adjacent pair (where i=1, . . . , 2,000 in this case) that there is a significant difference in the reporter expression between the first and second tiles of the pair. p_{i,L} denotes the P value for the one sided test that the second tile has lower expression than the first and p_{i,G} the P value for the one-sided test that the second tile has greater expression than the first. p_{i,L,r} and p_{i,G,r} for r=1, R, where R=2 in this case, denote the P value for that in the i^{th} pair and the r^{th} replicate that the second tile had lower or greater expression than the first, respectively.

The individual and p_{i,L,r} and p_{i,G,r} P values are computed based on the one-sided Mann-Whitney test on all the individual barcoded expression values for a tile, which was up to 24 in this case. To compute p_{i,L,r}, first a two-sided P value, v, is obtained for the Mann-Whitney test using Apache Commons Math 3.3. The P value v/2 is assigned if the second tile had average lower or equal ranks than the first and the P value 1−v/2 otherwise; the opposite assignments are made for p_{i,G,r}. The p_{i,L} P value was computed based on Fisher's method combining the p_{i,L,r} for r=1, . . . , R P values, and likewise for the p_{i,G} P values. The P value pᵢ is specified to be pᵢ=min(1,2×min(p_{i,L}, p_{i,G})). Here, it is multiplied by two to correct for having tested both P values separately as one-sided tests. A false discovery rate is obtained for the set of P values pᵢ for i=1, . . . , 2,000 using a Benjamini-Hochberg procedure.

Scale-Up Sharpr-MPRA Assay Design.

15,720 DNase sites are targeted, including 3,930 tiled regions based on each of four cell types: HepG2, H1-hESC, K562 and HUVECs. The DNase sites were generated by the University of Washington ENCODE Group and specifically used location of the peak calls contained in the hg19 files: wgEncodeUwDnaseHepg2PkRep1.narrowPeak.gz, wgEncodeUwDnaseH1hescPkRep1. narrowPeak.gz, wgEncodeUwDnaseK562PkRep1.narrowPeak.gz, and wgEncode UwDnaseHuvecPkRep1.narrowPeak.gz for the HepG2, H1-hESC, K562 and HUVEC cell types, respectively. The selection of the subset of 3,930 regulatory regions based on each cell type was conducted independently. To increase chromatin state diversity, regions are selected using a richer 25-state chromatin state model (e.g., ChromHMM model), which was based on 14 input tracks, including 8 histone modification marks, CTCF, POL2, DNase (single-cut, generated by Duke; and double-cut, generated by University of Washington), FAIRE and input. The counts of each state were manually specified to ensure some coverage of each chromatin state, greater coverage in states more associated with DNase, and deeper coverage of enhancer chromatin states (FIG. 1b). The regions were then randomly selected given the counts for each state. As with the Pilot design, excluded from testing are those sequences that contained GGTACC, TCTAGA or GGCCNNNNNGGCC as recognition sequences of the restriction enzymes, but with no restriction in this design with respect to position relative to annotated genes. Selected regions were tiled with 31 sequence tiles each 145-bp long and placed at 5-bp offsets, centered on the DNase site. Each tile was associated with a single barcode. The tiled regions based on each cell type and each chromatin state were randomly and evenly divided between the two array designs, which led to each design having 7,860 tiled regions. If the same or overlapping regions were selected based on different cell types, both tiled regions are retained and considered separately except in forming the browser tracks in which case all regulatory scores are averaged for a given nucleotide. In total, 15,720 regions are targeted, some of which overlapped, resulting in 15,455 unique non-overlapping regions.

Experimental procedure. Certain experimental methods for a massively Parallel Reporter Assay are Performed as Described in Melnikov et al., Massively Parallel reporter assays in cultured mammalian cells. J. Vis. Exp. 90, 90, e51719 (2014). Oligonucleotide library synthesis was performed, and the cell culture, transfection and plasmid construction are performed as described in Kheradpour et al., Systematic dissection of regulatory motifs in 2000 predicted human enhancers using a massively parallel reporter assay. Genome Res. 23, 800-811 (2013). The MPRA vector backbone and promoter-reporter cassettes are available from Addgene (plasmids 49349, 49353 and 49354). The K562 and HepG2 cell lines were obtained directly from ATCC (CCL-243 and HB-8065). ATCC performed routine authentication using STR analysis. Separate authentication or mycoplasma testing are not performed.

For the pilot design, a single 55K-spot array is used for synthesis and sequences are designed for 54,000 of them (2,250 unique sequences with 24 barcodes each). Transfection and barcode sequencing experiments were conducted in replicate in both K562 and HepG2 cells using the SV40 promoter (FIG. 7a). The plasmid pools were amplified and sequenced in replicate and shared among the K562 and HepG2 experiments.

For the scale-up design, two 244K-spot arrays are used for synthesis and sequences are designed for 243,660 of them, of which 243,573 and 243,564 (about 99.96%) were included in the synthesis for the two arrays, leading to a total of 487,137 probed tiles. Transfection and barcode sequencing experiments were conducted for each array in both HepG2 and K562, each using both a minimal and SV40 promoter, each in two replicates (16 experiments total). For these experiments four plasmid DNA pools are amplified and sequenced, one for each combination of array design and promoter type, with RNA replicate experiments normalized to the same plasmid DNA pool.

Data Normalization.

The initial data processing for data from one experiment was as follows. DNA and RNA counts are generated based on the procedure described in Kheradpour et al., and a pseudocount of 1 is added to these counts for smoothing. Then, all RNA values are divided by the sum of all RNA values, and the DNA values are divided by the sum of all the DNA values. For the readout corresponding to a given barcode the log base two ratio of the RNA to DNA counts are computed. Those barcodes that had less than 20 for the original DNA counts associated with them are treated as missing. For the pilot design, the median value among multiple barcodes of the same sequence tile is used. Then, these values are normalized by taking the difference with the average value for the expression of the tiles in the positions furthest from the H3K27ac dip centers (tiles #1 and #9) as an approximation for background in these experiments. For the scale-up design, additional normalization was conducted on the inferred activity values (see below).

Sharpr-MPRA Regulatory Activity Scores.

To compute Sharpr-MPRA regulatory activity scores from tiled reporter data, it is assumed that each reporter sequence had length L, which was 145 bp in this case, and a consistent step size, s, between adjacent reporter sequences, which was 5 in this case. It is assumed that L was divisible by s, and let N=L/s denote the number of intervals of the step size overlapped by a reporter sequence, which was 29 in this case. J denotes the number of overlapping reporter tiles covering a tiled region, which was 31 in this case. K denotes the total number of non-overlapping intervals of step size s intervals that have coverage by at least one reporter sequence which is N+(J−1), or 59 in the case. W=s×K denotes the total number of nucleotides covered by at least one reporter sequence, which was 295 in the case, and they are indexed using i=1, . . . , 295. T denotes the total number of tiled regions tested in a single design, which was 7,860 in the case. R denotes the number of experiments for the design that are been combined, where R=2 when the SV40P and minP experiments are separately considered, and R=4 when all experiments for a design in the same cell type are combined.

It is assumed that A_{r,t,k} denotes a random variable for the unobserved regulatory activity for the k^{th} s=5-bp interval where k=1, . . . , K=59 in the t^{th} tiled region in the r^{th} experiment being combined. It is assumed that M_{r,t,j} denotes a random variable for the normalized expression value for the reporter sequence at the j^{th} tile offset, where j=1, . . . , J=31, of the t^{th} tiled region in the r^{th} experiment being combined (FIG. 3a). m_{r,t,j} denotes the corresponding observed value, and if there was no observed value it is set to null. The objective is to infer the maximum a posteriori values of the A_{r,t,k} variables conditioned on the observed values for the M_{r,t,j} variables.

It is assumed that each A_{r,t,k} is normally distributed with mean μ_{α}_{ᵣ} and variance that is

A_{r,t,k}˜N(μ_{α}_{ᵣ},σ_{α}²) (1)

Let [symbol] ={m_{r,t,j}|m_{r,t,j}≠null} denote the multiset of all observed reporter values in the r^{th} experiment. μ_{α}_{ᵣ} is set to the empirical mean of the observed reporter values in the r^{th} experiment, that is,

$${μ}_{{a}_{r}} = \frac{1}{\vert \vert} {\sum }_{m ∈} m \qquad ( 2 )$$

σ_{α}² is a free parameter. Inferences are performed with σ_{α}² set both to 1 and 50, and the inferences are combined using a procedure described below.

It is assumed that each M_{r,t,j} is normally distributed with mean μₘ_{_{r,t,j}}, and variance σₘ_{ᵣ}², that is

$${M}_{r , t , j} ∼ N ( {μ}_{{m}_{r , t , j}} , {σ}_{{m}_{r}}^{2} ) \qquad ( 3 )$$

It is assumed μₘ_{_{r,t,j}} to be the mean of all the unobserved regulatory activity variables corresponding to intervals for which the j^{th} reporter tile overlaps, σₘ_{ᵣ}², is set to the empirical variance of the observed reporter values in r^{th} experiment, that is

$${μ}_{{m}_{r , t , j}} = \frac{1}{N} {\sum }_{l = 0}^{N - 1} {A}_{r , t , j + l} \\ {μ}_{{m}_{r}}^{2} = \frac{1}{\vert \vert} {\sum }_{m ∈} {( m - \frac{1}{\vert \vert} {\sum }_{m ∈} m )}^{2} \qquad ( 4 )$$

The vector X_{r,t}, comprised of the A_{r,t,k} and M_{r,t,j}, variables in the r^{th} experiment for t^{th} tiled region, can be expressed as a multivariate normal distribution as follows

$${X}_{r , t} = [ \begin{matrix}{A}_{r , t} \\ {M}_{r , t}\end{matrix} ] ∼ N ( {μ}_{{x}_{r , t}} , {Σ}_{{x}_{r , t}} ) \qquad ( 5 )$$

where A_{r,t}=[A_{r,t,1} . . . A_{r,t,K}]^{T} and M_{r,t}=[M_{r,t,1} . . . M_{r,t,I}]^{T}. If a m_{r,t,j} is considered missing, that is, has a null value, the corresponding M_{r,t,j} variable was omitted, the remaining M_{r,t,j} variables are not reindexed. And

$${μ}_{{x}_{r , t}} = [ \begin{matrix}{μ}_{{A}_{r , t}} \\ {μ}_{{M}_{r , t}}\end{matrix} ] , \\ {Σ}_{{x}_{r , t}} = [ \begin{matrix}{Σ}_{{A}_{r , t} , {A}_{r , t}} & {Σ}_{{A}_{r , t} , {M}_{r , t}} \\ {Σ}_{{M}_{r , t} , {A}_{r , t}} & {Σ}_{{M}_{r , t} , {M}_{r , t}}\end{matrix} ] \qquad ( 6 )$$

Both μ_{A}_{_{r,t}} and μ_{M}_{_{r,t}} are column vectors with all values equal to μ_{α}_{ₜ}.

Conditioning on observing M_{r,t}=m_{r,t} the maximum a posteriori values for values in A_{r,t}, was determined as the mean in a conditional multivariate normal distribution which is given by

μ_{A}_{_{r,t}}+Σ_{A}_{_{r,t}}_{,M}_{_{r,t}}Σ_{M}_{_{r,t}}_{,M}_{_{r,t}}⁻¹(m_{r,t}−μ_{M}_{_{r,t}}) (7)

For Σ_{Mr,t,Mr,t}:

$${Σ}_{{M}_{r , t , u} , {M}_{r , t , u}} = {σ}_{{m}_{r}}^{2} + \frac{{σ}_{a}^{2}}{N} \\ {Σ}_{{M}_{r , t , u} , {M}_{r , t , v}} = \frac{{σ}_{a}^{2} ( N - \vert u - v \vert )}{{N}^{2}} if ( 0 < \vert u - v \vert < N ) \\ {Σ}_{{M}_{r , t , u} , {M}_{r , t , v}} = 0 if ( \vert u - v \vert \geq N ) \qquad ( 8 )$$

For Σ_{Ar,t,Mr,t}:

$${Σ}_{{A}_{r , t , k} , {M}_{r , t , u}} = \frac{{σ}_{a}^{2}}{N} if u \leq k < u + N \\ {Σ}_{{A}_{r , t , k} , {M}_{r , t , u}} = 0 otherwise \qquad ( 9 )$$

The above expressions are derived in Supplementary Note 1.

It is assumed that a_{r,t,k}, denotes the inferred value for the k^{th} interval in the t^{th} tiled region in the r^{th} experiment. It is noted that the modeling to infer these values can also be viewed as a specific instance of Bayesian linear regression.

Then, all inferred values are standardized within an experiment by subtracting the mean and dividing by the standard deviation to derive z_{r,t,k}, the standardized regulatory score for the k^{th} interval in the t^{th} tiled region in the r^{th} experiment (Supplementary Note 2).

\hat{z}_{t,k}′ is specified as the merged regulatory score for the k^{th} interval in the t^{th} tiled region, which averages the standardized regulatory score from multiple experiments (Supplementary Note 2).

When conducting inference with two different values for π_{α}², denoted σ_{α1}² and σ_{α2}², the merged regulatory scores are denoted for k^{th} interval in the t^{th} tiled region for these two parameter settings,

$${\hat{z}}_{t , k}^{{σ}_{{a}_{1}}^{2}} and {\hat{z}}_{t , k}^{{σ}_{{a}_{2}}^{2}} ,$$

respectively.

The scores are combined to obtain the value denoted \hat{z}_{t,k}′ with a procedure that takes the more conservative score when the signs of the values agree and 0 otherwise (Supplementary Note 2). Note that if σ_{α1}²=σ_{α2}², then

$${\hat{z}}_{t , k}^{′} = {\hat{z}}_{t , k}^{{σ}_{{a}_{1}}^{2}} = {\hat{z}}_{t , k}^{{σ}_{{a}_{2}}^{2}} .$$

Activity values are inferred using both a low-variance prior (σ_{α1}²=1) and a high-variance prior (σ_{α2}²=50). It is found that this strategy for combining the results using two different variance prior parameters was more robust compared to using one specific parameter setting as it would reduce overfitting in cases when a single variance parameter was set to be too large, which may lead to unlikely high activating and repressive inferences in the same small region, while also reducing underfitting when a single variance parameter was set to be too small (FIG. 12). The enrichments of inferred highly activating and repressive nucleotides for conserved elements are evaluated as a function of the variance prior parameters. It is found that they are relatively robust across a substantial range of parameter settings, and in particular when using this strategy of using the more conservative inference from two substantially different settings for the variance prior (FIG. 12c).

To obtain nucleotide regulatory activity scores, b_{t,i}, for each nucleotide i=0, . . . , W−1 in the t^{th} tiled region, piecewise linear interpolations between \hat{z}_{t,k}′ values (Supplementary Note 3) are conducted.

The inferences on the two designs were conducted separately, but they were combined for conducting the downstream analysis. The matrix operations were implemented using the library Apache Commons Math library v3.3. For the analysis on step sizes greater than 5 (FIGS. 28 and 29), the method is effectively applied with a step size of 5, but treated entire positions of reporter tiles as missing.

Region and Chromatin State Scores.

The region score for a tiled region t, denoted eₜ was specified as b_{t,i} (see above) where i is selected to maximize |b_{t,i}| for i=0, . . . , W−1. The average region score for a chromatin state u, denoted sᵤ, based on matched data in a cell type, is the average value of eₜ for all tiled regions t selected based on chromatin state u in the cell type.

Footprint, Motif, Transcription Factor ChIP-Seq, Conservation and Repeat Data.

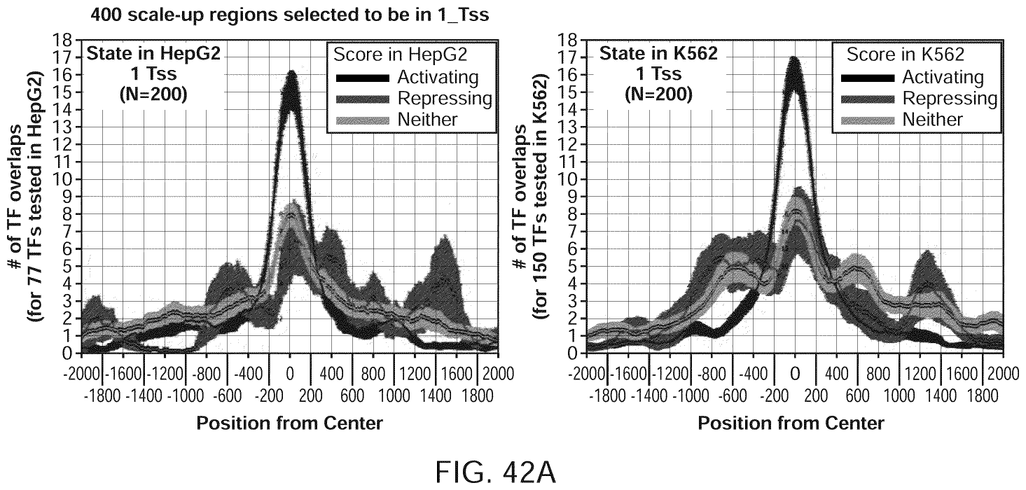

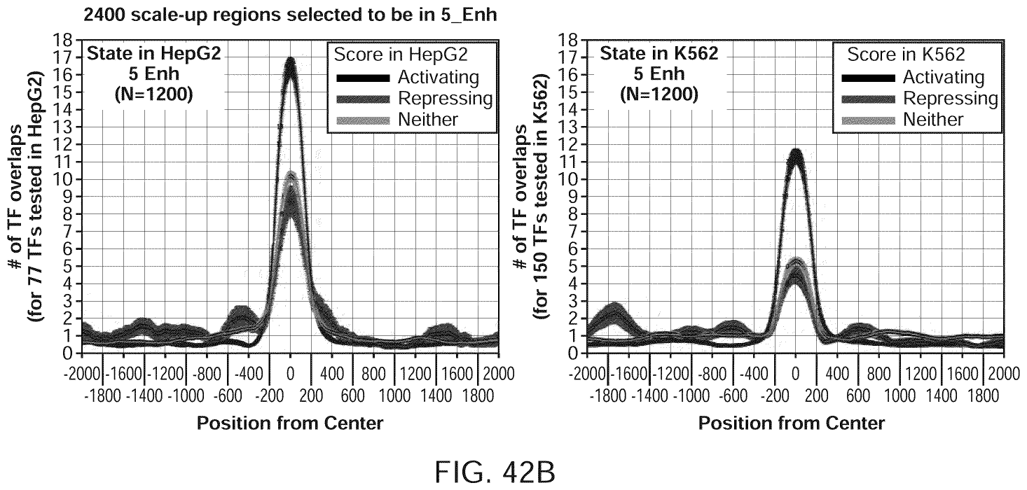

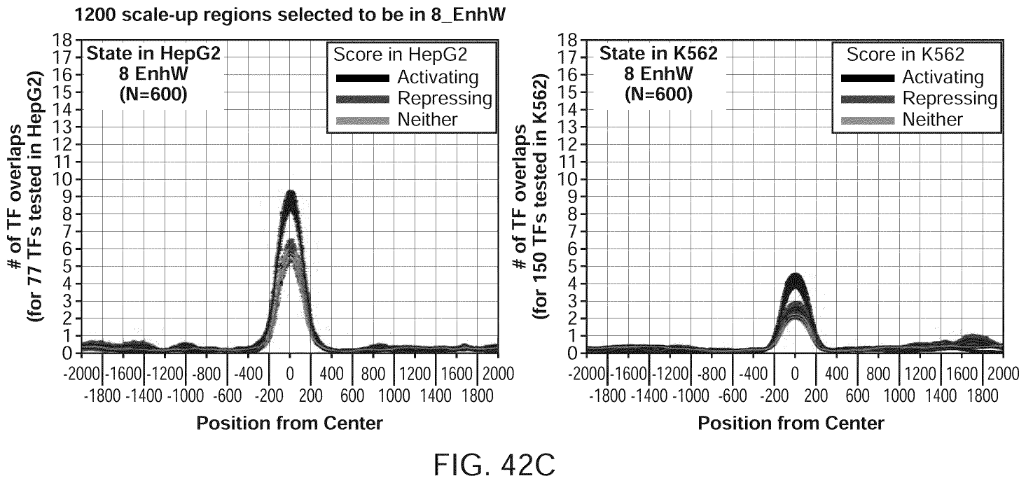

The HepG2 and K562 CENTIPEDE elements were obtained from http://centipede.uchicago.edu/. Certain footprints were obtained from http://ftp.ebi.ac.uk/pub/databases/ensembl/encode/supplementary/integration_data_jan2011/by DataType/footprints/jan2011. The Wellington footprints in K562 were obtained from Piper et al., Wellington: a novel method for the accurate identification of digital genomic footprints from DNase-seq data, Nucleic Acids Res. 41, e201 (2013). The hg19 footprints were obtained from http://fureylab.web.unc.edu/datasets/footprints/. The PIQ footprints were the version 1 footprints obtained from http://piq.csail.mit.edu/including both forward and reverse footprints. The motif instances were those provided by Kheradpour et al., Systematic discovery and characterization of regulatory motifs in ENCODE TF binding experiments, Nucleic Acids Res. 42, 2976-2987 (2014). The ENCODE transcription factor binding peak call data sets used in the analyses of FIGS. 41 and 42 were the ENCODE uniform peak calls downloaded from http://hgdownload.cse.ucsc.edu/goldenPath/hg19/encodeDCC/wgEncode AwgTfbsUniform, which included 150 files in K562 cells and 77 for HepG2 cells. The conserved elements were the hg19 liftover of the SiPhy-PI conserved elements from Lindblad-Toh et al., A high-resolution map of human evolutionary constraint using 29 mammals, Nature 478, 476-482 (2011). The repeats were based on RepeatMasker obtained through the UCSC genome browser.

The Motif Analysis in the Scale-Up Experiments Data.

The motif analysis comparing K562 and HepG2 cells (FIG. 4, FIG. 31) was computed based on averaging the b_{t,i} values at the center of all instances for a motif. The P values were computed using a paired t-test over all instances tested using the Apache Commons Math library v3.3 implementation. The average motif score for the analysis in FIG. 6c was based on motif instances overlapping a tiled region selected based on HepG2 chromatin data when testing in HepG2 cells and likewise for K562 restricted to motifs with at least 20 such instances. The expected motif scores for these same set of instances were computed by permuting among tiled regions assigned to the same chromatin state and selected by the cell type of the measurements, and which set of reporter values were assigned to which tiled regions. These permutations would preserve the same set of rows in a matrix where the rows correspond to reporter expression values and the columns the tile offsets. This was done for 1,000 permutations. For each motif the median average motif score across all permutations as well as the value of the 2.5% and 97.5% quantiles were recorded to form the expected motif values and 95% confidence intervals.

The P values for motif enrichment as an activator or repressor in FIG. 4c and FIGS. 31, 32 and 35 were computed based on one-sided binomial tests where the probability of success in the binomial distribution is the fraction of total nucleotides tested that had a regulatory score greater than or equal to the activation threshold for activators or less than or equal to the repression threshold for repressors. The number of trials is the number of instances of a motif with a center position overlapping a nucleotide tested. The number of successes is the number of instances of the motif with a center position having a regulatory score equal to or greater than the activation threshold for activators or less than or equal to the repression threshold for repressors. The P value threshold for specifying activator and repressor motifs was an uncorrected P value of 0.01. In total, 1,934 motifs were tested. Motif instances that appeared on both strands at the same position were counted once in the analysis.

Motif Analysis in the Pilot Large-Step Experiment Data.

Four sets of sequences are specified to analyze for motifs based on the pilot data. Two sets were specified based on adjacent pairs of tiles with significant differences at a 5% FDR in the HepG2 data, with one set corresponding to the sequences that on average had higher expression as determined based on the average ranks and the other set had lower expression. The other two sets were based on the K562 data specified in the same way as for the HepG2 data. For each set of sequences motif analysis is conducted on the 30 bp that were unique to each sequence in the set compared to its corresponding adjacent tile plus ten additional base pairs into the common sequence. The motif enrichments with known motifs were computed using the program of Kheradpour et al., Systematic discovery and characterization of regulatory motifs in ENCODE TF binding experiments, Nucleic Acids Res. 42, 2976-2987 (2014), modified so that the background set of motifs solely included those overlapping sequences that are part of the array design. De novo motif discovery is run using MEME through the MEME suite with its default settings except requesting 10 motifs. The motifs were matched to an expected motif using TOMTOM.

DeltaSVM Comparison.

For the comparison with regulatory mutations predicted by the DeltaSVM approach in FIG. 24a, a top 1% set of nucleotides associated with the maximum decrease in the sequence predicted to be regulatory when mutating the reference sequence is identified. Specifically, the gkm-SVM 10-mer weights are obtained based on human ENCODE UW DHS from the website: http://www.beerlab.org/deltasvm/and used those in the files tup2_UwDnaseHepg2Aln_500_nc30_np_top10k_nsr1×1_gkm_1_10_6_3_weights.out and tup2_UwDnaseK562Aln_500_nc30_np_top10k_nsr1×1_gkm_1_10_6_3_weights. out for Hepg2 and K562 cells, respectively. For each nucleotide tested, the sum of the k-mer weights is summed for the k-mers overlapping the nucleotide, which would be ten weights or fewer if the nucleotide was within the first or last nine nucleotides of the 295-bp region. This sum is denoted as S_{REF}. This sum is computed for each of the three possible nucleotide substitutions to the reference sequence at the position denoted by s_{M1}, s_{M2} and s_{M3}. Nucleotide position is ranked based on the extent to which they minimized min(s_{M1}-s_{REF}, s_{M2}-s_{REF}, s_{M3}-s_{REF}). The focus on the top 1% nucleotides was consistent with a percentage threshold used previously with DeltaSVM scores.

A top 1% set of nucleotides is also identified associated with the maximum increase in the sequence predicted to be regulatory when mutating the reference sequence (FIG. 24b) using the same procedure as above except ranking nucleotides based on the extent to which they maximized the value of max(s_{M1}-s_{REF}, s_{M2}-s_{REF}, s_{M3}-s_{REF}).

Barcode k-Mer Analysis.

For the analysis of barcode k-mer effect on inferred activity (FIG. 18), e_{t,j} is first specified to be the barcode sequence for the t^{th} tiled region where t=1, . . . , 15,720, for the j^{th} reporter tile offset where j=1, . . . , 31, and e_{t,j,p} the nucleotide at the p^{th} position in the barcode for p=1, . . . , 10.

For considering the occurrence of a k-mer sequence regardless of position within the barcode, then for a k-mer sequence s the set U_{s,j}={(t,p)|s=e_{t,j,p} . . . e_{t,j,p+k-1}} is specified, which gives all pairs of regions and barcode positions containing the k-mer sequence in the j^{th} reporter tile offset. Average inferred regulatory activity scores are computed for all tuples (s, j, i) such that s is a sequence of length k found within at least one barcode sequence, j=1, . . . , 31 corresponds to one of the 31 reporter tile offsets, and i=1, . . . , 295 corresponds to one of the inferred activity positions. The average inferred regulatory activity average is then specified as

$$\frac{1}{\vert {U}_{s , j} \vert} {\sum }_{u ∈ {U}_{s , j}} {b}_{u . t , i} \qquad ( 10 )$$

where u.t denotes the tiled region of u. These averages are then ranked to determine the cumulative distributions. To determine the expected distribution of these averages for this analysis, each barcode sequence is randomly assigned to reporter sequences, but still preserving the activity inferences based on the actual assignments. The same analysis is repeated based on the randomized assignments. This is done for 400 randomizations of barcode assignments to reporter sequences to obtain 400 separate rankings of averages. For each rank in the ranking, the median value over the 400 randomizations and the 2.5 and 97.5^{th} percentiles are determined. This analysis was done separately for each value of k=1, . . . , 6.

Supplementary Note 1—Derivation of Σ_{Mr,t,Mr,t} and Σ_{Ar,t,Mr,t} equations

For Σ_{Mr,t,Mr,t}:

$${\sum }_{{M}_{r , t , u} , {M}_{r , t , u}} = {σ}_{{m}_{r}}^{2} + {σ}_{a}^{2} / N$$

$${\sum }_{{M}_{r , t , u} , {M}_{r , t , v }} = \frac{{σ}_{a}^{2} ( N - \vert u - v \vert )}{{N}^{2}} if ( 0 < \vert u - v \vert < N )$$

$${\sum }_{{M}_{r , t , u} , {M}_{r , t , v}} = 0 if ( \vert u - v \vert \geq N )$$

The expression for Σ_{Mr,t,u,Mr,t,u} above follows from observing:

$$\begin{matrix}var ( {M}_{r , t , u} ) = var ( {σ}_{{m}_{r}} {Z}_{r , t , u} + ( \frac{1}{N} {\sum }_{i = 0}^{N - 1} {A}_{r , t , u + i} ) ) \\ = {σ}_{{m}_{r}}^{2} + \frac{1}{{N}^{2}} {\sum }_{l = 0}^{N - 1} var ( {A}_{r , t , u + l} ) = {σ}_{{m}_{r}}^{2} + {σ}_{a}^{2} / N\end{matrix}$$

where Z_{r,t,u} represents a standard normal random variable.

The expression for Σ_{Mr,t,u,Mr,t,v} follows since

$$\begin{matrix}cov ( {M}_{r , t , u} , {M}_{r , t , v} ) = cov ( {σ}_{{m}_{r}} {Z}_{r , t , u} + \frac{1}{N} {\sum }_{l = 0}^{N - 1} {A}_{r , t , u + l} , {σ}_{{m}_{r}} {Z}_{r , t , v} + \frac{1}{N} {\sum }_{l = 0}^{N - 1} {A}_{r , t , v + l} ) \\ = cov ( \frac{1}{N} {\sum }_{l = 0}^{N - 1} {A}_{r , t , u + l} , \frac{1}{N} {\sum }_{l = 0}^{N - 1} {A}_{r , t , v + l} )\end{matrix}$$

where Z_{r,t,u} and Z_{r,t,v} represent independent standard normal random variables. The expression is 0 if (|u−v|≥N) and otherwise is

$$\frac{1}{{N}^{2}} {{\sum }^{N - 1}}_{l = \vert u - v \vert} var ( {A}_{r , t , ( m i n ( u , v ) + l )} ) = \frac{{σ}_{a}^{2} ( N - \vert u - v \vert )}{{N}^{2}}$$

For Σ_{Ar,t,Mr,t}:

$${\sum }_{{A}_{r , t , k} , {M}_{r , t , u}} = \frac{{σ}_{a}^{2}}{N} if u \leq k < u + N$$

$${\sum }_{{A}_{r , t , k} , {M}_{r , t , u}} = 0 otherwise$$

This follows from observing:

$$\begin{matrix}cov ( {A}_{r , t , k} , {M}_{r , t , u} ) = cov ( {A}_{r , t , k} , \frac{1}{N} {\sum }_{l = 0}^{N - 1} {A}_{r , t , u + l} ) \\ = \frac{1}{N} {\sum }_{l = 0}^{N - 1} cov ( {A}_{r , t , k} , {A}_{r , t , u + l} )\end{matrix}$$

All terms are 0 except if u≤k<u+N in which case the expression reduces to

$$= \frac{1}{N} var ( {A}_{r , t , k} )$$

Supplementary Note 2—Combining Inferences from Multiple Experiments and Priors

This section provides equations for the procedure described in the Methods that translate activity inferences based on each experiment and prior variance separately to one that provides a single combined score.

First, all inferred values are standardized within an experiment by subtracting the mean and dividing by the standard deviation. Formally z_{r,t,k} denotes the standardized regulatory score for the k^{th} interval in the t^{th} tiled region in the r^{th} experiment and where:

$${μ}_{{\hat{A}}_{r}} = \frac{1}{\vert T \times K \vert} {\sum }_{t = 1}^{T} {\sum }_{k = 1}^{K} {a}_{r , t , k}$$

$${σ}_{{\hat{A}}_{r}} = \sqrt{\frac{1}{\vert T \times K \vert} {\sum }_{t = 1}^{T} {\sum }_{k = 1}^{K} {( {a}_{r , t , k} - \frac{1}{\vert T \times K \vert} {\sum }_{t = 1}^{T} {\sum }_{k = 1}^{K} {a}_{r , t , k} )}^{2}}$$

$${z}_{r , t , k} = \frac{( {a}_{r , t , k} - {μ}_{{\hat{A}}_{r}} )}{{σ}_{{\hat{A}}_{r}} }$$

Also specified is the merged regulatory score \hat{z}_{t,k} for the k^{th} interval in the t^{th} tiled region, which averages multiple replicates as:

$${\hat{z}}_{t , k} = \frac{1}{R} {\sum }_{r = 1}^{R} {z}_{r , t , k}$$

When conducting inference with two different values for σ_{α}², denoted σ_{α1}² and α_{α2}², the merged regulatory scores for k^{th} interval in the t^{th} tiled region for these two parameter settings are denoted,

$${\hat{z}}_{t , k}^{{′σ}_{a 1}^{2}} and {\hat{z}}_{t , k}^{{σ}_{a 2}^{2}} ,$$

respectively. These scores are combined to obtain the value denoted \hat{z}_{t,k}′ as follows

$${\hat{z}}_{t , k}^{′} = min ( {\hat{z}}_{t , k}^{{σ}_{{a}_{1}}^{2}} , {\hat{z}}_{t , k}^{{σ}_{{a}_{2}}^{2}} ) if {\hat{z}}_{t , k}^{{σ}_{{a}_{1}}^{2}} > 0 and {\hat{z}}_{t , k}^{{σ}_{{a}_{2}}^{2}} > 0$$

$${\hat{z}}_{t , k}^{′} = max ( {\hat{z}}_{t , k}^{{σ}_{{a}_{1}}^{2}} , {\hat{z}}_{t , k}^{{σ}_{{a}_{2}}^{2}} ) if {\hat{z}}_{t , k}^{{σ}_{{a}_{1}}^{2}} < 0 and {\hat{z}}_{t , k}^{{σ}_{{a}_{2}}^{2}} < 0$$

$${\hat{z}}_{t , k}^{′} = 0 otherwise$$

Supplementary Note 3—Interpolation to Nucleotide Scores

This section provides equations to derive the nucleotide regulatory activity scores, b_{t,i}, for each nucleotide i=0, . . . , W−1 in the t^{th} tiled region based on piecewise linear interpolation as presented in the Methods.

Formally b_{t,i} is specified as

$${b}_{t , i} = {\hat{z}}_{t , ⌊ \frac{i}{s} ⌋ + 1}^{′} if ( i < s / 2 ) or ( ( i mod s ) = ⌊ \frac{s}{2} ⌋ ) or ( i \geq W - \frac{s}{2} )$$

$${b}_{t , i} = ( 1 - g ) \times {\hat{z}}_{t , ⌊ \frac{i}{s} ⌋}^{′} + g \times {\hat{z}}_{t , ⌊ \frac{i}{s} ⌋ + 1}^{′} else if ( i mod s ) < ⌊ \frac{s}{2} ⌋$$

$${b}_{t , i} = ( 1 - h ) \times {\hat{z}}_{t , ⌊ \frac{i}{s} ⌋ + 1}^{′} + h \times {\hat{z}}_{t , ⌊ \frac{i}{s} ⌋ + 2}^{′} otherwise$$

$$where$$

$$g = ( ⌊ \frac{s}{2} ⌋ + ( i mod s ) + 1 ) / s$$

$$h = ( ( i mod s ) - ⌊ \frac{s}{2} ⌋ ) / s$$

### Computing Device

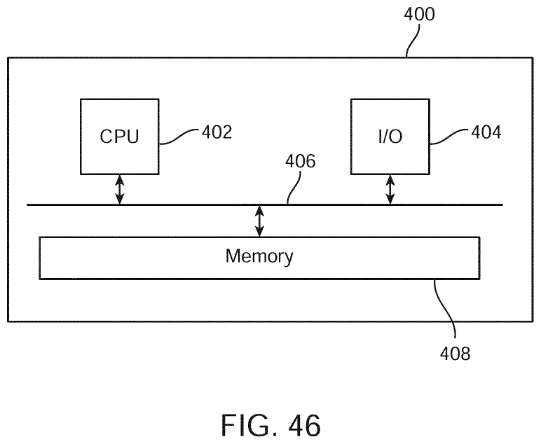

FIG. 46 shows a computing device 400 implemented in accordance with some embodiments of this disclosure. Referring to FIG. 46, the computing device 400 includes a processor 402 (e.g., a central processing unit (CPU)) that is connected to a bus 406. Input/output (I/O) devices 404 are also connected to the bus 406, and can include a keyboard, mouse, display, and the like. An executable program, which includes a set of instructions for certain operations described in this disclosure, is stored in a memory 408, which is also connected to the bus 406. The memory 408 can also store a user interface module to generate visual presentations.

Some embodiments of this disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations described herein. The media and computer code may be those specially designed and constructed for the purposes of this disclosure, or they may be of the kind available to those having skill in the computer software arts. Examples of computer-readable storage media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLDs), and ROM and RAM devices. Examples of computer code include machine code, such as produced by a compiler, and files containing higher-level code that are executed by a computer using an interpreter or a compiler. For example, an embodiment of this disclosure may be implemented using Java, C++, or other object-oriented programming language and development tools. Additional examples of computer code include encrypted code and compressed code. Moreover, an embodiment of this disclosure may be downloaded as a computer program product, which may be transferred from a remote computing device (e.g., a server computer) to a requesting computing device (e.g., a client computer or a different server computer) via a transmission channel. Another embodiment of this disclosure may be implemented in hardwired circuitry in place of, or in combination with, machine-executable software instructions.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, concentrations, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

What is claimed is:

1. A method of determining regulatory activity of nucleotides in a genome, comprising:
designating a reference region of the genome;
designating a plurality of reporter tiles covering the reference region, wherein each reporter tile has a length L, the reporter tiles are offset from one another within the reference region with a step size s, and a ratio of s to L is 1:5 or less; and
generating a plurality of reporter constructs corresponding to the reporter tiles.

2. The method of claim 1, wherein the ratio of s to L is 1:10 or less.

3. The method of claim 1, wherein the ratio of s to L is 1:20 or less.

4. The method of claim 1, wherein s is 20 base pairs (bps) in length or less.

5. The method of claim 1, wherein s is 10 bps in length or less.

6. The method of claim 1, wherein each reporter construct includes a corresponding reporter tile of the reporter tiles and an identification nucleic acid tag paired with the corresponding reporter tile.

7. The method of claim 6, further comprising:
inserting the reporter constructs into expression vectors;
introducing the expression vectors into cells in which the tags of the reporter constructs are expressed; and
determining extents of the tags expressed in the cells, where an extent of each tag expressed is an indication of a regulatory activity of a corresponding reporter tile of the reporter tiles.

8. A composition comprising:
a plurality of reporter constructs, each of which includes:
an identification nucleic acid tag; and
a nucleic acid sequence corresponding to a reporter tile of a plurality of reporter tiles,
wherein the reporter tiles cover a reference region of a genome, each reporter tile has a length L, the reporter tiles are offset from one another within the reference region with a step size s, and a ratio of s to L is 1:5 or less.

9. The composition of claim 8, wherein the ratio of s to L is 1:10 or less.

10. The composition of claim 8, wherein the ratio of s to L is 1:20 or less.

11. A composition comprising:
a plurality of reporter constructs including
a first reporter construct which includes a first identification nucleic acid tag and a first nucleic acid sequence of length L; and
a second reporter construct which includes a second identification nucleic acid tag and a second nucleic acid sequence of length L,
wherein the first nucleic acid sequence and the second nucleic acid sequence have an overlapping number of sequential bps in common that is at least 3/4 of L and is less than L.

12. The composition of claim 11, wherein the overlapping number of sequential bps in common is at least 9/10 of L and is less than L.

13. The composition of claim 11, wherein the overlapping number of sequential bps in common is at least 19/20 of L and is less than L.

14. A method of analyzing regulatory activity of nucleotides in a genome, comprising:
providing measurements of activities of a plurality of reporter tiles covering a reference region of the genome, wherein each reporter tile has a length L, the reporter tiles are offset from one another within the reference region with a step size s, and each reporter tile covers L/s nucleic acid intervals of the step size s;
providing a probabilistic model relating the activities of the reporter tiles to activities of nucleic acid intervals of the step sizes within the reference region; and
using the probabilistic model, deriving an activity of each nucleic acid interval of the step size s within the reference region.

15. The method of claim 14, wherein the probabilistic model relates an activity of each reporter tile to activities of L/s nucleic acid intervals of the step size s covered by the reporter tile.

16. The method of claim 14, further comprising deriving an activity of each nucleotide within the reference region from the activities of the nucleic acid intervals of the step size s within the reference region.

17. The method of claim 16, wherein deriving the activity of each nucleotide includes performing piecewise linear interpolation.
